(19) United States
(12) Patent Application Publication
(10) Pub. No.: US 2010/0055116 A1
(43) Pub. Date: Mar. 4, 2010

(54) Methods and Compositions for Targeting c-Rel

(76) Inventors: Hsiou-chi Liou, New York, NY (US); Wenzhi Tian, Bayside, NY (US); Shuhua Cheng, New York, NY (US); Constance Y. Hsia, New York, NY (US); Alex Owyang, San Mateo, CA (US); Jason J. Hsu, New York, NY (US)

Correspondence Address: MEDLEN & CARROLL, LLP, 101 HOWARD STREET, SUITE 350, SAN FRANCISCO, CA, 94105, US

(21) Appl. No.: 12/226,128

(22) PCT Filed: Apr. 13, 2007
(86) PCT No.: PCT/US2007/009163

§ 371 Date: Oct. 14, 2009

Related U.S. Application Data
(60) Provisional application No. 60/791,877, filed on Apr. 13, 2006.

(51) Int. Cl. A61K 39/00; C12N 5/02; A61K 31/515; A61K 31/415; C07D 239/02; C07D 231/00; C07H 21/02

(52) U.S. Cl. 424/184.1; 435/375; 514/270; 514/404; 544/299; 548/366.1; 536/24.5

(57) ABSTRACT

The present invention relates to compositions and methods for targeting c-Rel. In particular, the present invention provides compositions and methods for treating cancers, inflammatory diseases, autoimmune diseases, and transplant rejection by inhibiting c-Rel activity and for regulating c-Rel for research and drug screening applications.

This application claims priority to provisional application Ser. No. 60/791,877, filed Apr. 13, 2006, which is herein incorporated by reference in its entirety.

This invention was funded in part by Grant Nos. awarded by CA68155 and CA90405. The government may have certain rights in the invention.

### FIELD OF THE INVENTION

The present invention relates to compositions and methods for targeting c-Rel. In particular, the present invention provides compositions and methods for treating cancer, autoimmune disease, allergy, inflammatory disease, transplant rejection, and bone loss, by inhibiting c-Rel activity, eliciting immune tolerance, and for regulating c-Rel for research and drug screening applications. The present invention is also directed to methods of screening for inhibitors of c-Rel activity as determined by assaying c-Rel-mediated biological activities.

### BACKGROUND OF THE INVENTION

Many human diseases including inflammation, autoimmune disease, and cancer are attributed to aberrant activation of transcription factors, which leads to dysregulated target gene expression and evidence of new biological activities as well as survival or proliferative advantages. In the transcription factor field, NF-kB has attracted central attention as being a transcription factor that is involved in a myriad of biological functions and pathological conditions including the regulation of innate and adaptive immune response to infection, inflammation, cell survival, and tumorigenesis.

NF-kB refers to the p50 (NF-kB1) and p65 (RelA) subunits that were initially isolated in the early 1990's by Dr. David Baltimore's group at the Whitehead Institute at MIT. Three other proteins, c-Rel, RelB, and p52 (NF-kB2) were found to share sequence homology at the Rel Homologous Domain (RHD). Hence, these 5 proteins are classified as the Rel transcription factor family. Despite the similarity, each Rel member is distinct with regard to tissue expression pattern, response to receptor signals, and target gene specificity. These differences are evident from the non-redundant phenotypes exhibited by individual Rel knockout mouse. Thus, therapeutics targeted to different Rel members have different biological effects and safety/toxicity profiles.

C-Rel is distinct from NF-kB (p50, p65). c-Rel is the cellular homolog of the v-Rel oncogene encoded by the avian REV-T retrovirus. Unlike the NF-kB p50 and p65 that are ubiquitously expressed in all of the cells of the body, c-Rel is exclusively expressed in cells of hematopoietic origin including T cells, B cells, macrophages, and dendritic cells. In addition, c-Rel and NF-kB regulate distinct sets of target genes in different cells. As a result, they have distinct biological functions. c-Rel is a key culprit in many of the inflammatory and autoimmune diseases.

Anti-inflammatory and immunosuppressive therapies for inflammation, autoimmune disease, transplantation have undergone revolutionary development in the past several decades. Early therapies for treating the symptoms of autoimmune/inflammatory disorders relied on glucocorticoids or corticosteroids, hormones from the adrenal medulla discovered in the 1950's. Glucocorticoids are extremely effective in dampening the signs and symptoms of inflammation and the resultant immunopathology in almost all inflammatory disorders, including rheumatoid arthritis, asthma, allergic dermatitis, inflammatory bowel disease, multiple sclerosis, transplant rejection, graft vs host (GvH) disease, and organ-specific autoimmune diseases such as thyroiditis and diabetes. Unfortunately, corticosteroids cause severe systemic side effects that impact almost all organ systems, and which preclude their chronic administration. Thus, the euphoria that corticosteroids might be “the cure” for chronic autoimmune and inflammatory diseases rapidly dissipated even before the 1960s.

Palliation of the symptoms of chronic inflammatory disorders such as rheumatoid arthritis are drugs classified as non-steroid anti-inflammatory drugs (NSAIDs). However, long-term use of many of these agents can cause gastrointestinal (GI) bleeding. In the 1990s, a new class of drugs known as selective inhibitors of Cox2 (Vioxx, Celebrex, Bextra) was developed to treat pain and inflammation but circumventing the NSAID's side effects on the GI tract. Both NSAID and Cox2 inhibitors only treat symptom and relief pain for autoimmune patients. These drugs, however, are unable to curb the progression of the disease process. In 2004, Vioxx was pulled off the market due to an increased incidence of heart attacks. A black-box label was also placed on Bextra. Subsequently, the sales of all Cox2 inhibitor drugs declined significantly as the cardiovascular risks appeared to be common in this class of drugs.

In the 1990's, a novel class of biologics that block tumor necrosis factor (TNF), an inflammatory cytokine, were developed. The three drugs in this class, Enbrel, Remicade and Humira, have had a major impact in slowing the joint damage caused by rheumatoid arthritis, and one of the drugs is also approved to treat psoriasis, Crohn's disease and ankylosing spondylitis. While these new biologics drugs have fewer side effects than steroids, they are very expensive and are associated with risk of infections and certain cancers. Moreover, 30-35% patients become refractory to anti-TNF therapy over time due to the production of neutralizing antibodies.

These facts make apparent the need for alternative safe and efficacious therapies that are also affordable for the treatment of inflammatory and autoimmune disease. As suggested by the success of the TNF-blocking class of drugs, a therapy that targets specific cellular proteins involved in the core disease mechanism of autoimmunity is most desirable since such a therapy will slow disease progression. Based on the fundamental function of c-Rel in immune cells, c-Rel blockade further finds use in the treatment of other pathological conditions including inflammation, autoimmune disease, bone loss, transplant rejection, lymphoma, and solid tumors.

Cancer remains an incurable disease. Most current cancer therapies such as chemotherapies have broad cellular targets and exhibit unbearable side-effects on the patients. The success of Gleevec in CML and other related cancers has proved the principle that targeted therapy can be achieved as long as the oncogenic target is identified. c-Rel was first characterized as a proto-oncogene in chicken. Subsequently, c-Rel gene amplification or constitutive activation has been documented in many human B cell leukemia, lymphoma, as well as tumors derived from solid tissues. Therefore, c-Rel is a novel therapeutic target for human cancers with over-reactive c-Rel or NF-kB activity.

### SUMMARY OF THE INVENTION

The present invention relates to compositions and methods for targeting c-Rel. In particular, the present invention provides compositions and methods for treating cancer, autoimmune disease, allergy, inflammatory disease, transplant rejection, and bone loss, by inhibiting c-Rel activity, eliciting immune tolerance, and for regulating c-Rel for research and drug screening applications. The present invention is also directed to methods of screening for inhibitors of c-Rel activity as determined by assaying c-Rel-mediated biological activities.

Accordingly, the invention provides methods and compositions for targeting c-Rel as a therapeutic target for inflammatory disorders and tumors, as well as for inducing immune tolerance for the treatment of autoimmune disease and transplant rejection.

For example, in some embodiments, the present invention provides c-Rel activity inhibitors (e.g., antisense, siRNA, aptamers, antibodies, peptides, peptidomimetics, small molecules, and natural compounds) for use in inhibiting c-Rel or c-Rel signaling partner activity. Such inhibitors find use in the treatment of cancer, autoimmune, transplant rejection, and inflammatory disease and in research and drug screening applications. For example, in some embodiments, the present invention provides a method of decreasing c-Rel activity, comprising contacting a cell expressing a c-Rel gene with a c-Rel activity inhibitor. In some embodiments, the c-Rel activity inhibitor is an antisense oligonucleotide, an siRNA (e.g., SEQ ID NO:6, 10, 13, 16 or 17-26), an aptamer, a peptide, a peptidomimetic, or an antibody. In other embodiments, the c-Rel activity inhibitor is a natural compound or small molecule. In some embodiments, the small molecule has a structure of

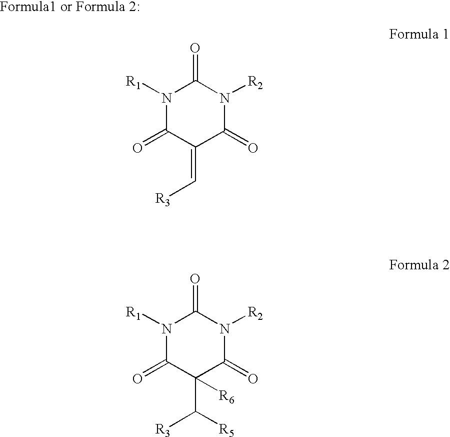

wherein R₁, R₂, R₅ and R₆ are independently selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl; R₃ is selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, CN, NO₂, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁; wherein R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl.

Preferred R₃ group is selected from aryl, substituted aryl, heterocyclic aromatic or non-aromatic, substituted heterocyclic aromatic or non-aromatic. For example,

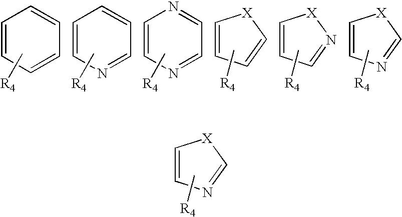

wherein X is selected from O, S, NH, NR₇. R₄ is independently selected hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, CN, NO₂, SO₂R₁1, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁, and wherein R₇, R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl.

In some embodiments, the small molecule has the structure:

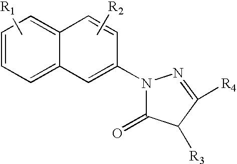

wherein R₁ and R₂ are independently selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, OH, OR₁₁, SH, SR₁₁, NO₂, CN, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁. R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. R₁₁ and R₁₂ can be connected to form a cycle which can be heterocyclic aromatic or non-aromatic, substituted heterocyclic aromatic, cycloakyl, substituted cycloalkyl. R₃ and R₄ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. R₃ and R₄ can be connected to form a cycle which can be heterocyclic aromatic or non-aromatic, substituted heterocyclic aromatic, cycloakyl, substituted cycloalkyl.

In some embodiments the small molecule comprises the formula:

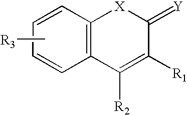

wherein X and Y are independently selected from NH, NR₄, O and S. R₁, R₂ and R₄ are independently selected from hydrogen, aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocylic aromaric or non-aromatic, substituted heterocyclic aromatic or non-aromatic, cycloalkyl, substituted cycloalkyl. R₁ and R₂ can be connected to form a cycle which can be heterocyclic, substituted heterocyclic, cycloakyl, substituted cycloalkyl. R₃ is selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, COR₁₁, OH, OR₁₁, SH, SR₁₁, NO₂, CN, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁. R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. R₁₁ and R₁₂ can be connected to form a cycle which can be heterocyclic aromatic or non-aromatic, substituted heterocyclic aromatic, cycloakyl, substituted cycloalkyl.

Exemplary compounds include, but are not limited to, 1,3-dimethyl-5-{3-[2-(4 nitrophenoxy)ethoxy]benzylidene}-2,4,6(1H,3H,5H)-pyrimidinetrione, 1,3-dimethyl-5-[3-(2-phenoxyethoxy)benzylidene]-2,4,6(1H,3H,5H)-pyrimidinetrione, 1,3-[(tetrahydro-1-methyl-2,4,6-trioxo-5(2H)-pyrimidinylidene)methyl)phenoxy]-Acetic acid, 4-[(tetrahydro-1-methyl-2,4,6-trioxo-5(2H)-pyrimidinylidene)methyl]-Benzoic acid, 5-[3-bromo-4-(dimethylamino)benzylidene]-2,4,6(1H,3H,5H)-pyrimidinetrione, 5-[[4-(dimethylamino)-3-nitrophenyl]methylene]-2,4,6(1H,3H,5H)-Pyrimidinetrione, 5-[(5-chloro-2-methoxyphenyl)methylene]-1,3-dimethyl-2,4,6(1H,3H,5H)-Pyrimidinetrione, 5-[[2-[(2-chlorophenyl)methoxy]phenyl]methylene]-1,3-dimethyl-2,4,6(1H,3H,5H)-Pyrimidinetrione, 5-[[3-[(2-chlorophenyl)methoxy]phenyl]methylene]-1,3-dimethyl-2,4,6(1H,3H,5H)-Pyrimidinetrione, 5-[[2-[(4-chlorophenyl)methoxy]phenyl]methylene]-1,3-dimethyl-2,4,6(1H,3H,5H) Pyrimidinetrione, 2,4,6(1H,3H,5H)-Pyrimidinetrione, 5,5′-(1,4-phenylenedimethylidyne)bis-(9CI) or Barbituric acid, 5,5′-(p-phenylenedimethylidyne)di-(8CI); 5,5′-p Xylenediylidenebis(barbituric acid), Benzonitrile, 2-[2-methoxy-4-[(tetrahydro-1,3-dimethyl-2,4,6-trioxo-5(2H)-pyrimidinylidene)methyl]phenoxy]-5-nitro-(9CI) 2,4,6(1H,3H,5H)-Pyrimidinetrione, or 5-[[3-chloro-5-methoxy-4-[2-(4-methylphenoxy)ethoxy]phenyl]methylene]-(9CI)). In other embodiments, the small molecule is 1H-Pyrazole-1-butanoic acid, 3-(4-bromophenyl)-5-(1,2-dihydro-7-methyl-2-oxo-3-quinolinyl)-4,5-dihydro-g-oxo-(9CI) 1,5-Naphthalenedisulfonic acid, 3-(4,5-dihydro-3-methyl-5-oxo-1H-pyrazol-1-yl)-1,3-Naphthalenedisulfonic acid, 7-(3-methyl-5-oxo-2-pyrazolin-1-yl)-(8CI) Butanedioic acid, or [5-[(4-hydroxy-3-methoxyphenyl)methylene]-4-oxo-2-thioxo-3-thiazolidinyl]. In still further embodiments, the small molecule is 4-Hydroxy-2-oxo-1,2-dihydroquinoline-3-carboxylic acid N-(4-hydroxyphenyl)amide or 7-(diethylamino)-3-[5-(2,5-dimethoxyanilino)-1,3,4-thiadiazol-2-yl]-2H-chromen-2-one. In some embodiments, the cell is a human cell, a cancer cell, B-lymphocyte, a T-lymphocyte, an antigen presenting cell, or an inflamed cell. In some embodiments, the cell is in an organism (e.g., a human or a non-human mammal). In some embodiments, the human exhibits symptoms of cancer (e.g., those described in Table 1). In other embodiments, the human exhibits symptoms of an allergy, inflammatory or autoimmune disease (e.g., those described in Table 1). In still further embodiments, the human has undergone an organ transplant (e.g., those described in Table 1).

In some embodiments, inhibition of c-Rel results in a phenotype selected from the group including, but not limited to, cell growth arrest, apoptosis, immune suppression, and immune tolerance induction. In some embodiments, inhibiting c-Rel activity comprises reducing binding of c-Rel to c-Rel recognition sites on c-Rel target genes. In other embodiments, inhibiting c-Rel activity comprises interrupting the interaction of c-Rel with a c-Rel transcription co-activator, transcription mediator, or other transcription factors. In yet other embodiments, inhibiting c-Rel activity comprises preventing c-Rel modification by upstream signaling molecules, co-factors, or enzymes. In still further embodiments, inhibiting c-Rel activity comprises altering c-Rel structural conformation to an inactive state.

The present invention further comprises a method of inhibiting the expression of a c-Rel target gene (e.g., a soluble factor, a cytokine, a cell cycle regulator, or a cell survival protein), comprising contacting a eukaryotic cell expressing a c-Rel gene with a c-Rel activity inhibitor.

The present invention additionally provides a method, comprising contacting a eukaryotic cell exhibiting abnormal signaling effects, wherein the abnormal signaling effects result in altered c-Rel mediated activity (e.g., increased or decreased activity) with a c-Rel activity inhibitor under conditions such that the abnormal signaling effect is diminished.

The present invention also provides a method for modifying extracellular signaling influences on a eukaryotic cell, wherein the extracellular signaling induces c-Rel-mediated biological activity, comprising contacting the cell with a c-Rel activity inhibitor under conditions such that the signaling effect is decreased.

In certain embodiments, the present invention provides a method of treating a disease caused by excessive c-Rel activity, comprising administering a c-Rel activity inhibitor to a subject exhibiting symptoms of the disease. In some embodiments, the disease is an inflammatory disease (e.g., acute respiratory distress, sepsis, hepatitis, colitis, inflammatory bowel disease, ischemia-reperfusion injury, or atherosclerosis), an autoimmune disease (e.g., lymphoproliferative disease, systemic lupus erythematosis, rheumatoid arthritis, multiple sclerosis, or ankylosing spondylitis), bone loss (e.g., bone loss is derived from arthritis, bone loss derived from inflammation, or bone loss derived from autoimmune disease), organ transplant rejection (e.g., graft vs. host disease or bone marrow transplant rejection), immune therapy (e.g., induction of immune tolerance), and cancer (e.g., B cell lymphoma, Burkitt's lymphoma, chronic lymphocytic leukemia, multiple myeloma, lymphoma with Pten mutation, leukemia with Pten mutation, Cowden's syndrome, tumors with Pten mutation, prostate cancer, breast cancer, metastatic tumor hepatocellular carcinoma, colon cancer, or gastrointestinal cancer).

The present invention additionally provides a method of treating a disease caused by aberrant expression of a c-Rel target gene, comprising administering a c-Rel activity inhibitor to a subject exhibiting symptoms of the disease (e.g., those diseases disclosed herein).

The present invention further provides a kit comprising a c-Rel activity inhibitor in a pharmaceutically acceptable carrier. In some embodiments, the c-Rel activity inhibitor is an antisense oligonucleotide, an siRNA (e.g., SEQ ID NO:6, 10, 13, 16 or 17-26), an aptamer, a peptide, a peptidomimetic, or an antibody. In other embodiments, the c-Rel activity inhibitor is a natural compound or a small molecule. In some embodiments, the small molecule has a structure as described above.

The present invention also provides a composition comprising a small molecule, wherein the small molecule has a structure as described above.

The present invention also provides a composition comprising a siRNA for regulating c-Rel activity. In some embodiments, the siRNA has the nucleic acid sequence of SEQ ID NO:6, 10, 13, 16 or 17-26 or functional equivalents.

In still further embodiments, the present invention provides a method of screening compounds, comprising contacting a human c-Rel homodimer with a binding partner (e.g., CD28 response element (CD28RE) in the promoter region of IL-2 gene); and measuring the level of fluorescence polarization in the presence of a test compound relative to the levels in the absence of a test compound. In some embodiments, the assay is a high throughput assay. In some embodiments, test compounds are further screened using an electrophoretic mobility shift assay.

In additional embodiments, the present invention provides a method of decreasing c-Rel activity, comprising providing an inhibitor of c-Rel signaling pathway component and downstream target genes. In some embodiments, the signaling pathway component is a receptor molecule upstream of c-Rel activation (e.g., TCR, BCR, CD40, TNF receptor family, NOD1, NOD2, and Toll-like receptors) that is triggered by its cognate ligands. In other embodiments, the signaling pathway component is a signaling molecule upstream of c-Rel activation (e.g., Lyn, Fyn, Lck, PI3-kinase, Pten, Akt, Vav, BSAP, SLP-76, LAT, Itk, Btk, ZAP-70, PKC-beta, PKC-theta, PKC-zeta, Bcl-10, MALT1, CARMA1, IKKα, IKKβ, IKKγ, NIK, TRAFs, TAK1, TBK1, RIP, MyD88, TIRAP, TRAM, and TRIF). In another embodiment, c-Rel downstream target genes include, but are not limited to, cytokines (e.g. IL-2, IL-3, GM-CSF, IFN-γ, IFN-α, TNF, IL-6, IL-8, IL-10, IL-13, IL-15, IL-12, IL-23, IL-17, IL-27, EBI3, MIP1α, Rantes, VEGF), cytokine receptors (e.g. IL-2Rα, IFN-α receptor, OCILRP1, NKRP1f, amphiregulin, angiopoietin-like, N-EGF2, FGF1, Bmp-1), costimulatory molecules (e.g. CD80, CD86, CD40, CD44, CCR7, CXCR4, ICAM-1, VCAM-1, MM-9), cell cycle proteins (e.g. cyclin D1, cyclin D2, cyclin D3, cyclin E, E2Fs, ifi202), cell survival proteins (e.g. Bcl-X, Bfl-1, Mcl-1. c-IAPs, c-FLIP, A20), signaling molecules (e.g. IKK-I, MKK1, GBP-1, Pim1, Rap1, R-Rad, Map13K, PLA-gamma), and transcription factors (e.g. c-myc, JunB, IRF1, IRF4, Stat5a, B-Atf, Tbx-2, Cited2, Pvit1, Cri1, Siah2, Hox-8). In some embodiments, two or more of the pathways components are targeted.

Other embodiments of the invention are described in the description and examples below.

### DESCRIPTION OF THE FIGURES

FIG. 1 shows an overview of the fluorescent polarization screen assay used in some embodiments of the present invention.

FIG. 2 shows the characterization of specific dose-dependent interactions between purified human c-Rel protein and the recognized kB site CD28RE. Left panel, sample electrophoretic mobility shift assays of c-Rel homodimer. Right panel, logarithmic plot of DNA binding data from EMSA with c-Rel homodimer.

FIG. 3 shows the development and validation of an assay to identify small molecule inhibitors of the transcription factor c-Rel. 3a, various concentrations of c-Rel were added to 10 nM of FITC-labeled CD28 RE probe, and fluorescence polarization was measured. 3b, Fluorescence polarization of different concentrations of the unlabeled CD28 RE probe and the control oligonucleotide Oct1. 3c, 200 nM of endogenous inhibitor IkBa of c-Rel was added into the reaction containing 10 nM of the probe, 100 nM c-Rel and other components same as 3b. 3d, scatter distribution of FP data from an example 384-well plate.

FIG. 4 shows further identification of small molecule inhibitors of c-Rel by EMSA.

FIG. 5 shows that lymphoma cells derived from Pten-mutant mice have a high proliferative response to BCR signal. (A) Histology of Pten(+/−) lymph node derived lymphomas. (B) Pten(+/−) lymphoma cells were cultured in medium in the presence or absence of anti-IgM for up to 72 hours. Cell cycle and apoptosis were analyzed by propidium iodide (PI) staining followed by flow cytometry.

FIG. 6 shows that Pten-mutant splenocytes and B cells exhibit spontaneous proliferation and accelerated cell cycle progression in response to BCR and CD40 signals. (A) Splenocytes or (B) B cells isolated from the Pten+/− and the wild type mice were cultured with either medium alone (upper panel) or anti-IgM (lower panel). (C) The percentages of B cells over each cycle after stimulation for 72 hr is shown inside each histogram, from the left to the right, 0, 1st, 2nd, 3rd, 4+5th, cycle.

FIG. 7 shows that Pten-mutant B cells exhibit sustained NF-kB activation kinetics and express high levels of c-Rel target gene EBI3. (A) EMSA of nuclear extracts derived from Pten-mutant and control B cells stimulated with anti-IgM for up to 6 hours. (B) EMSA of nuclear extracts derived from Pten(hypo-allele) B cells. (C) EBI3 is constitutively upregulated in Pten-mutant B cells.

FIG. 8 shows that pharmacological NF-kB inhibitors effectively block cell cycle progression and induce mitochondrial apoptotic process of Pten-mutant B cells. CFSE assay (A) or propidium iodide staining (B) was used to assess cell division. (C) Bay 11 and Velcade treatment led to mitochondrial depolarization of both Pten-mutant and control B cells.

FIG. 9 shows that C-Rel deletion leads to apoptosis and cell cycle arrest of Pten-mutant B cells. B cells derived from mice with either c-Rel or Pten deletion or both were analyzed for apoptosis and cell cycle progression by using PI staining (A) or 3tt-thymidine incorporation assay (B).

FIG. 10 shows in vitro silencing of c-Rel. (a) GFP levels in NIH3T3 cells treated with 100 μl of c-Rel siRNA expressing retrovirus or control virus (virus titer: 5×106/ml). (b) Western blot with c-Rel specific polygonal antibody.

FIG. 11 shows that silencing c-Rel results in diminished cell survival and cell cycle progression in B cell lymphoma Wehi-231 cells. (a) percentage of GFP⁺ cells. (b) Cell survival and cell cycle progression analyzed by PI staining. (c) Data in (b) summarized as a line chart.

FIG. 12 shows that in vitro silencing of c-Rel leads to impaired cell survival and cell cycle progression in primary B cells in response to antigenic and mitogenic signals. (a) flow cytometry showing infection efficiency. (b) Cell survival and cell cycle progression was analyzed by PI staining. (c) Cells harvested from the same culture in (a) were stained with anti-Ki-67 using intracellular staining methods and analyzed by flow cytometry.

FIG. 13 shows that in vivo silencing of c-Rel leads to impaired cell survival and cell cycle progression in primary B cells in response to antigenic and mitogenic signals. (a) ³H thymidine incorporation of primary B cells isolated from the chimeric mouse spleen and stimulated for 48 hr respectively with anti-IgM (10.0 μg/ml), anti-CD40 (10.0 μg/ml), and LPS (10.0 μg/ml). (b) B cells stimulated with anti-CD40 in (a) were further analyzed respectively by PI staining for cell survival and cell cycle progression, and by Ki-67 staining for cell proliferation.

FIG. 14 shows that in vivo silencing of c-Rel results in an impaired T cell-mediated immune response to antigen specific signals.

FIG. 15 shows exemplary small molecules of the present invention.

FIG. 16 shows exemplary Class I, II and III compounds of the present invention. (a) Class II compound inhibits c-Rel. (b) Class I compound inhibition of DNA probe binding with c-Rel as Class I compound concentration increases.

FIG. 17 shows the synthesis of exemplary compounds of the present invention.

FIG. 18 shows the synthesis of exemplary compounds of the present invention.

FIG. 19 shows the synthesis of derivatives of exemplary compounds of the present invention.

FIG. 20 shows a synthetic scheme to incorporate radiochemical or stable isotope labels into compounds of the present invention.

FIG. 21 shows modifications in the carboxylate moieties of Class II compounds of the present invention.

FIG. 22 shows neutral sulfone or sulfonamide group modification of the compounds of the present invention.

FIG. 23 demonstrates cellular potency of c-Rel inhibitor compounds on IL-2 expression in T cells. (a) Intracellular IL-2 staining on T cells stimulated with anti-CD3 and anti-CD28. The data show that C04, a Class I compound, reduces IL-2 expression in T cells. (b, c) Dose-dependent inhibition of IL-2 production by T cells in the presence of some compounds of the present invention. (d, e). Varying doses of C04 and C01 were used to measure IC50 of the c-Rel inhibitor compounds in inhibiting IL-2 production by CD4+ or CD8+ T cells.

FIG. 24 is a model that illustrates that activation of Rel/NF-kB is required for immunogenic response. This model also proposes that overactivation of c-Rel/NF-kB activity in tolerogenic cells can lead to immune tolerance breakdown and onset of autoimmune disease.

FIG. 25 shows that immature B cells, which undergo immune tolerance or deletion, have specific suppression of c-Rel and NF-kB activation.

FIG. 26 shows specific impaired activation of the PI3K signaling pathway in immature B cells that undergo immune tolerance and deletion.

FIG. 27 shows that Pten mutation (or deletion) can restore survival and proliferative response to tolerogenic lymphocytes, thus forming a basis for immune tolerance breakdown and autoimmunity.

FIG. 28 show that Akt, a downstream effector of the PI3K/Pten pathway, can protect tolerogenic lymphocytes from apoptosis or deletion.

FIG. 29 lists selected c-Rel target genes identified by comparing c-Rel wild type and c-Rel knockout lymphocytes stimulated with BCR (B cell antigen receptor) using DNA microarray technology.

FIG. 30 lists selected c-Rel target genes identified by comparing c-Rel wild type and c-Rel knockout lymphocytes stimulated with CD40 using DNA microarray technology.

### DEFINITIONS

To facilitate an understanding of the present invention, a number of terms and phrases are defined below:

As used herein the term “c-Rel activity” refers to any biochemical and biological activity of c-Rel including, but not limited to expression, binding to a binding partner (e.g. other NF-kB members including, but not limited to, p50, p52, p65, and RelB), binding to DNA with a specific Rel-responsive-sequence, phosphorylation, acetylation, and other post-translational modification, nuclear translocation, transcription activity, regulation of target gene transcription and expression, interaction with co-activators, co-repressors, or mediators in the nucleus, interaction with RNA Polymerase II, interaction with other transcription factors (e.g., STATs, IRFs, c-jun, c-fos, Foxp3, and NF-ATs) on the same promoters of the c-Rel target genes, and signaling activity. C-Rel activity includes activity mediated through interactions with other proteins or nucleic acids or responding to upstream receptor signaling pathways. For example, in some embodiments, c-Rel biological activity refers to c-Rel activation by signaling pathway emanated by cognate ligand binding to its corresponding receptors (e.g., TCR, BCR, CD40, TNF receptor family, NOD1, NOD2, and Toll-like receptors). In other embodiments, c-Rel biological activity is modulated by a signaling component of c-Rel (e.g., Lyn, Fyn, Lck, PI3-kinase, Pten, Akt, Vav, BSAP, SLP-76, LAT, Itk, Btk, ZAP-70, PKC-beta, PKC-theta, PKC-zeta, Bcl-10, MALT1, CARMA1, IKKα, IKKβ, IKKγ, NIK, TRAFs, TAK1, TBK1, RIP, MyD88, TIRAP, TRAM, and TRIF). In another embodiments, c-Rel activity refers to modulation of c-Rel downstream target genes including, but not limited to, cytokines (e.g. IL-2, IL-3, GM-CSF, IFN-γ, IFN-α, TNF, IL-6, IL-8, IL-10, IL-13, IL-15, IL-12, IL-23, IL-27, EBI3, MIP1α, Rantes, VEGF), cytokine receptors (e.g. IL-2Rα, IFN-α receptor, OCILRP1, NKRP1f, amphiregulin, angiopoietin-like, N-EGF2, FGF1, Bmp-1), costimulatory molecules (e.g. CD80, CD86, CD40, CD44, CCR7, CXCR4, ICAM-1, VCAM-1, MMP-9), cell cycle proteins (e.g. cyclin D1, cyclin D2, cyclin D3, cyclin E, E2Fs, ifi202), cell survival proteins (e.g. Bcl-X, Bfl-1, Mcl-1, c-IAPs, c-FLIP, A20), signaling molecules (e.g. IKK-1, MKK1, GBP-1, Pim1, Rap1, R-Rad, Map13K, PLA-ganuna), and transcription factors (e.g. c-myc, JunB, IRF1, IRF4, Stat5a, B-Atf, Tbx-2, Cited2, Pvit1, Cri1, Siah2, Hox-8).

As used herein, the term “inhibitor of c-Rel activity” refers to any molecule (e.g., siRNA, anti sense nucleic acid, aptamer, antibody, peptide, peptidomimetic, natural compound, or small molecule” that decreases any activity of c-Rel (e.g., including, but not limited to, the activities described herein), via directly contacting c-Rel protein, contacting c-Rel mRNA, causing conformational changes of c-Rel, decreasing c-Rel protein levels, or interfering with c-Rel interactions with signaling partners (e.g., those described herein), and affecting the expression of c-Rel target genes (e.g. those described herein). Inhibitors also include molecules that indirectly regulate c-Rel biological activity by intercepting upstream signaling molecules (e.g. PI3K, Pten, IKKs).

As used herein, the term “abnormal signaling” refers to alterations in signaling pathways or components of the signaling pathways that lead to abnormal cellular response (e.g., in growth, survival, apoptosis, differentiation, metabolism, effector function, or gene expression pattern). In some embodiments, abnormal signaling results in altered c-Rel activity. In some embodiments, abnormal signaling is the result of altered activity of a c-Rel target gene or c-Rel upstream signaling molecule (e.g., those described herein).

As used herein, the term “extracellular signaling influences” refers to the effect that extracellular signaling molecules (e.g., pharmaceutical agents, ligands to a receptor, cytokines, chemokines, soluble factors, adhesion molecules, or other signaling molecules) have on a cell (e.g., a eukaryotic cell). In some embodiments, extracellular signaling induces c-Rel activity, alters c-Rel activation kinetics, or alters c-Rel target gene expression pattern.

As used herein, the term “substituted aliphatic” refers to an alkane possessing less than 10 carbons where at least one of the aliphatic hydrogen atoms has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “alkyl” denotes branched or unbranched hydrocarbon chains, preferably having about 1 to about 8 carbons, such as, methyl, ethyl, n-propyl, iso-propyl, n-butyl, sec-butyl, iso-butyl, tert-butyl, 2-methylpentyl pentyl, hexyl, isohexyl, heptyl, 4,4-dimethyl pentyl, octyl, 2,2,4-trimethylpentyl and the like.

As used herein, the term “substituted alkyl” includes an alkyl group optionally substituted with one or more functional groups which are attached commonly to such chains, such as, hydroxyl, bromo, fluoro, chloro, iodo, mercapto or thio, cyano, alkylthio, heterocyclyl, aryl, heteroaryl, carboxyl, carbalkoyl, alkyl, alkenyl, nitro, amino, alkoxyl, amido, and the like to form alkyl groups such as trifluoro methyl, 3-hydroxyhexyl, 2-carboxypropyl, 2-fluoroethyl, carboxymethyl, cyanobutyl and the like.

As used herein, the term “cycloalkyl” as employed herein alone or as part of another group includes saturated or partially unsaturated (containing 1 or more double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 10 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl and cyclododecyl, cyclohexenyl.

As used herein, the term “substituted cycloalkyl” includes a cycloalkyl group optionally substituted with 1 or more substituents such as halogen, alkyl, alkoxy, hydroxy, aryl, aryloxy, arylalkyl, cycloalkyl, alkylamido, alkanoylamino, oxo, acyl, arylcarbonylamino, amino, nitro, cyano, thiol and/or alkylthio and/or any of the substituents included in the definition of “substituted alkyl.”

As used herein, the term “alkenyl” by itself or as part of another group refers to straight or branched chain radicals of 2 to 20 carbons, preferably 2 to 12 carbons, and more preferably 2 to 8 carbons in the normal chain, which include one or more double bonds in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, and the like. “Substituted alkenyl” includes an alkenyl group optionally substituted with one or more substituents, such as the substituents included above in the definition of “substituted alkyl” and “substituted cycloalkyl.”

As used herein, the term “alkynyl” by itself or as part of another group refers to straight or branched chain radicals of 2 to 20 carbons, preferably 2 to 12 carbons and more preferably 2 to 8 carbons in the normal chain, which include one or more triple bonds in the normal chain, such as 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl, 3-undecynyl, 4-dodecynyl and the like. “Substituted alkynyl” includes an alkynyl group optionally substituted with one or more substituents, such as the substituents included above in the definition of “substituted alkyl” and “substituted cycloalkyl.”

As used herein, the terms-“arylalkyl”, “arylalkenyl” and “arylalkynyl” as used alone or as part of another group refer to alkyl, alkenyl and alkynyl groups as described above having an aryl substituent. Representative examples of arylalkyl include, but are not limited to, benzyl, 2-phenylethyl, 3-phenylpropyl, phenethyl, benzhydryl and naphthylmethyl and the like. “Substituted arylalkyl” includes arylalkyl groups wherein the aryl portion is optionally substituted with one or more substituents, such as the substituents included above in the definition of “substituted alkyl” and “substituted cycloalkyl.”

As used herein, the term “halogen” or “halo” as used alone or as part of another group refers to chlorine, bromine, fluorine, and iodine.

As used herein, the terms “halogenated alkyl”, “halogenated alkenyl” and “alkynyl” alone or as part of another group refers to “alkyl”, “alkenyl” and “alkynyl” which are substituted by one or more atoms selected from fluorine, chlorine, bromine, fluorine, and iodine.

As used herein, the term “aryl” or “Ar” alone or as part of another group refers to monocyclic and polycyclic aromatic groups containing 6 to 10 carbons in the ring portion (such as phenyl or naphthyl including 1-naphthyl and 2-naphthyl) and may optionally include one to three additional rings fused to a carbocyclic ring or a heterocyclic ring (such as aryl, cycloalkyl, heteroaryl or cycloheteroalkyl rings).

As used herein, the term “substituted aryl” includes an aryl group optionally substituted with one or more functional groups, such as halo, haloalkyl, alkyl, haloalkyl, alkoxy, haloalkoxy, alkenyl, trifluoromethyl, trifluoromethoxy, alkynyl, cycloalkyl-alkyl, cycloheteroalkyl, cycloheteroalkylalkyl, aryl, heteroaryl, arylalkyl, aryloxy, aryloxyalkyl, arylalkoxy, alkoxycarbonyl, arylcarbonyl, arylalkenyl, aminocarbonylaryl, arylthio, arylsulfinyl, arylazo, heteroarylalkyl, heteroarylalkenyl, heteroarylheteroaryl, heteroaryloxy, hydroxy, nitro, cyano, amino, substituted amino wherein the amino includes 1 or 2 substituents (which are alkyl, aryl or any of the other aryl compounds mentioned in the definitions), thiol, alkylthio, arylthio, heteroarylthio, arylthioalkyl, alkoxyarylthio, alkylcarbonyl, arylcarbonyl, alkylaminocarbonyl, arylaminocarbonyl, alkoxycarbonyl, aminocarbonyl, alkylcarbonyloxy, arylcarbonyloxy, alkylcarbonylamino, arylcarbonylamino, arylsulfinyl, arylsulfinylalkyl, arylsulfonylamino or arylsulfonaminocarbonyl and/or any of the alkyl substituents set out herein.

As used herein, the term “heterocyclic” or “heterocycle” represents an unsubstituted or substituted stable 5- to 10-membered monocyclic ring system which may be saturated or unsaturated, and which consists of carbon atoms and from one to four heteroatoms selected from N, O or S, and wherein the nitrogen and sulfur heteroatoms may optionally be oxidized, and the nitrogen heteroatom may optionally be quaternized. The heterocyclic ring may be attached at any heteroatom or carbon atom which results in the creation of a stable structure. Examples of such heterocyclic groups include, but are not limited to, piperidinyl, piperazinyl, oxopiperazinyl, oxopiperidinyl, oxopyrrolidinyl, oxoazepinyl, azepinyl, pyrrolyl, pyrrolidinyl, furanyl, thienyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isooxazolyl, isoxazolidinyl, morpholinyl, thiazolyl, thiazolidinyl, isothiazolyl, thiadiazolyl, tetrahydropyranyl, thiamorpholinyl, thiamorpholinyl sulfoxide, thiamorpholinyl sulfone, and oxadiazolyl.

As used herein, the term “heterocyclic aromatic” alone or as part of another group refers to a 5- or 7-membered aromatic ring which includes 1, 2, 3 or 4 hetero atoms such as nitrogen, oxygen or sulfur and such rings fused to an aryl, cycloalkyl, heteroaryl or heterocycloalkyl ring (e.g. benzothiophenyl, indolyl), and includes possible N-oxides. As used herein, the term “substituted heteroaryl” includes a heteroaryl group optionally substituted with 1 to 4 substituents, such as the substituents included above in the definition of “substituted alkyl” and “substituted cycloalkyl.”

As used herein, the term “cycloaliphatic” refers to a cycloalkane possessing less than 8 carbons or a fused ring system consisting of no more than three fused cycloaliphatic rings. Examples of such include, but are not limited to, decalin and the like.

As used herein, the term “substituted cycloaliphatic” refers to a cycloalkane possessing less than 10 carbons or a fused ring system consisting of no more than three fused rings, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl, bicyclo-heptanes, octanes, and nonanes (e.g., norbornyl) and the like.

As used herein, the term “substituted heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “linker” refers to a chain containing up to and including eight contiguous atoms connecting two different structural moieties where such atoms are, for example, carbon, nitrogen, oxygen, or sulfur. Ethylene glycol is one non-limiting example.

As used herein, the term “lower-alkyl-substituted-halogen” refers to any alkyl chain containing up to and including eight carbon atoms where one of the aliphatic hydrogen atoms is replaced by a halogen. Examples of such include, but are not limited to, chlorethyl and the like.

As used herein, the term “acetylamino” shall mean any primary or secondary amino that is acetylated. Examples of such include, but are not limited to, acetamide and the like.

The term “derivative” of a compound, as used herein, refers to a chemically modified compound wherein the chemical modification takes place either at a functional group of the compound or on the aromatic ring.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IgE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, peptides or immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “non-human transgenic animal lacking a functional c-Rel gene” refers to a non-human animal (preferable a mammal, more preferably a mouse) whose endogenous c-Rel gene has been inactivated (e.g., as the result of a “c-Rel knockout” or a “c-Rel knock-in”).

As used herein, the terms “c-Rel knockout” refers to an animal (e.g., a mouse) lacking a functional c-Rel gene. In some embodiments, the entire c-Rel gene is deleted. In other embodiments, the gene is inactivated via other means (e.g., deletion of essential portions or inversions of some or all of the c-Rel gene). In other embodiments, the c-Rel gene is inactivated using antisense inhibition. c-Rel knockouts include conditional knockouts (e.g., selective inhibition of gene activity). c-Rel knockout mice may be made using any suitable method including, but not limited to, those described herein. c-Rel genes can also be inactivated via the construction of a “c-Rel knock-in” in which the gene is inactivated by the insertion of exogenous DNA into a region of the gene required for function.

As used herein, the term “c-Rel mimetic” refers to a small molecule compound that mimics the binding of a c-Rel to a ligand.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., mRNA, rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such as temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 μl NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer or inflammatory disease). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present invention.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the terms “anticancer agent,” “conventional anticancer agent,” or “cancer therapeutic drug” refer to any therapeutic agents (e.g., chemotherapeutic compounds and/or molecular therapeutic compounds), radiation therapies, or surgical interventions, used in the treatment of cancer (e.g., in mammals).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative, antineoplastic, anti-inflammatory, immunosuppressive, and immunomodulatory compounds as well as other biologically therapeutic compounds.

### DETAILED DESCRIPTION OF THE INVENTION

The present invention relates to compositions and methods for targeting c-Rel. In particular, the present invention provides compositions and methods for treating cancer, autoimmune disease, allergy, inflammatory disease, transplant rejection, and bone loss, by inhibiting c-Rel activity, eliciting immune tolerance, and for regulating c-Rel for research and drug screening applications. The present invention is also directed to methods of screening for inhibitors of c-Rel activity as determined by assaying c-Rel-mediated biological activities.

The primary function of an immune system is to defend a host against infection or invasion by foreign subjects including bacteria, viruses, parasites, allergens, and allo-tissues. Upon encountering the foreign antigens or infected by pathogenic agents, the host is capable of mounting an inflammatory immune response to destroy and contain the foreign agents. Immune cells involved in the inflammatory response include innate immune cells such as macrophages, dendritic cells, neutrophils, and granulocytes, as well as adaptive immune cells such as T-lymphocytes and B-lymphocytes. The collaboration and interaction among immune cells leads to proliferation and differentiation of the antigen-specific lymphocytes as well as the production of inflammatory cytokines and mediators, resulting in destruction of infected cells and containment of foreign agents. Organ transplant rejection is in essence an immune response to foreign tissues. While inflammation is a self-defense mechanism, an uncontrolled inflammatory response can lead to acute distress syndrome or chronic exacerbation of the symptom. In those incidences, an anti-inflammatory or immunosuppressive medicine is often administered to control the unabated immune response.

While the immune system is capable of recognizing wide varieties of foreign antigens, it also evolves a mechanism to “tolerate” self-tissues or self-antigens; a mechanism termed “immune tolerance”. Immune tolerance to self-antigen is achieved because self-antigen-reactive lymphocytes are either deleted during lymphocyte development or rendered “unresponsive or anergic” by the self-antigen. Recently, T-regulatory cells (T-reg) have also been suggested to contribute to the suppression of self-reactive lymphocytes. Autoimmune disease is ensued when host immune tolerance mechanism is breached and the self-reactive lymphocytes become activated, thus attacking self-tissues.

In 1960, the mechanism of the immunosuppressive effects of steroids was found to reside in their capacity to block the proliferation of activated lymphocytes. As well, in the 60 s, in addition to their anti-inflammatory/immunosuppressive effects, steroids were found to be effective anticancer agents, particularly for lymphomas and leukemias. However, despite their impressive capabilities to dampen inflammation and to kill malignant lymphocytes, the molecular mechanism(s) responsible for the suppressive effects went unrecognized until 1980, when Dr. Kendall A. Smith and his team demonstrated that steroids block the production of interleukin-2 (IL2) by T lymphocytes (T cells). Since IL2 is the T cell growth factor responsible for the proliferation and survival of T cells during an immune response, this finding explained a great deal as to why steroids are such effective immunosuppressive agents. Subsequently, it was demonstrated that cyclosporin-A and tacrolimus (FK506), new immunosuppressive drugs found to be very effective in blocking rejection of organ transplants, also work by blocking IL2 production, as well as the production of other inflammatory cytokines.

In 1990 the mechanism whereby steroids are capable of producing such widespread immunosuppressive effects was ultimately traced to the inhibition of a family of molecules termed Nuclear Factor kappa B (NF-kB), which regulate the transcriptional activation of many genes, including the genes encoding IL2 as well as many other similar cytokine molecules such as Tumor Necrosis Factor (TNF), which mediate inflammatory responses. Due to the homology of NF-kB to v-Rel and c-Rel, this transcription factor family is now called the Rel family. There are 5 molecular members of Rel family, among which the NF-kB (p50, p65) complexes are distributed in all of the cells of the body, whereas c-Rel is predominantly expressed in immune cells.

NF-kB refers to the p50 (NF-kB1) and p65 (RelA) subunits isolated in the early 1990's by Dr. David Baltimore's group at the Whitehead Institute at MIT. Three other proteins, c-Rel, RelB, and p52 (NF-kB2) were found to share sequence homology at the Rel Homologous Domain (RHD). Hence, these 5 proteins are classified as the Rel transcription factor family. Despite the similarity, each Rel member is distinct with regard to tissue expression pattern, response to receptor signals, and target gene specificity. These differences are evident from the non-redundant phenotypes exhibited by individual Rel knockout mouse. Thus, therapeutics targeted to different Rel members are likely to have different biological effects and safety/toxicity profile.

C-Rel is distinct from NF-KB (p50, p65). NF-kB is described, for example, in U.S. Pat. No. 6,410,516, which is herein incorporated by reference. First, c-Rel was isolated as the cellular homolog of the v-Rel oncogene encoded by the avian REV-T retrovirus. C-Rel was thus recognized as a proto-oncogene 6 years prior to the discovery of NF-kB in 1990. Second, unlike NF-KB p50 and p65, which are ubiquitously expressed in all of the cells of the body, c-Rel is exclusively expressed in cells of hematopoietic origin including T cells, B cells, macrophages, and dendritic cells. Third, c-Rel and NF-kB regulate distinct sets of target genes in different cells. As a result, they have distinct biological functions. Fourth, many of the inflammatory responses initially ascribed to NF-kB were in fact largely attributed to c-Rel (as c-Rel is the predominant complex in immune cells as compared to NF-kB). This is supported by studies in c-Rel knockout mice. In the mid 1990's, two lines of c-Rel knockout mice were independently generated. C-Rel knockout mice develop normally but their immune functions are defective, such as the capacity to produce IL2 and other cytokines, consistent with its role only in activated immune cells. Blocking c-Rel in mice ameliorates asthma, experimental autoimmune, diabetes, and transplant rejection in animal models. C-Rel blockade in animal models also prevented onset of collagen-induced arthritis. These studies demonstrate that c-Rel is the key player in inflammatory and autoimmune disease processes owing to its predominate roles in immune cells (e.g. lymphocytes, dendritic cells, macrophages) and that the presence of NF-kB in the immune cells fail to compensate the loss of c-Rel function.

C-Rel is important for both lymphoid and myeloid cell functions. C-Rel is required for lymphocyte response to antigenic and costimulatory signals (e.g. BCR, TCR, CD28, CD40, CD30, Blys, TNF receptors) that are the core of adaptive immunity. Specifically, c-Rel regulates immune cell proliferation and survival, as well as cytokine production. C-Rel regulates the expression of cell cycle proteins (e.g. Cyclin E) and cell survival proteins (e.g. Bcl-X, Bfl-1, Mcl-1). C-Rel is also required for antigen presenting cell (e.g. dendritic cells) maturation and costimulatory functions via the regulation of costimulatory molecules and cytokines. c-Rel is a versatile cytokine regulator that controls the expression of T cell cytokines (IL-2, IFN-γ, TNF, IL-17), B cell cytokines (IL-6, IL-10, IL-15), and dendritic cell cytokines (IL-12, IL-23, IL-27).

The aforementioned roles of c-Rel in many aspects of immune cell functions indicates that c-Rel is a key culprit in many of the inflammatory and autoimmune diseases and that blocking c-Rel protects or prevents the onset of those diseases. Indeed, c-Rel blockade has been shown to be beneficial in preventing the onset of several disease models in animals (e.g., asthma, experimental autoimmune encephalomyelites, collagen induced arthritis, diabetes, pancreatic islet transplantation, and heart transplantation). Based on the fundamental function of c-Rel in immune cells, it is contemplated that c-Rel blockade is also beneficial for treating the following pathological conditions (see Table 1), some of which are exemplified in the present invention.
A. Acute and chronic inflammation: Inflammation in the lung and respiratory system induced by allergens or viral and bacterial infection is caused by the infiltration of immune cells to the lung that produce inflammatory cytokines or allergic mediators (e.g. IgE). In the situation of acute respiratory distress syndrome (ARDS) caused by viral (e.g. influenza virus, bird flu virus H5N1, SARS virus) and bacterial infection can be deadly, as the “cytokine storm” produced by infiltrating immune cells can lead to lung edema and impair gas exchange of the lungs. Sepsis is yet another acute response manifested by systemic release of inflammatory cytokines and mediators due to severe bacterial invasion into the bloodstream. At present, there is no effective therapy for ARDS and sepsis. Hepatitis, colitis, inflammatory bowel diseases, and atherosclerosis are other examples of unresolved chronic inflammation in specific tissues. In each of these cases, NF-kB has been shown to play a pathological role, and therapeutic agents (commercial or experimental) that are effective in treating these disorders have been shown to block NF-kB activation. Many studies have shown that Rel family member activation is activated during ischemia and that Rel family activation is responsible for ischemia reperfusion injury of multiple organs including brain, heart, and kidney. Most studies only focus on the role of NF-kB (p50, p65) in the aforementioned pathological conditions, without addressing the role of c-Rel. The present invention provides c-Rel as an important inflammatory mediator for these organ-specific inflammatory diseases as well as reperfusion tissue injury. Taken together, the present invention provides methods and compositions for inhibiting c-Rel as a therapy for ARDS, respiratory inflammatory disorders, sepsis, organ-specific inflammation, and ischemic injury.
B. Autoimmune diseases: Autoimmune diseases arise from the host immune system attacking its own tissues. There are at least 80 autoimmune diseases afflicting various tissues such as joints (rheumatoid arthritis), central nervous system (multiple sclerosis), intestine (Crohn's disease), and skin (psoriasis). It is estimated that autoimmune diseases affect 5 to 8 percent of the American population, or up to 23.5 million people. Previous studies on c-Rel knockout mice have demonstrated that blocking c-Rel activity protects the animals from developing autoimmune encephalomyelitis, type I diabetes, and collagen-induced arthritis. The present invention provides methods and compositions for blocking c-Rel in the treatment of the following autoimmune diseases. Recent success of anti-TNF therapy in treating patients with rheumatoid arthritis and ankylosing spondylitis suggest that inflammatory cytokines play important pathological roles in these diseases. Since c-Rel is involved in the expression of many of the inflammatory cytokines including TNF and IL-6, the present invention provides methods and compositions for blocking c-Rel as a therapeutic in these diseases. Autoimmune diseases arise from the breakdown of immune tolerance to self-tissues or self-antigens. If the antigen is widely expressed (e.g. nuclear DNA), then the disease is systemic. By contrast, if the self-antigen is only expressed in a particular tissue (e.g. insulin), then the disease is tissue-specific (e.g. pancreatic cells in the case of diabetes). Recent advances in immunology have identified many genes whose expression or alteration is associated with the onset of tissue-specific or systemic autoimmune diseases. Most of these genes have functions in modulating antigen receptor (TCR/BCR) activation threshold, in which c-Rel is a key effector of the antigen-receptor signaling pathway. Therefore, the present invention provides methods and compositions for specific inhibition of c-Rel activity in autoreactive immune cells as a therapeutic for tissue-specific and systemic autoimmune diseases, including, but not limited to, rheumatoid arthritis, multiple sclerosis, diabetes, Crohn's disease, Grave's diseases, Hashimoto's thyroiditis, myasthenia gravis, Psoriasis, systemic lupus erythematosus (SLE), lymphoproliferative disease (ALPS), and Sjogren's syndrome.
C. Organ transplantation: It has been well documented that host T cells are primarily responsible for the rejection of allografts provided by HLA-mismatched donors. Such activation of host T cells is mediated via TCR-interaction with allo-MHC molecules on the graft. Since c-Rel is responsible for TCR-mediated T cell proliferation and effector function, the present invention provides methods and compositions for blocking c-Rel in host immune cells as an immunosuppressive agent and treatment and prevention of allograft rejection. c-Rel inhibitors find use as immunosuppressive agents in the transplantation a number of tissues, including, but not limited to, bone marrow, major organs (heart, lung, kidney, liver), as well as soft tissues (skin, cartilage, bone). In other embodiments, c-Rel suppression is used in the prevention of graft vs. host disease.
D. Immune tolerance induction: Current immunosuppressive therapies such as Cycloxporin, FK506, and glucocorticoids can cause adverse effects, which impose serious problems for patients with chronic disease. In addition, general immunosuppression also makes the host more susceptible to spontaneous infection. Therefore, the current trend in the field of immunology is to develop immunotherapeutic strategies with the goal of inducing tissue- or antigen-specific immune tolerance for the treatment of auto immune diseases as well as for preventing allograft rejection. Immune tolerance to a specific antigen (or tissue) can be achieved through three major mechanisms: deletion, anergy, and T-regulatory cells. Experiments conducted during the course of development of the present invention demonstrated that c-Rel inhibition is associated with these three immune tolerance mechanisms. First, it has been shown that anergic T cells and anergic B cells, which are unresponsive to TCR/BCR stimulation, have specific blocks in the c-Rel and NF-kB pathway. Second, studies on immature B cells, which undergo deletion, have a specific block in their c-Rel/NF-kB and PI3K activation pathway. Conversely, activation of the PI3K-Rel/NF-kB pathway in tolerant cells can lead to immune tolerance breakdown and the onset of autoimmune diseases (Example 8). Third, recent studies have shown that suppression of the NF-kB/Rel and NFAT by FoxP3 is involved in the suppression of effector T cell function T-regulatory cells. Finally, there is ample evidence to support that blocking NF-kB/Rel activity in dendritic cells can prevent maturation of dendritic cells and that such immature dendritic cells induce T cell tolerance or T-regulatory cell differentiation. Thus, it is contemplated that the c-Rel/PI3K pathway is the signaling integration point for determining immune tolerance vs. autoimmunity. More specifically, it is contemplated that sustained activation of this pathway leads to autoimmune diseases, whereas suppression of this pathway induces immune tolerance.
E. Bone loss: C-Rel and NF-kB have been shown to be involved in bone loss and the osteoporosis process. Several studies have shown that IKK-beta leads to the activation of c-Rel, RelB, and RelA (p65) in osteoclasts, which leads to osteoclast survival and inflammation-induced bone loss. Indeed, knockout out p50/p52 of the NF-kB members led to osteopetrosis and inhibiting IKK activity blocks osteoclastogenesis and prevents arthritic bone destruction. Thus, in some embodiments, the present invention provides methods and compositions for inhibiting conventional NF-kB or c-Rel for the prevention of arthritic or inflammation-mediated bone destruction.

Taken together, c-Rel is a therapeutic target for autoimmune diseases, inflammation, organ transplantation, and bone loss. Accordingly, in some embodiments, the present invention provides c-Rel inhibitors that reduce the production of multiple inflammatory cytokines, the expression of costimulatory molecules, and the expression of cell survival and cell cycle regulators in lymphocytes. As a result, c-Rel inhibitors dampen the activation of major types of immune cells: T-lymphocytes, B-lymphocytes, dendritic cells, and macrophages at the core of the immunopathological conditions. The present invention provides c-Rel inhibitors as adjuvant agents for inducing immune tolerance or the development of T-regulatory as novel therapies for autoimmune diseases and transplant rejection.

Another important feature relevant to drug safety/toxicity profile is that the lack of c-Rel activity in c-Rel knockout mice does not have a serious impact on systemic development, metabolism, or reproduction, nor does it cause cardiac fibrosis as seen in Cox2 knockout mice. This unique safety property is desirable, as it suggests that c-Rel inhibitors will not cause adverse effects as opposed to Cox2 inhibitors. In addition, targeting c-Rel avoids the systemic toxicities of corticosteroids and Cycloxporin/FK506, as well as the cardiac toxicity of Cox2 inhibitors.

C-Rel was initially identified as a proto-oncogene. Its viral counterpart v-Rel oncogene primarily transforms and immortalizes immature and mature T and B lymphoid, myeloid and dendritic cells from spleen and bone marrow and induces aggressive fatal lymphoma in infected young birds. The oncogenic potential of v-Rel was further demonstrated by experiments that demonstrated that transgenic mice expressing v-Rel under the control of T-cell tropic promoter developed aggressive T-cell leukemia/lymphoma in mice.

c-Rel is also associated with many cancers in human, due to its ability to prevent apoptosis (by inducing anti-apoptotic proteins) and to induce proliferation (via induction of cell cycle regulators). The fact that c-Rel is predominantly expressed in hematopoietic cells makes it one of the most prevalent oncoprotein in many B cell leukemias and lymphomas (Table 1). For example, the human c-Rel locus is amplified in a significant proportion of diffuse large cell lymphoma (23%), primary mediastinal B-cell lymphoma, follicular B-cell lymphoma, and Hodgkin's lymphoma. C-Rel gene rearrangement or over-expression is also detected in diffuse large cell lymphoma, follicular lymphoma, and non-small cell lung carcinoma. Additionally, constitutive or hyper-activate NF-kB/Rel has been detected in human B cell tumors including chronic lymphocytic leukemia (CLL). Freshly isolated unstimulated CLL B cells contain high levels of nuclear NF-kB/Rel activity consisting of c-Rel, p50, and p65. NF-kB/Rel activity can be further induced by CD40, which correlates with prolonged survival of the CLL cells in vitro. Other examples of B cell tumors that exhibit aberrant c-Rel activation include multiple myeloma, Burkitt's lymphoma, and mantle cell lymphoma. In some embodiments, the present invention provides evidence that blocking c-Rel reduces proliferation and survival of B cell lymphoma and tumor cells with Pten deletion (Example 3, Example 4). The present invention is not limited to a particular mechanism. Indeed, an understanding of the mechanism is not necessary to practice the present invention. Nonetheless, it is contemplated that based on the observation that tumor B cells have acquired survival advantage in vivo, it is contemplated that constitutive c-Rel and/or NF-kB activity contributes to tumor cell survival. NF-kB/Rel transcription factors are known to regulate multiple anti-apoptotic molecules including Bcl-x, Bcl-2, Mcl-2, LAP, and FLIPs. These observations make NF-kB/Rel family attractive therapeutic targets for treating B cell tumors, T cell leukemia, as well as Hodgkin and non-Hodgkin's diseases.

In addition to lymphoid tumors, aberrant constitutive Rel/NF-kB activity has been found in many non-hematopoietic tumors and solid carcinoma, including breast cancer, prostate cancer, melanoma, colon cancer, ovarian cancer, and non-small cell lung cancer. For instance, transgenic mice with human c-Rel gene under the control of the mouse mammary tumor virus (MMTV) long terminal repeat promoter develop mammary tumors with an average latency of 19.9 months. A high percentage of human breast tumors and tumor-derived cell lines have increased levels of constitutive nuclear NF-kB activity consisting of c-Rel, p50, Rel-B, and Bcl-3, and inhibiting NF-kB activity lead to cytotoxicity of the breast tumor cell lines. In some cases, activation of the Rel/NF-kB activity is coincident with malignant progression into metastasis or resistance to chemotherapy. Such progression may be attributed to the role of Rel/NF-kB in inducing genes involved in survival, proliferation, migration, and angiogenesis.

Experiments conducted during the course of development of the present invention demonstrated that suppression of c-Rel activity attenuates hyper-proliferation and lymphoma resulting from mutations in the Pten gene (Example 3). The Pten gene is a tumor suppressor frequently mutated in a variety of solid tumors including metastatic prostate cancers, endometrial cancers, metastatic melanoma, and glioblastomas. Mutations of the Pten gene have also been documented in over 80% of individuals with Cowden's disease (CD). Pten mutations were also found in a variety of B cell lymphomas. Specifically, a link between the hyperproliferative nature of the B cells and lymphoma cells derived from Pten-mutant mice and sustained activation and expression of NF-kB/Rel and its downstream target genes was identified. In addition, blocking NF-kB activity, by pharmacological inhibitors or c-Rel knockout mice, led to effective suppression of proliferation and induction of apoptosis of Pten-mutant cells.

Epidemiological studies have shown that ˜15% of human deaths from cancer are associated with chronic viral or bacterial infections, suggesting a link between infection, inflammation, and cancer. For example, HCV infection is an important risk factor for hepatocellular carcinoma (HCC). A bacterium, Helicobactor pylori, is one of the main contributors to gastric cancer, the second most common cancer worldwide. It has been hypothesized that activation of Rel/NF-kB by the classical IKK-dependent pathway is a crucial mediator of inflammation-induced tumor growth and progression. In fact, the hypothesis has been supported by two animal models: inflammation-associated liver cancer (a model for hepatoma) and inflammation-associated colon cancer (a model for colitis-associated cancer). These models suggest that Rel/NF-kB may promote tumor progression through inducing the expression of genes that encode secreted cytokines, growth factors, survival proteins, proteases, as well as factors for chemotaxis, migration, and angiogenesis. Accordingly, in some embodiments, the present invention provides methods and compositions for targeting c-Rel in inflammation-associated cancers. In some embodiments, the present invention provides c-Rel activity inhibitors for the treatment of infection or chemical-induced malignancies including, but not limited to, HCC, colon cancer, gastrointestinal cancer, lung cancer, pancreatic cancer, bladder cancer, and esophagus cancer.

Although there have been several reports on developing drugs or compounds that inhibit NF-kB and IKK signaling pathways, most of these studies focused on the NF-KB p50/p65 components. WO 2005/046619 (herein incorporated by reference in its entirety) describes compositions and methods for modulating c-Rel-dependent cytokine production.

Experiments conducted during the course of development of the present invention identified a sequence in the c-Rel-encoding mRNA sequence that can specifically silence c-Rel protein expression. Silencing of c-Rel lead to apoptosis and suppression of cell cycle progression of the B cell lymphoma cell line Wehi-231 (Examples 4, 5). Silencing c-Rel in primary B cells renders the cells more susceptible to apoptosis induction and decreases proliferative responses to CD40 signaling. In vivo silencing of c-Rel resulted in dramatic impairment in T cell mediated immune response to antigenic stimulation.

Further experiments conducted during the course of development of the present invention identified a series of small molecules that inhibit c-Rel activity (See e.g., Example 1 and Example 6 below).

Accordingly, in some embodiments, the present invention provides methods of treating cancer, inflammatory, autoimmune disease, transplant rejection, and bone loss by inhibiting c-Rel signaling. For example, in some embodiments, the present invention provides methods of inhibiting c-Rel activity to suppress antigen-mediated immune responses, elicit antigen-mediated immune tolerance (e.g., self-antigens, self-tissue, allergens, allo-antigens, allo-tissues, pathogenic bacterial or viral epitopes), and suppress chronic or acute inflammation (e.g., ARDS, sepsis, asthma, colitis, see Table 1). In other embodiments, inhibition of c-Rel activity is used to suppress autoreactive hyper-reactive lymphocyte function (e.g., autoimmune diseases such as lupus, rheumatoid arthritis, etc, see Table 1). In yet other embodiments, inhibition of c-Rel activity is used as an immunosuppressive therapy for transplantation. In still further embodiments, inhibition of c-Rel activity is used to induce growth arrest and induce apoptosis of tumors with constitutive c-Rel activity or PI3K/Pten abnormality (e.g., B cell lymphoma, CLL, multiple myeloma, non-Hodgkin, prostate cancer, Cowden diseases, and breast cancer, see Table 1).

Exemplary methods of inhibiting c-Rel signaling pathway and its downstream target genes are discussed in greater detail below and include, but are not limited to, modulating PI3K/Pten pathways that affect c-Rel and target gene expression including immune cell receptors (e.g. key BCR/TCR, TNF receptor family, NOD1, NOD2, and Toll-like receptors) and signaling components that are known to regulate c-Rel, (e.g. Lyn, Fyn, Lck, PI3-kinase, Pten, Akt, Vav, BSAP, SLP-76, LAT, Itk, Btk, ZAP-70, PKC-beta, PKC-theta, PKC-zeta, Bcl-10, MALT1, CARMA1, IKKα, IKKγ, NIK, TRAFs, TAK1, TBK1, RIP, MyD88, TIRAP, TRAM, and TRIF, etc), inhibiting c-Rel and its downstream target genes including, but not limited to, cytokines (e.g. IL-2, IL-3, GM-CSF, IFN-γ, IFN-α, TNF, IL-6, IL-8, IL-10, IL-13, IL-15, IL-12, IL-17, IL-23, IL-27, EBI3, MIP1α, Rantes, VEGF), cytokine receptors (e.g. IL-2Rα, IFN-α receptor, OCILRP1, NKRP1f, amphiregulin, angiopoietin-like, N-EGF2, FGF1, Bmp-1), costimulatory molecules (e.g. CD80, CD86, CD40, CD44, CCR7, CXCR4, ICAM-1, VCAM-1, MMP-9), cell cycle proteins (e.g. cyclin D1, cyclin D2, cyclin D3, cyclin E, E2Fs, ifi202), cell survival proteins (e.g. Bcl-X, Bfl-1, Mcl-1. c-LAPs, c-FLIP, A20), signaling molecules (e.g. IKK-1, MKK1, GBP-1, Pim1, Rap1, R-Rad, Map13K, PLA-gamma), and transcription factors (e.g. c-myc, JunB, IRF1, IRF4, Stat5a, B-Atf, Tbx-2, Cited2, Pvit1, Cri1, Siah2, Hox-8).

Exemplary methods of inhibiting c-Rel activity include using antisense, siRNA, aptamers, antibodies, peptides, peptidomimetics, small molecules, and natural compounds.

### I. Disease Therapy and Analysis

In some embodiments, the present invention provides therapies for treating and/or analyzing cancer, inflammatory, organ transplant rejection and autoimmune disease. In some embodiments, methods inhibit c-Rel activity or biological functions (e.g., by inhibiting the interaction of c-Rel with binding partners). In other embodiments, methods inhibit function by modulating c-Rel upstream signaling regulators, c-Rel transcriptional activity, or c-Rel target gene expression. The present invention further provides drugs screening and research uses (e.g., to identify inhibitors of c-Rel activity). In some embodiments, additional inhibitors of c-Rel activity are identified using the drug screening applications disclosed herein.

The present invention is not limited to the treatment of a specific condition or disease. An exemplary, non-limiting list of specific cancer inflammatory, and autoimmune disease and conditions are provided in Table 1.

TABLE 1
Disease indications applicable for c-Rel specific therapies

| | Specific disease indications benefited from |
|---|---|
| | c-Rel inhibition |
| Inflammatory | Asthma and allergy |
| diseases | Inflammatory pulmonary syndrome |
| (acute and chronic) | Acute respiratory distress syndrome (ARDS) |
| | Neonatal chronic lung disease |
| | Chronic obstructive pulmonary disease (COPD) |
| | Gram positive sepsis |
| | Gram negative sepsis |
| | Culture negative sepsis |
| | Fungal sepsis |
| | Systemic inflammatory response syndrome |
| | Hepatitis |
| | Colitis |
| | Inflammatory bowel disease (IBD) |
| | Ischemia-reperfusion injury |
| | Atherosclerosis |
| | Glomerulonephritis |
| | Pemphigus vulgaris |
| | Idiopathic thrombocytopenic purpura |
| | Aphthous ulcer |
| | Irtis |
| | Conjunctivitis |
| | Keratoconjunctivitis |
| | Cutaneous lupus erythematosus |
| | Vaginitis |
| | Proctitis |
| | Drug eruptions |
| | Leprosy reversal reaction |
| | Erythema nodosum leprosum |
| | Polychronditis |
| | Endotoxemia |
| | Lyme arthritis |
| | Infectious meningitis |
| | Rubella arthritis |
| | Eczema |
| | Allergic contact dermatitis |
| | Hypersensitivity pnemonitis |
| | Encephalomyelitis |
| | Type IV hypersensitivity |
| | Drug sensitivity |
| | Cachexia |
| | Cystic fibrosis |
| | Neutropenic fever |
| | Urosepsis |
| | Meningococcemia |
| | Trauma/hemorage |
| | Burns |
| | Ionizing radiation exposure |
| | Acute pancreatitis |
| | Alcohol-induced hepatitis |
| | Chronic inflammatory pathologies |
| | Sickle cell anemia |
| | Nephrosis |
| | Atopic diseases |
| | Hypersensitivity reactions |
| | Allergic phinitis |
| | Hay fever |
| | Perennial rhinitis |
| | Endometriosis |
| | Urticaria |
| | Systemic |
| | Anaphalaxis |
| | Anti-receptor hypersensitivity reactions |
| | Immune tolerance therapy via co-administration |
| | of allergens |
| Autoimmune | Multiple Sclerosis (autoimmune encephalomyelitis) |
| diseases: | Type I diabetes |
| | Rheumatoid arthritis |
| | Ankylosing spondylitis |
| | Spondyloarthropathies |
| | Crohn's disease (inflammatory bowel disease) |
| | Grave's disease |
| | Hashimoto's thyroiditis |
| | Myasthenia gravis |
| | Psoriasis |
| | Systemic lupus erythematosus (SLE) |
| | Lymphoproliferative disease (ALPS) |
| | Sjogren's syndrome |
| | Autoimmune neuropathies |
| | Gullian-Barre syndrome |
| | Autoimmune uveitis |
| | Autoimmune hemolytic anemia |
| | Pernicious anemia |
| | Aplastic anemia |
| | Pure red cell anemia |
| | Autoimmune thrombocytopenia, |
| | Temporal arteritis |
| | Anti-phospholipid syndrome |
| | Vasculitides |
| | Wegener's granulomatosis |
| | Behcet's disease |
| | Dermatitis herpetiformis |
| | Pemphigus vulgaris |
| | Vitiligo |
| | Primary biliary cirrhosis |
| | Autoimmune hepatitis |
| | Autoimmune oophoritis and orchitis |
| | Autoimmune disease of the adrenal gland |
| | Scleroderma |
| | Polymyositis |
| | Dermatomyositis |
| | Autoimmune menagitis |
| | Autoimmune dermatitis |
| | Alopecia areata |
| | Autoimmune uveitis |
| | Allergic encephalomyelitis |
| | Interstitial lung fibrosis |
| | Seronegative arthropathies |
| | Sarcoidosis |
| | Orchitis/vasectomy reversal procedure |
| | Raynoud's disease |
| | Type B insulin-resistant diabetes |
| | Antibody-mediated cytotoxicity |
| | Type III hypersensitivity reactions |
| | POEMS syndrome |
| | Polyneuropathy |
| | Organomegaly |
| | Endocrinopathy |
| | Monoclonal gammopathy |
| | Skin changes syndrome |
| | Pemphigus |
| | Mixed connective tissue diseases |
| | Idiopathic Addison's disease |
| | Post-MI cardiotomy syndrome |
| | Wilson's disease |
| | Hemachromatosis |
| | Alpha-1-antitrypsin deficiency |
| | Osteoporosis |
| | Hypothalamic-pituitary-adrenal axis evaluation |
| | Familial hematophagocytic lymphohistiocytosis |
| | Pre eclampsia |
| | Okt3 therapy |
| | Anti-cd3 therapy |
| | Cytokine therapy |
| | Chemotherapy |
| | Radiation therapy |
| | Immune tolerance therapy via co-administration of |
| | self-antigens or self-tissues |
| Transplantation | Graft vs. host disease |
| rejection | Organ transplantation: |
| | kidney |
| | heart |
| | liver |
| | pancreas |
| | Islet cells |
| | lung |
| | bone marrow |
| | skin allograft |
| | cartilage |
| | bone graft |
| | small bowel |
| | fetal thymus implant |
| | parathyroid |
| | Xenograft rejection |
| | Allograft rejection |
| | Immune tolerance therapy via co-administration of |
| | allo-antigens or allo-tissues |
| Cancers: | Diffuse large cell lymphoma |
| | Follicular B cell lymphoma |
| | Chronic lymphocytic leukemia |
| | Multiple myeloma |
| | Burkitt's lymphoma |
| | Primary mediastinal B-cell lymphoma |
| | Hodgkin's lymphoma |
| | Non-Hodgkin's lymphoma |
| | Mantle cell lymphoma |
| | Mucosa-associated lymphoid tissue |
| | (MALT) lymphoma |
| | Childhood acute lymphoblastic leukemia |
| | Adult T-cell leukemia |
| | Acute lymphoblastic leukemia |
| | Chronic myelogenous leukemia |
| | Immunoblastic lymphoma |
| | Kaposi's sarcoma |
| | Cowden's syndrome (intestinal polyposis, |
| | thyroid cancer, breast cancer) |
| | Breast cancers |
| | Breast carcinoma |
| | Colon carcinoma |
| | Prostate carcinoma |
| | Ovarian carcinoma |
| | Endometrial cancers |
| | Non-small cell lung carcinoma |
| | Metastatic prostate cancers |
| | Metastatic melanoma |
| | Pancreatic carcinoma |
| | Thyroid carcinoma |
| | Bladder carcinoma |
| | Renal cell carcinoma |
| | Squamous cell carcinoma |
| | Nasopharyngeal carcinoma |
| | Glioblastoma |
| | Hepatocellular carcinoma (HCC) |
| | Colon cancer |
| | Gastrointestinal cancer |
| | Lung cancer |
| | Pancreatic cancer |
| | Bladder cancer |
| | Oesophagus cancer |

### A. Antisense and RNAi Therapies

In some embodiments, the present invention targets the expression of c-Rel or signaling partners. For example, in some embodiments, the present invention employs compositions comprising oligomeric antisense compounds, particularly oligonucleotides, for use in modulating the function of nucleic acid molecules encoding c-Rel or signaling partners thereof, ultimately modulating the amount of c-Rel expressed and impacting the expression of c-Rel downstream target genes. This is accomplished by providing antisense compounds (e.g., antisense oligonucleotides, siRNA, etc.) that specifically hybridize with one or more nucleic acids encoding c-Rel or a signaling partner thereof. The specific hybridization of an oligomeric compound with its target nucleic acid interferes with the normal function of the nucleic acid.

i. RNA Interference (RNAi)

In some embodiments, RNAi is utilized to inhibit c-Rel function. RNAi includes, but is not limited to, small interfering RNA (siRNA), small hairpin RNA (shRNA), and microRNA (miRNA). RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous to the target sequence in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Corners, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

Exemplary siRNA sequences for use in modulating the expression of c-Rel are described in Examples 4 and 5 below. The present invention is not limited to the described sequences. Additional sequences can be designed and tested (e.g., using the methods described herein).

ii. Antisense

In other embodiments, the present invention employs compositions comprising oligomeric antisense compounds, particularly oligonucleotides (e.g., those identified in the drug screening methods described below), for use in modulating the function of nucleic acid molecules encoding c-Rel or signaling partners thereof, ultimately modulating the amount of c-Rel or signaling partner expressed. This is accomplished by providing antisense compounds that specifically hybridize with one or more nucleic acids encoding c-Rel or a signaling partner thereof. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of c-Rel. In the context of the present invention, “modulation” means either an increase (stimulation) or a decrease (inhibition) in the expression of a gene. For example, expression may be inhibited to potentially treat cancer or inflammatory diseases.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a c-Rel protein. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone), —CH₂—O—N(CH₃)—CH₂—, —CH₂—N(CH₃)—N(CH₃)—CH₂—, and —O—N(CH₃]-CH₂—CH₂— [wherein the native phosphodiester backbone is represented as—O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

### B. Antibody Therapy

In other embodiments, the present invention provides antibodies that target c-Rel or c-Rel signal pathway components in cancer, inflammatory or autoimmune disease. In preferred embodiments, the antibodies used for therapy are humanized antibodies. Methods and compositions for generating antibodies are described below.

### C. Small Molecule Drugs

In still further embodiments, the present invention provides drugs (e.g., small molecule drugs) that treat cancer, inflammatory or autoimmune disease by inhibiting the biological activity of c-Rel or altering the biological activity of c-Rel pathway components. Exemplary small molecule drugs are described in Examples 1, 2, 6, and 7 below.

Experiments conducted during the course of development of the present invention identified three families of small molecule c-Rel inhibitors. Class I compounds contain one of the following generic formulas:

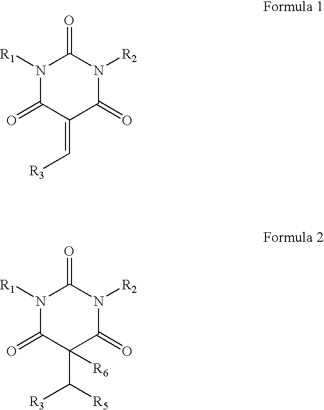

wherein R₁, R₂, R₅ and R₆ are independently selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. R₃ is selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, CN, NO₂, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁, wherein R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. Preferred R₃ group is selected from aryl, substituted aryl, heterocyclic aromatic or non-aromatic, substituted heterocyclic aromatic or non-aromatic. For example,

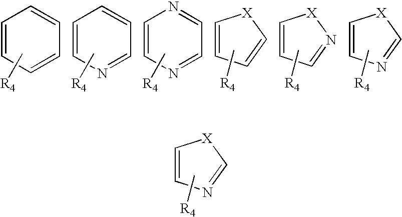

wherein X is selected from O, S, NH, NR₇. R₄ is independently selected hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, CN, NO₂, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁, and wherein R₇, R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cyclolakyl, substituted cycloalkyl.

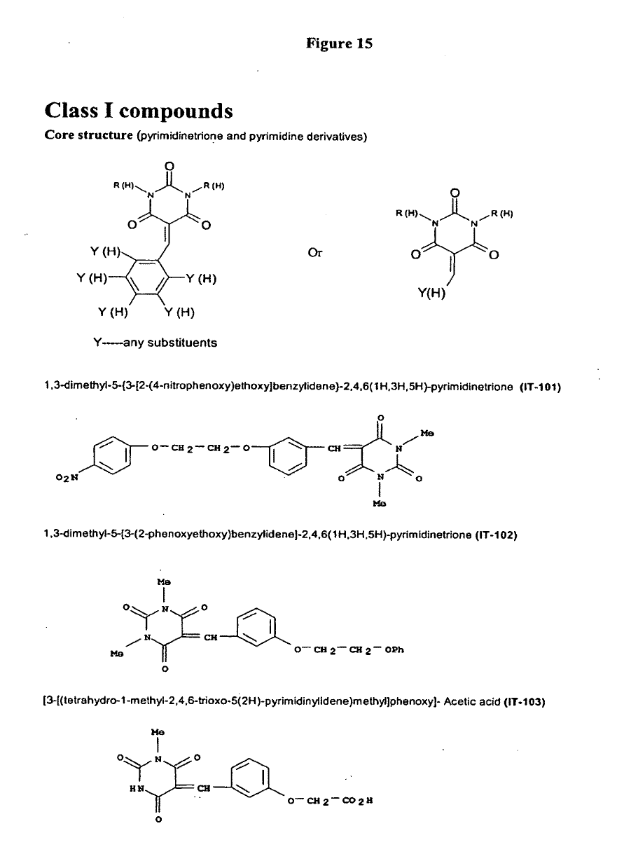

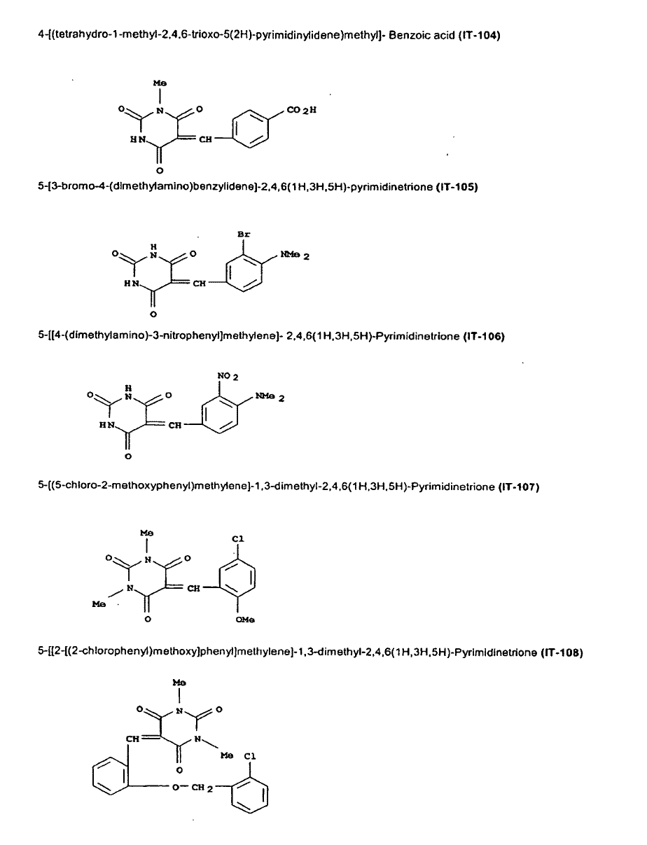

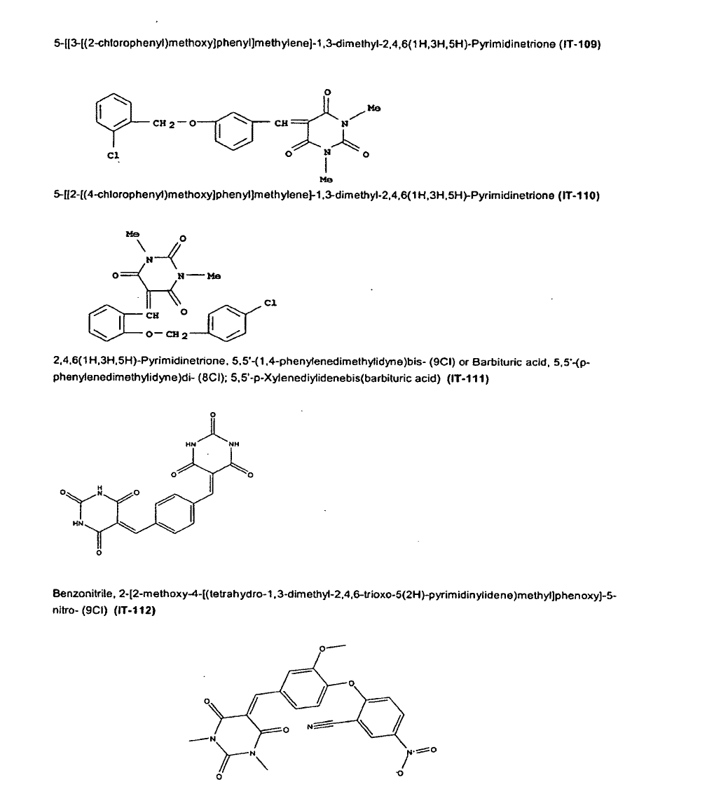

Class I compounds which contain the core structures as listed below are exemplified by IT101-113 (FIG. 15).

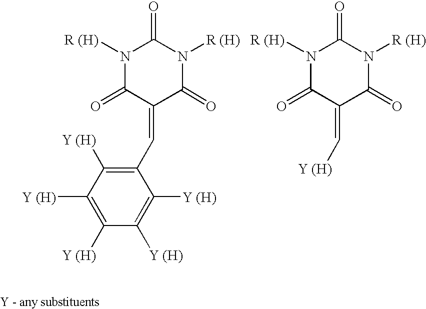

Class II compounds contain the following generic pyrazolone naphthalene scaffold

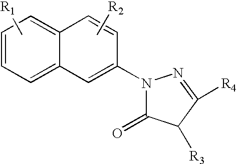

wherein R₁ and R₂ are independently selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, OH, OR₁₁, SH, SR₁₁, NO₂, CN, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁. R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. R₁₁ and R₁₂ can be connected to form a cyclic moiety, which can be heterocyclic aromatic or non-aromatic, substituted heterocyclic aromatic, cycloakyl, substituted cycloalkyl. R₃ and R₄ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. R₃ and R₄ can be connected to form a cycle which can be heterocyclic aromatic or non-aromatic, substituted heterocyclic aromatic, cycloakyl, substituted cycloalkyl.

Class II compounds are exemplified by IT202-203 (FIG. 15).

Class III compounds contain the following generic structure of formula 3:

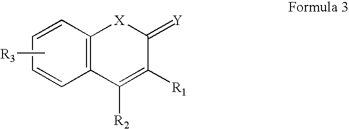

wherein X and Y are independently selected from NH, NR₄, O and S. R₁, R₂ and R₄ are independently selected from hydrogen, aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocylic aromaric or non-aromatic, substituted heterocyclic aromatic or non-aromatic, cycloalkyl, substituted cycloalkyl. R₁ and R₂ can be connected to form a cycle which can be heterocyclic, substituted heterocyclic, cycloakyl, substituted cycloalkyl. R₃ is selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, COR₁₁, OH, OR₁₁, SH, SR₁₁, NO₁₂, CN, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁. R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl. R₁₁ and R₁₂ can be connected to form a cycle which can be heterocyclic aromatic or non-aromatic, substituted heterocyclic aromatic, cycloakyl, substituted cycloalkyl.

Class III compounds are exemplified by IT201 and IT301-302 (FIG. 15).

The present invention is not limited to the compounds described herein. The present invention specifically contemplates chemical modifications and derivatives of the disclosed lead compounds.

In other embodiments, additional small molecule drugs are identified using the drug screening methods described below. In particularly preferred embodiments, the small molecule drugs of the present invention result in the inhibition or prevention of cancer, inflammatory or autoimmune disease.

### D. Aptamers

In other embodiments, aptamers are utilized as c-Rel inhibitors. Aptamers are unique RNA/DNA polymers that provide immense diversity in tertiary structures. This allows the selection of unique aptamers that specifically interact with the protein of interest. In some embodiments, aptamers are generated from 1 linear chain of DNA or RNA molecule. In other embodiments, branched DNA molecules in the form of Y-shape, X-shape, and T-shape are utilized. These branched DNA molecules increase the diversity of tertiary structures that cannot be achieved by conventional aptamers, resulting in a significantly expanded aptamer library and a much fast aptamer screening.

Aptamers are functional oligonucleotide sequences that have a specific affinity to targeting molecules (usually proteins), very much like an antibody-antigen interaction (Jayasena, 1999. Clin Chem 45:1628). A variety of aptamers have been generated by the Systematic Evolution of Ligands by Exponential Enrichment (SELEX) process (Uphoff et al., 1996. Curr Opin Struct Biol 6:281). In this process, a library of linear nucleic acid sequences that can be amplified is generated and is screened rapidly for sequences that have specific binding affinities with target molecules (e.g., c-Rel in this case). Candidates are then pooled and amplified via PCR. This selection and enrichment step reduces the complexity of the initial library dramatically and is repeated several times until positive sequences are identified. Aptamers have been successfully used as reagents for diagnosis (Gold, 1995. J Biol Chem 270:13581) and as inhibitors for therapeutics (Gold et al., 1995. Annu Rev Biochem 64:763).

In some embodiments, C-Rel protein with histidine-tag is purified from E. coli by using a Nickel column. Protein purity is evaluated by electrophoresis on denaturing SDS-PAGE. Protein activity is evaluated by electrophoretic mobility shift assay (EMSA).

In some embodiments, Y-DNA structures are designed. Each arm of Y-DNA is designed to have a long overhang (single stranded DNA or SSDNA) with random sequences, creating three potential aptamers that are physically in a branched formation. These branched aptamers have much more diversities and complexities. Similarly X-DNA and T-DNA can also be used to generate a large, diversified aptamer library. Once formed, Y-, X-, or T-DNA are quite stable.

In some embodiments, solid phase assays via a well-established 6×His-Ni binding is utilized to screen for DNA-aptamer complexes (a modified SELEX process). Briefly, a library of Y-DNA, X-DNA and T-DNA with random sequences on each arm is synthesized from a DNA synthesizer. 6×His-c-Rel is used to bind a Ni-coated 96-well. The aptamer library is incubated with attached c-Rel first and then washed extensively. Tentative binding aptamers are retained while unbound DNA is washed off. Amplification of Y-DNA-aptamers is carried out via PCR with two common primers and the entire process is repeated several times to enrich aptamers with high affinity to c-Rel. Candidates are further screened via a more stringent assay (e.g., the EMSA assay described in the experimental section below). Binding affinities are also determined. The sequences of final aptamers is determined via sequencing.

In additional embodiments, the combination of X, Y, and T is utilized to generate aptamers of different valencies (other than 3 as seen in Y-DNA-aptamers). This increases diversities even more. For example, Y-DNA can be ligated to Y-DNA itself. The branches are increased geometrically and increase valencies. By exploring different geometry and valencies, many sets of branched-DNA-aptamer libraries can be generated such that the number of free ends (which carry aptamer sequences) is tailored from 3 (Y-DNA), 4 (X-DNA) to almost any number desired. For example, a penta-valency (5 arms) can be achieved by simply ligating Y-DNA with X-DNA. Similarly, a hexa-valency (6 arms) can be made by simply joining two tetra-valent X-DNAs. Generally speaking, to create n free ends when n is an even number, all one needs to do is to ligate (n-2) Y-DNA. To create n arms when n is an odd number, it is more complicated but still very much achievable: one can always cut n-valency into two parts: an even number (m) and a small odd number (p) where n=m+p. An example of 11 valencies is shown here: 11=6+5. Both hexa-valency (6) and penta-valency (5) can be fabricated easily. By ligating hexa-penta DNA plus an extra Y, one gets a multivalent DNA with 11 branches. Using this strategy, any branched aptamer DNA can be assembled.

Aptamers are screened for their ability to prevent c-Rel protein from binding to the NF-kB site. Such candidates alter c-Rel tertiary confirmation or bind to the critical DNA-recognition phase of the c-Rel protein, thus interfering c-Rel interaction with the NF-kB site. Aptamers that exhibit inhibitory activity in EMSA assay are further tested with in vitro cellular assays (e.g., using an NF-kB-luciferase reporter assay). The NF-kB-luciferase reporter plasmid and the c-Rel aptamers are co-transfected into 3T3 fibroblast cell line at varying ratios. Forty eight hours later, cell lysates are prepared from these transfectants and assayed for luciferase activity using a luminometer. Desirable c-Rel aptamers inhibit the luciferase activity driven by the NF-kB promoter as an indication that they prevent c-Rel binding to the cognate NF-kB motif in the promoter region.

### E. Peptide and Pepdimominetic Therapies

In certain embodiments, peptide and peptidomimetic therapies are utilized to decrease c-Rel activity and/or expression. In some embodiments, therapies are peptides that interact with c-Rel or c-Rel pathway components to decrease or inhibit the biological activity of c-Rel.

The present invention further includes peptides modified to improve one or more properties useful in pharmaceutical compounds. For example, in some embodiments, peptides are modified to enhance their ability to enter intracellular space. Such modifications include, but are not limited to, the addition of charged groups, lipids, myristate groups (See e.g., U.S. Pat. No. 5,607,691; herein incorporated by reference), or cell-permeable peptides derived from HIV TAT peptide or antennapedia homeo-domain.

In other embodiments, the peptides of the present invention may be in the form of a liposome in which isolated peptide is combined, in addition to other pharmaceutically acceptable carriers, with amphipathic agents such as lipids which exist in aggregated form as micelles, insoluble monolayers, liquid crystals, or lamellar layers which in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

In other embodiments, peptidomimetics are utilized. A variety of designs for such mimetics are possible. For example, cyclic-containing peptides, in which the necessary conformation for binding is stabilized by nonpeptides, are specifically contemplated. U.S. Pat. No. 5,192,746, U.S. Pat. No. 5,169,862, U.S. Pat. No. 5,539,085, U.S. Pat. No. 5,576,423, U.S. Pat. No. 5,051,448, and U.S. Pat. No. 5,559,103, all hereby incorporated by reference, describe multiple methods for creating such compounds.

Synthesis of nonpeptide compounds that mimic peptide sequences is also known in the art. Eldred et al. (J. Med. Chem., 37:3882 [1994]) describe nonpeptide antagonists that mimic peptide sequences. Likewise, Ku et al. (J. Med. Chem., 38:9 [1995]) give further elucidation of the synthesis of a series of such compounds. Such nonpeptide compounds that mimic peptide inhibitors of the present invention are specifically contemplated by the present invention.

The present invention also contemplates synthetic mimicking compounds that are multimeric compounds that repeat the relevant peptide sequence. As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react.

For example, the α-amino group of the component containing the activated carboxyl group can be blocked with a tertbutyloxycarbonyl group. This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In one embodiment, the mimetics of the present invention are peptides having sequence homology to peptides with the desired activity. One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant (Pearson and Lipman, Proc. Natl. Acad. Sci. (USA), 85:2444-2448 (1988); Lipman and Pearson, Science, 227:1435 (1985)).

In some embodiments, peptides and peptidomimetics with the desired activity are identified using the drug screening assays described herein.

### F. Genetic And Transplantation Therapies

In yet other embodiments, the present invention contemplates the use of any genetic manipulation for use in modulating the expression of c-Rel. Examples of genetic manipulation include, but are not limited to, delivery of inhibitors of c-Rel (e.g., to cells, tissues, or subjects). Delivery of nucleic acid constructs to cells in vitro or in vivo may be conducted using any suitable method. A suitable method is one that introduces the nucleic acid construct into the cell such that the desired event occurs (e.g., expression of an siRNA construct alone or in combination with a therapeutic agent or targeting antigen). For example, cells may be transfected ex vivo to decrease c-Rel expression and the transfected cells may be transplanted to the site of a tumor or other disease.

Introduction of molecules carrying genetic information and/or therapeutic agent into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors, tissue associated with tumors, or inflamed tissues such as arthritic joints using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### G. Pharmaceutical Compositions

The present invention further provides pharmaceutical compositions (e.g., comprising the therapeutic compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the therapy is administered in maintenance doses, ranging from 0.01 μg to 100 g per kg of body weight, once or more daily, to once every 20 years.

H. Therapeutic Agents Combined or Co-administered with Anti-c-Rel Compounds

In some embodiments, the c-Rel targeting compounds of the present invention are coadministered with additional therapeutic agents. A wide range of therapeutic agents find use with the present invention. Any therapeutic agent that can be co-administered with compounds that target c-Rel or associated proteins.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, inhibit protein kinase activity, block receptors for growth factors, cytokines, activating ligands, and the like.

In some embodiments, exemplary anticancer agents suitable for use in compositions and methods of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., cammustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycofommycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), nonsteroidal anti-estrogens (e.g. tamoxifen), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, (e.g. Erbitux, Rituxin, Avastin etc); 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; 23) inhibitors of protein kinases (e.g. Gleevec), and 23) radiation.

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 2 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 2

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and |
| (IgG1κ anti CD52 antibody) | | ILEX Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol | Zyloprim | GlaxoSmithKline, |
| (1,5-dihydro-4 H-pyrazolo[3,4-d]pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2,4, | | Conshohocken, PA |
| 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′-tetramethyl- | | Pharmaceuticals, LP, |
| 5-(1H-1,2,4-triazol-1-ylmethyl)) | | Wilmington, DE |
| Arsenic trioxide | Trisenox | Cell Therapeutic, |
| | | Inc., Seattle, WA |
| Asparaginase | Elspar | Merck & Co., Inc., |
| (L-asparagine amidohydrolase, type EC-2) | | Whitehouse Station, |
| | | NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| [BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| bexarotene gel | Targretin | Ligand |
| | | Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle |
| (as 4-[5-(4-methylphenyl)-3-(trifluoromethyl)- | | Pharmaceuticals, |
| 1H-pyrazol-1-yl] | | England |
| benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson |
| (2-chloro-2′-deoxy-b-D-adenosine) | | Pharmaceutical |
| | | Research Institute, |
| | | Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye |
| | | Pharmaceuticals, Inc., |
| | | San Diego, CA |
| Dacarbazine | DTIC-Dome | Bayer AG, |
| (5-(3,3-dimethyl-1-triazeno)-imidazole-4- | | Leverkusen, |
| carboxamide (DTIC)) | | Germany |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-[(3-amino-2,3,6-trideoxy-á- | | Pharmaceuticals, Inc., |
| L-lyxo-hexopyranosyl)oxy]-7,8,9,10-tetrahydro- | | Boulder, CO |
| 6,8,11-trihydroxy-1-methoxy-5,12- | | |
| naphthacenedione hydrochloride) | | |
| Daunorubicin HCl, daunomycin | Cerubidine | Wyeth Ayerst, |
| ((1 S,3 S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one 4- | | |
| acetate 2-benzoate, trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia & Upjohn |
| | Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10-tetrahydro- | | |
| 6,8,11-trihydroxy-8-(hydroxyacetyl)-1-methoxy- | | |
| 5,12-naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, |
| agent vidarabine, 9-b-D-arabinofuranosyladenine | | NJ |
| (ara-A)) | | |
| Fluorouracil, 5-FU | Adrucil | ICN Pharmaceuticals, |
| (5-fluoro-2,4(1H,3H)-pyrimidinedione) | | Inc., Humacao, |
| | | Puerto Rico |
| Fulvestrant | Faslodex | IPR Pharmaceuticals, |
| (7-alpha-[9-(4,4,5,5,5-penta fluoropentylsulphinyl) | | Guayama, Puerto |
| nonyl]estra-1,3,5-(10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D-Ser(But)⁶,Azgly¹⁰]LHRH; pyro- | | Pharmaceuticals |
| Glu-His-Trp-Ser-Tyr-D-Ser(But)-Leu-Arg-Pro- | | |
| Azgly-NH2 acetate [C₅₉H₈₄N₁₈O₁₄•(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan [N- | | |
| [2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino- | | Company |
| 2,3,6-trideoxy-(alpha)-L-lyxo- | | |
| hexopyranosyl)oxy]-7,8,9,10-tetrahydro-6,9,11- | | |
| trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4-methyl- | | Switzerland |
| 3-[[4-(3-pyridinyl)-2-pyrimidinyl]amino]- | | |
| phenyl]benzamide methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′,4′: | | |
| 6,7] indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5,6-tetrahydro-6-phenylimidazo [2,1- | | Foundation, |
| b] thiazole monohydrochloride C₁₁H₁₂N₂S•HCl) | | Titusville, NJ |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., |
| | | Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, |
| | | Germany |
| Oprelvekin | Neumega | Genetics Institute, |
| (IL-11) | | Inc., Alexandria, VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| benzoate 13-ester with (2R,3 S)-N-benzoyl-3- | | |
| phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) | (Pegademase | Pharmaceuticals, Inc., |
| 11-17-adenosine deaminase) | Bovine) | Bridgewater, NJ |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San Francisco, |
| | | CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)- | | |
| D-glucopyranose and 220 mg citric acid | | |
| anhydrous) | | |
| Talc | Sclerosol | Bryan, Corp., |
| (Mg₃Si₄O₁₀ (OH)₂) | | Woburn, MA |
| Tamoxifen | Nolvadex | AstraZeneca |
| ((Z)2-[4-(1,2-diphenyl-1-butenyl) phenoxy]-N,N- | | Pharmaceuticals |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien- | | |
| 17-oic acid [dgr]-lactone) | | |
| Thioguanine, 6-TG | Thioguanine | GlaxoSmithKline |
| (2-amino-1,7-dihydro-6 H-purine-6-thione) | | |
| Thiotepa | Thioplex | Immunex |
| (Aziridine, 1,1′,1″-phosphinothioylidynetris-, or | | Corporation |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′: 6,7] indolizino [1,2-b] | | |
| quinoline-3,14-(4H,12H)-dione | | |
| monohydrochloride) | | |
| Toremifene | Fareston | Roberts |
| (2-(p-[(Z)-4-chloro-1,2-diphenyl-1-butenyl]- | | Pharmaceutical |
| phenoxy)-N,N-dimethylethylamine citrate (1:1)) | | Corp., Eatontown, NJ |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2-oxoethyl | | |
| pentanoate) | | |
| Vinblastine, Leurocristine | Velban | Eli Lilly |
| (C₄₆H₅₆O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

In other embodiments, other agents (e.g., immunomodulatory agents, anti-inflammatory agents, NSAID, and immunotherapeutics) are administered along with a composition of the present invention.

Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, pirprofen, carprofen, oxaprozin, pramoprofen, muroprofen, trioxaprofen, suprofen, aminoprofen, tiaprofenic acid, fluprofen, bucloxic acid, indomethacin, sulindac, tolmefin, zomepirac, fiopinac, zidometacin, acemetacin, fentiazac, clidanac, oxpinac, mefenamio acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophenol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthatrazone); and alkanones, including nabumetone and pharmaceutically acceptable salts thereof and mixtures thereof. For a more detailed description of the NSAIDs, see Paul A. Insel, Analgesic-Antipyretic and Anti-inflammatory Agents and Drugs Employed in the Treatment of Gout, in Goodman & Gilman's The Pharmacological Basis of Therapeutics 617-57 (Perry B. Molinhoff and Raymond W. Ruddon eds., 19th ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

Other Examples of prophylactic and therapeutic agents include, but are not limited to, immunomodulatory agents, anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, non-steroidal anti-inflammatory drugs (e.g., aspirin, ibuprofen, diclofenac, and COX-2 inhibitors), and leukotriene antagonists (e.g., montelukast, methyl xanthines, zafirlukast, and zileuton), beta2-agonists (e.g., albuterol, biterol, fenoterol, isoetharine, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythromycin, penicillin, mithramycin, and anthramycin (AMC)).

Any immunomodulatory agent well-known to one of skill in the art may be used in the co-administration methods and compositions of the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities. In a specific embodiment of the invention, the immunomodulatory agent inhibits or suppresses the immune response in a subject.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, inorganic compounds, auto-antigens, allergens, allo-antigens, and pathogenic antigens. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, B cell receptor modulators, antigen presenting cell modulators, cytokine receptor modulators, antigens, and mast cell modulators.

Examples of T cell receptor modulators include, but are not limited to, anti-T cell receptor antibodies (e.g., anti-CD4 antibodies (e.g., cM-T412 (Boeringer), IDEC-CE9.1 (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD1 a antibodies (e.g., Xanelim (Genentech)), and anti-B7 antibodies (e.g., IDEC-114)(IDEC))), CTLA4-immunoglobulin, LFA-3TIP (Biogen, International Publication No. WO 93/08656 and U.S. Pat. No. 6,162,432), anti-CD28, anti-PD1, anti-BTLA, C-type lectin antibodies, cytokines (e.g. IL2, IFN-γ, GM-CSF, TNF, IL15, IL7, IL17).

Examples of B cell modulators include, but are not limited to, anti-IgM, anti-IgG, anti-IgD, anti-IgA, anti-IgE, anti-CD20, anti-CD20, anti-CD19, anti-CD21, anti-CD23, anti-CD30, anti-TLR9, anti-Fas, anti-Blys receptor, anti-April receptor, anti-BCMA receptor, anti-Fcgamma receptor, anti-Blys (Baff), anti-April, anti-BCMA, and anti-BTLA.

Examples of antigen presenting cell modulators include, but are not limited to, anti-CD40, anti-TLRs, antibodies to C-type lectin-like molecules (e.g. NKRP1f, OCILRP2), cytokines (e.g. GM-CSF, TNF, IL1, IL6, IL12, IL15, IL23, IL27), and antibodies to costimularoty or co-repressor molecules (e.g. CD80, CD86, PDL1, PDL2, B7-H1, B7-H3).

Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin IL-2, IL-3, IL-4, IL-5, IL-6, IL-7, IL-8, IL-9, IL-10, IL-11, IL-13, IL-15, IL-23, TNF-α, TNF-β, interferon (IFN)-α, IFN-β, IFN-7, and GM-CSF), anti-cytokine receptor antibodies (e.g., anti-IFN receptor antibodies, anti-IL-2 receptor antibodies (e.g., Zenapax (Protein Design Labs)), anti-IL-3 receptor antibodies, anti-IL4 receptor antibodies, anti-IL-6 receptor antibodies, anti-IL-10 receptor antibodies, anti-IL-12 receptor antibodies, anti-IL-13 receptor antibodies, anti-IL-15 receptor antibodies, and anti-IL-23 receptor antibodies), and anti-cytokine antibodies (e.g. anti-TNF, anti-IL2, anti-IL6).

In one embodiment, an antigen is a self- or auto-antigen, allergen, foreign- or allo-antigen, or pathogenic antigen. Example of self and allo-antigen include, but not limited to, insulin, extract or cells derived from insulin-producing beta cells, collagen, extract or cells derived from synoviocytes, myelin basic protein (MBP), glycoproteins derived from neuronal tissues, MHC-mis-matched donor cells, tissues, extracts or MHC-complexes.

Example of allergens and pathogenic antigens include molecules or extracts derived from pollens, dust mite, pathogenic bacteria or viruses (e.g., M. tuberculosis, HCV, HIV, Herpes simplex, Helicobacter pylori, Listeria monocytogeneses, streptococcus, influenza virus, bird flu virus (H5N1), SARS coronavirus, HCV, HIV, EBV, Herpes simplex, Helicobacter pylori, Listeria).

In one embodiment, a cytokine receptor modulator is a mast cell modulator. Examples of mast cell modulators include, but are not limited to stem cell factor (c-kit receptor ligand) inhibitor (e.g., mAb 7H6, mAb 8H7a, pAb 1337, FK506, CsA, dexanthasone, and fluconcinonide), c-kit receptor inhibitor (e.g., STI 571 (formerly known as CGP 57148B)), mast cell protease inhibitor (e.g., GW-45, GW-58, wortmannin, LY 294002, calphostin C, cytochalasin D, gertistein, KT5926, staurosporine, and lactoferrin), relaxin (“RLX”), IgE antagonist (e.g., antibodies rhuMAb-E25 omalizumab, HMK-12 and 6HD5, and mAB Hu-901), IL-3 antagonists, IL-4 antagonists, IL-10 antagonists, and TGFbeta.

In combination therapy treatment, both the compounds of this invention and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment of the invention where another therapeutic agent is administered to an animal, the effective amount of the compound of this invention is less than its effective amount would be where the other therapeutic agent is not administered. In another embodiment, the effective amount of the conventional agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In various embodiments, the therapies (e.g., prophylactic or therapeutic agents) are administered less than 5 minutes apart, less than 30 minutes apart, 1 hour apart, at about 1 hour apart, at about to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patent visit.

In certain embodiments, one or more compounds of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same compounds of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than a compound of the invention may be repeated and the administration may be separated by at least at least day, 2 days, 3 days, 5 days, 10 days, 15 days, 30 days, 45 days, 2 months, 75 days, 3 months, or at least 6 months.

### II. Antibodies

The present invention provides isolated antibodies. In preferred embodiments, the present invention provides monoclonal antibodies that specifically bind to an isolated polypeptide comprised of at least five amino acid residues of c-Rel. These antibodies find use in the therapeutic and research methods described herein. In some embodiments, antibodies also find use in research applications, drug screening, and therapeutic applications (e.g., antibodies directed to factors that influence c-Rel signaling).

An antibody against a protein of the present invention may be any monoclonal or polyclonal antibody, as long as it can recognize the protein. The present invention further contemplates intrabodies, recombinantly produced antibodies, human antibodies, humanized antibodies, chimeric antibodies, synthetic antibodies, single-chain Fvs antibodies, single chain antibody Fab fragments, F(ab′) fragments, disulfide-linked Fvs antibodies, anti-idiotypic antibodies, and epitope binding fragments of any of the above. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant or other adjuvant (e.g., mineral gels such as aluminum hydroxide, surface active substances such as lysolecithin, pluronic polyols, polyanions, peptides, oil emulsions, dinitrophenol, and other adjuvants such as bacilli Calmette-Guerin and Corynebacterium parvum) may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against c-Rel). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against c-Rel) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, c-Rel protein (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

In some embodiments, antibodies (e.g., monoclonal antibodies) are humanized. Humanized antibodies are altered in order to make them less immunogenic to humans, e.g., by constructing chimeric antibodies in which a mouse antigen-binding variable domain is coupled to a human constant domain. Humanized antibodies are typically human antibodies in which some CDR residues and possibly some FR residues are substituted by residues from analogous sites in rodent antibodies. Methods for humanizing antibodies are well known in the art and include but are not limited to, those disclosed in U.S. Pat. Nos. 6,054,297, 4,816,567, 6,180,377, 5,871,907, 5,585,089, and 6,180,370, each of which is herein incorporated by reference.

In other embodiments, techniques described for the production of single chain antibodies (See e.g., U.S. Pat. No. 4,946,778, herein incorporated by reference in its entirety) can be adapted to produce c-Rel specific antibodies. An additional embodiment utilizes the techniques described for the construction of Fab expression libraries (Huse et al., 1989, Science 246:1275, herein incorporated by reference in its entirety).

The present invention further contemplates human antibodies. Human antibodies may be obtained by using human hybridomas (Cote et al., 1983, PNAS 80:2026, herein incorporated by reference in its entirety), by transforming human B cells with EBV virus in vitro (Cole et al., 1985 In: Monoclonal Antibodies and Cancer Therapy, Alan R. Liss, Inc., pp. 77-96), or by immunizing transgenic mice that are engineered to replace mouse Ig gene loci with that of human origin. In other embodiments, techniques useful for the production of chimeric antibodies (e.g., Morrison et al., 1984, PNAS 81:6851; Neuberger et al., 1984, Nature 312:604; Takeda et al., 1985, Nature 314:452, each of which is herein incorporated by reference in its entirety) by splicing the genes from a mouse antibody molecule specific for c-Rel together with genes from a human antibody molecule of appropriate biological activity.

### III. Drug Screening

In some embodiments, the present invention provides drug screening assays (e.g., to screen for anticancer or anti-inflammatory drugs). In some embodiments, the screening methods of the present invention utilize c-Rel. For example, in some embodiments, the present invention provides methods of screening for compounds that alter (e.g., decrease) the expression of c-Rel. In other embodiments, candidate compounds are antisense, siRNA agents (e.g., oligonucleotides), aptamers, or peptides directed against c-Rel. In still further embodiments, candidate compounds are small molecules or natural compounds that inhibit the activity of c-Rel. Exemplary assays for screening candidate inhibitors are described for example, in the Experimental section below.

In some preferred embodiments, the drug screening assays described in the Experimental sections below are used to evaluate candidate compounds. For example, in some embodiments, a fluorescence polarization (FP)-based high-throughput screening assay is used to identify small molecules that disrupt the binding of human c-Rel homodimer to a binding partner (e.g., CD28 response element (CD28RE) in the promoter region of IL-2 gene). In some embodiments, compounds identified in the fluorescence polarization assay are further screened using an electrophoretic mobility shift assay. However, the present invention is not limited to the methods disclosed below. Other drug screening methods are contemplated to be within the scope of the present invention.

In one screening method, candidate compounds are evaluated for their ability to alter (e.g., decrease) c-Rel activity by contacting a compound with a cell expressing c-Rel and then assaying for the affect of the candidate compounds on c-Rel function or expression. In some embodiments, the affect of candidate compounds on activity of c-Rel is assayed for by detecting a decreasing level of c-Rel in the nuclear extract by electrophoretic mobility shift assay or immunoblotting. In other embodiments, the effect of candidate compounds is assayed by measuring the level of c-Rel biological activity in regulating c-Rel downstream target genes (e.g. cytokines, cell cycle proteins, cell survival proteins and other target genes described herein). The level of c-Rel target gene mRNA expression can be measured using any suitable method, including but not limited to, those disclosed herein such as Northern blotting, quantitative PCR, microarray, or by monitoring a phenotype (e.g., reduction in symptoms or cancer, inflammatory or autoimmune disease).

In some embodiments, in vitro drug screens are performed using purified wild type or subdomain of c-Rel and binding partners thereof. Compounds are screened for their ability to interact with c-Rel proteins and inhibit c-Rel function or the interaction of c-Rel with binding partners. In some embodiments, binding partners are immobilized to facilitate separation of complexed from uncomplexed forms of one or both of the proteins, as well as to accommodate automation of the assay. Binding of a test compound to c-Rel or its binding partners (e.g., DNA, p300/CBP, co-activator, mediator, or other transcription factor including STAT3, STATS, c-Jun, IRF) is accomplished in any vessel suitable for containing the reactants. Examples of such vessels include microtitre plates, test tubes, and microcentrifuge tubes. In one embodiment, a fusion protein can be provided which adds a domain that allows one or both of the proteins to be bound to a matrix. For example, glutathione-S-transferase/AIP-6 fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and the non-adsorbed protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly. Alternatively, the complexes can be dissociated from the matrix, and the level of protein binding or activity determined using standard techniques.

Other techniques for immobilizing proteins on matrices can also be used in the screening assays of the invention. For example, upstream c-Rel signaling proteins (e.g. PI3K, PKC-theta, IKK) or other protein known to interact with or modulate signaling by c-Rel can be immobilized utilizing conjugation of biofin and streptavidin. Biotinylated proteins are prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with c-Rel signaling proteins but which do not interfere with binding of the protein to test compounds can be derivatized to the wells of the plate, and unbound protein trapped in the wells by antibody conjugation. Methods for detecting such complexes, in addition to those described above for the GST-immobilized complexes, include immunodetection of complexes using antibodies reactive with c-Rel signaling proteins, as well as enzyme-linked assays that rely on detecting an enzymatic activity associated with c-Rel signaling.

In other embodiments, a competitive drug screening assays in which neutralizing antibodies capable of binding c-Rel specifically compete with a test compound for binding to c-Rel are utilized. In this manner, the antibodies can be used to detect the presence of any compound that shares one or more antigenic determinants with c-Rel.

In still further embodiments, transgenic animals having altered (e.g., inactivated or overexpressed) c-Rel genes are utilized in drug screening applications. For example, in some embodiments, compounds are screened for their ability to reduce metastasis or inflammation in c-Rel transgenic or knockout mice.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckermann et al. J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. EngI. 33.2059 [1994]; Carell et al., Angew. Chem. Int. Ed. Engl. 33:2061 [1994]; and Gallop et al., J. Med. Chem. 37:1233 [1994].

Libraries of compounds or peptides may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In other embodiments, fluorescence resonance energy transfer (FRET), computational modeling base on structural information obtained from X-ray crystallography, gene-expression profiling-based high throughput drug screening (GE-HTS) or co-immunoprecipitation (co-IP) may be used to identify or evaluate compounds with the ability to inhibit c-Rel function or interfere with the interaction of c-Rel with binding partners (e.g., co-activators, co-repressors, transcription mediators, signaling molecules, and transcription factors including c-Rel, p50, p65, RelB, p52, STATs, IRFs, c-jun, c-fos, Foxp3, and NF-ATs).

### IV. Transgenic Animals Expressing or Lacking C-Rel

The present invention contemplates the generation of transgenic animals comprising an exogenous c-Rel gene or mutants and variants thereof (e.g., truncations, deletions, insertions, single nucleotide polymorphisms, or heterologous c-Rel genes under control of a promoter that overexpresses the gene)). In other embodiments, the present invention provides transgenic animals with a knock-out of the c-Rel gene. In preferred embodiments, the transgenic animal displays an altered phenotype (e.g., increased or decreased cancer or symptoms of inflammatory or autoimmune disease) as compared to wild-type animals. Methods for analyzing the presence or absence of such phenotypes include but are not limited to, those disclosed herein.

The transgenic animals of the present invention find use in drug (e.g., cancer or inflammatory disease) screens. In some embodiments, test compounds (e.g., a drug that is suspected of being useful to treat cancer or inflammatory diseases) and control compounds (e.g., a placebo) are administered to the transgenic animals and the control animals and the effects evaluated.

In some embodiments, the transgenic animals described in the Experimental section below are utilized in drug screening applications. However, the present invention is not limited to the transgenic animals disclosed herein. Additional transgenic animals may be generated using a variety of methods including, but not limited to, those disclosed below.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present invention and are not to be construed as limiting the scope thereof.

### Example 1

### C-Rel Inhibitors

This Example describes the identification of small molecule inhibitors of c-Rel.

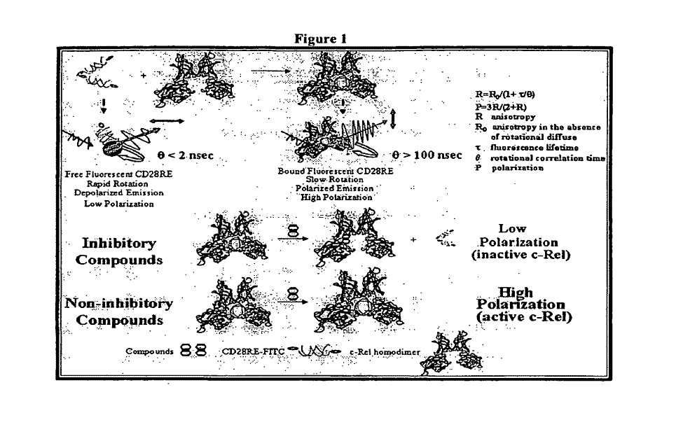

Historically, transcription factors have been considered difficult to access by small molecule inhibitors due to the large interaction surface mediating the binding of transcription factors to DNA. However, there is growing evidence in the literature and from screening initiatives to suggest that small molecules can modulate the interactions responsible for DNA-protein and protein-protein complex formation. Since c-Rel functions primarily via forming a homo- or hetero-dimer and binding its cognate DNA site in the promoter region of targeted genes, small molecules that are able to efficiently disrupt the formation of c-Rel dimer-targeted kB site complex are particularly desirable inhibitors. These compounds may act either directly via inhibition at the protein-DNA interface or dimerizational interface, or indirectly via binding to an allosteric site and induction of a conformational change of c-Rel protein. To identify such inhibitors a fluorescence polarization (FP)-based high-throughput screening assay was developed to identify small molecules that disrupt the binding of human c-Rel homodimer to the CD28 response element (CD28RE) in the promoter region of IL-2 gene (FIG. 1).

FP measurements are based on the assessment of the rotational motions of sample molecules and can be used to monitor the binding of two molecules to each other. FP can be considered a competition between the molecular motion and the lifetime of fluorophores in solution. If linear polarized light (see FIG. 1) is used to excite an ensemble of fluorophores (FIG. 1, oval shape, in the case of c-Rel, FITC) only those fluorophores aligned with the plane of polarization will be excited. There are 2 scenarios for the emission. Provided the fluorescence lifetime of the excited fluorescent probe is much longer than the rotational correlation time θ of the molecule it is bound to (θ is a parameter that describes how fast a molecule tumbles in solution), the molecules will randomize in solution during the process of emission, and, as a result, the emitted light of the fluorescent probe will be depolarized, and fluorescence anisotropy or fluorescence polarization, two mathematical descriptions that can easily be converted to one another as shown in FIG. 1, is low. If the fluorescence lifetime of the fluorophore is relatively much shorter than the rotational correlation time θ (for example, θ dramatically increases after CD28RE-FITC is bound to c-Rel protein), the excited molecules (c-Rel homodimer-CD28RE-FITC complex) will stay aligned during the process of emission, as a result, the emission will be polarized, and thus fluorescence anisotropy or fluorescence polarization is high.

Fluorescence polarization measurements have been developed for a variety of biochemical interactions, including G protein-coupled receptors, actin binding proteins, tyrosine kinases, etc. Unlike energy-transfer based read outs that require two labeled species, FP has the advantage of requiring only one labeled species for the assay and thus FP has become a very popular read out format for high throughput screening in drug discovery, including identifying small molecule inhibitors of the DNA binding of transcription factors like B-ZIP.

The human full-length or subdomain of c-Rel protein used in the FP assay was generated in E. coli cells by following the conventional protocol. Briefly, human c-Rel gene was inserted into the expression vector and transformed into E. coli BL-21 Lys (DE3). Cells were grown and inducted by IPTG. The recombinant protein containing a His tag at its C-terminal was purified by affinity chromatography using Nickel resin according to the manufacture's instruction (Qiaqen).

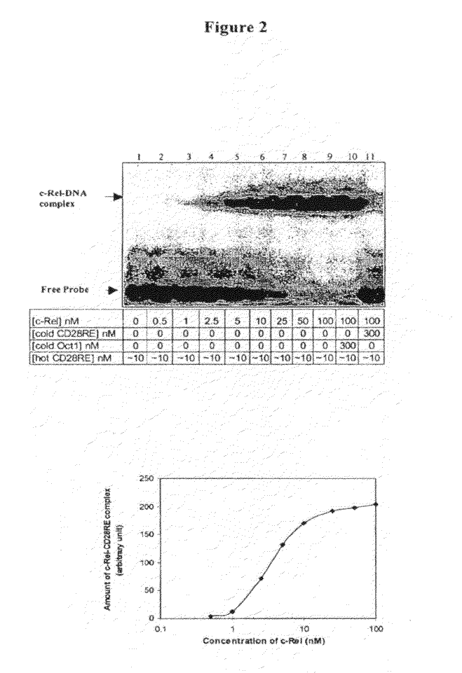

To evaluate and confirm the functional equality of the purified human c-Rel protein, a conventional electrophoretic mobility shift assay (EMSA) was performed to determine the specific interaction between human c-Rel and its targeted kB site CD28RE (FIG. 2). In this EMSA experiment, the concentration of ³²P labeled CD28RE DNA probe was held constant (1˜100 nM) in each lane and titrated with increasing c-Rel concentrations. The data best fit a cooperative binding model describing two subunits assembling sequentially (FIG. 2, left panel, lane 1-9 and right panel). Furthermore, the cold competition experiments demonstrated that the unlabeled CD28RE at 300 nM is able to totally block the formation of c-Rel homodimer-³²P-ATP labeled CD28RE complex (FIG. 2, lane 11) but the unlabeled Oct1 as a control oligo at the same level has no inhibitory effect on the DNA binding activity of human c-Rel (FIG. 2, lane 10), demonstrating that the observed DNA-protein complex is due to the specific interaction between human c-Rel and its targeted kB site CD28RE.

For the FP experiments, the sequence of the 20-mer kB site from the CD28 RE (underlined) was as follow: 5′-FITC-TCTGGAATTTCTTTAAACCC-3′ (SEQ ID NO:37) (ordered from Biosource and HPLC purified).

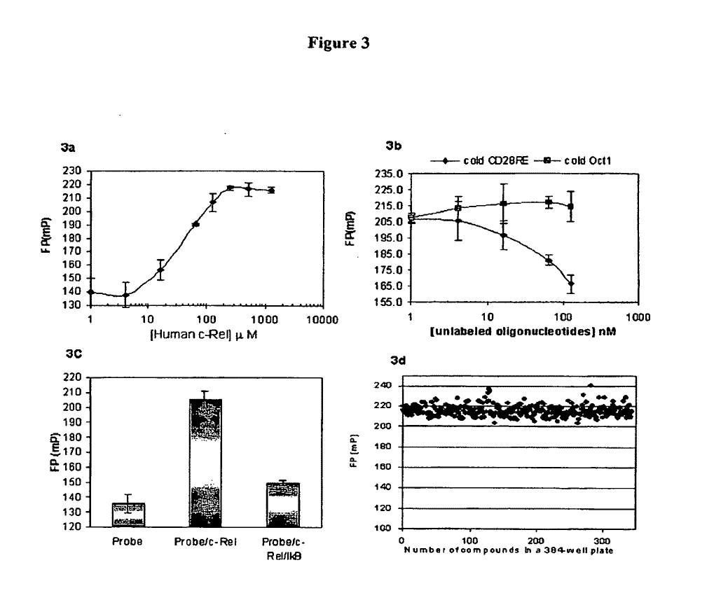

According to the EMSA data, the concentrations of FITC-labeled DNA probe and c-Rel protein used in FP screen assay were optimized. Initially, the fluorescently labeled probe was added in different concentrations (5 nM, 10 nM, 100 nM) while c-Rel protein concentration (2000 nM) was constant, and the reactions were incubated for 15-30 minutes at room temperature, and then transferred to the wells of 384-well plate (20 μl/well). The FP values were measured using FUSION Universal Microplate Analyzer (Perkin Elmer, PE). The data indicated that 10 nM of the probe is the better choice for generating the c-Rel protein-induced FP difference between the DNA probe alone and the DNA-protein complex. Consistent with the data derived from EMSA, titration of increasing concentrations of c-Rel protein binding to 10 nM of the fluorescent DNA probe resulted in two phases of increasing FP, with steeper increase at lower concentrations, followed by shallower increase at higher concentration (FIG. 3a). In most cases, there is an increase in FP, with FP signals saturated in the presence of 128 nM of c-Rel. The dissociation constant (Kd) calculated using this method is larger than that observed using EMSA.

To exclude the possibility that non-specific interaction of c-Rel protein with DNA molecules contributes to the increase in FP value, a cold competition experiment was performed. As shown in FIG. 3b, titration of the unlabeled CD28RE into the reactions containing the constant amount of c-Rel and the fluorescent DNA probe led to a gradual decrease in FP value, while returning to the basal level of the fluorescent probe at the highest concentration tested (128 nM). However, a control DNA molecule, the unlabeled Oct1 (20 mers) in the same conditions as applied to the cold CD28RE probe, had no effect on FP value. Thus, this assay specifically and quantitatively measured interactions in the interface between c-Rel protein and CD28 RE.

It was next investigated whether this assay could detect a known endogenous inhibitor (IkBα) of c-Rel activity. Previous structural studies have demonstrated that upon binding to NF-κB, IkBα promotes a large en bloc movement of the NF-κB subunit amino-terminus and allosterically inhibits NF-κB DNA binding by inducing a conformational change. Recombinant GST-IkBα previously generated (final concentration is 150 nM) was added to a binding reaction consisting of 10 nM FITC-CD28RE and 100 nM c-Rel. FP from the protein-DNA interaction was completely inhibited (FIG. 3c). Thus, this assay detected specific inhibitors that function by directly binding c-Rel protein and inducing a conformation change. Moreover, consistent with the scatter data distribution shown in FIG. 3d, z factor, one assay performance indicator, was also evaluated. In the majority of the 384-well plates tested to date, the Z factor is more than 0.5 (0.7-0.8), indicative of a statistically significant assay.

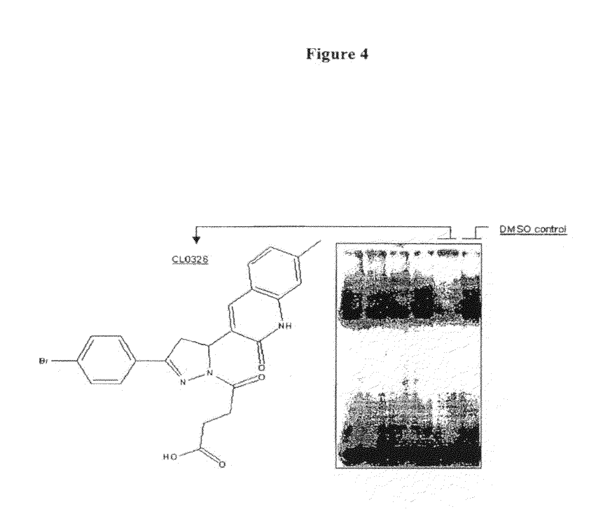

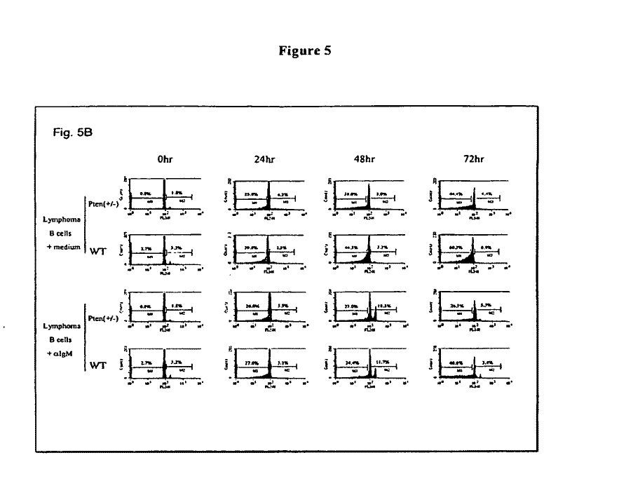

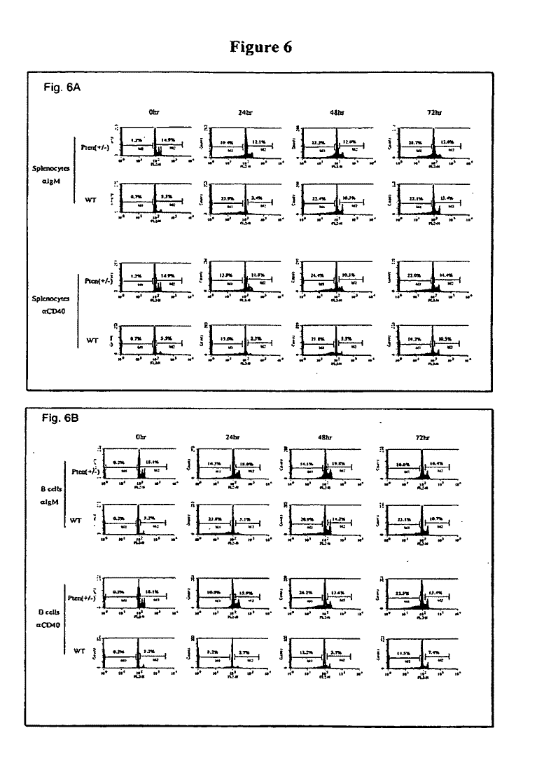

Using the above described high-throughput assay, libraries of small molecules were screened for inhibition of the interaction between c-Rel protein and the targeted KB site CD28RE. One library of 16,000 compounds was screened, yielding 100 compounds that inhibited FP signals by more than 45%. These compounds were further tested in EMSA, which identified at least 19 compounds that significantly suppressed c-Rel DNA binding activity. FIG. 4 presents a sample hit.

### Example 2

### Additional Optimization Assays

This Example describes methods for screening and optimizing c-Rel inhibitors.

### Measure K_{D} (Binding Affinity)

In some embodiments, K_{D}-binding affinity is measured (e.g., using biocore). DNA is immobilized on a plate and protein is contacted with DNA to measure affinity (K_{D}) in relation to hit compounds. Since non-specific competitor nucleic acids such as poly (dI:dC) were included in excess in the high throughput assay, it is unlikely that the hit compounds bind to DNA structure non-discriminately.

### Chemical Modification of Lead Compounds

In some embodiments, chemical modifications are performed to improve affinity and cell permeability. In some embodiments, functional groups are varied to correlate with EC50 to identify functional groups that are or are not involved in activity and to identify which piece of the molecules are crucial for activity and which pieces are available for manipulation to enhance other properties such as permeability. Charged molecules can have difficulty entering cells. In some embodiments, lead compounds are modified as uncharged molecules (pro-drugs) that can be converted into active compounds after entering cells. For example, in some embodiments, molecules are derivatized with ester groups that can be cleaved by endogenous esterase activity in cells. Stereoisomers of the lead compounds are also analyzed for activity. The effect of compounds on inhibiting c-Rel activity is tested using the above in vitro and cellular assays. The effect of lead compounds on inhibiting c-Rel activity in cells (reporter assay, apoptosis assay), as well as EC50 values are also measured.

Lead Compounds with optimal biochemical, physicochemical, and cellular properties that meet certain criteria (e.g. 5× or 10× lower EC50 than the original hits) are further optimized, or fine-tuned.

### Example 3

### Effect of c-Rel Blockade on Lymphoma

This Example demonstrates that c-Rel blockade attenuates hyper-proliferation of lymphoma.

### A. Materials and Methods

### Mouse Strains

Pten(+/−) mice and Pten^{flox/+} mice (background strain C57/BL6) were provided by Dr. Pandolfi PP (Di Cristofano et al., 1999 Science 285:2122). To generate B cell-specific Pten-deficient mice, Pten^{flox/+} mice were crossed with CD19^{cre/+} transgenic mice (purchased from Jackson lab). Offspring carrying CD19^{cre/+} and the floxed Pten mutation on both alleles (Pten^{flox/flox} CD19^{Cre/+}), and the wild-type Pten gene (Pten^{+/+} CD19^{Cre/+}) were used for analysis as homozygous mutant, wild-type mice, respectively. The mice were maintained under specific pathogen-free conditions in the animal colony of Weill Medical college of Cornell University. Pten^{flox/flox} CD19^{Cre/+} mice were crossed with c-Rel-deficient mice (Tumang et al., 1998 Eur J Immunol 28:4299) to generate the double deficient mice.

### Proliferation and Survival Assays

For propidium iodide staining assay, B-cells were cultured in 10 μg/ml anti-IgM or 10 μg/ml anti-CD40 or 10 μg/ml LPS. At the indicated time points, cells were collected and stained with a solution containing 50 μg/ml propidium iodide, 20 ng/ml RNase A, 0.1% Triton X-100, and 0.1% sodium citrate. Duplicate samples were then analyzed on a Becton-Dickinson FACS using Cell-Quest software, and the percentage of apoptotic cells (<2N DNA content) or S and G2/M phase cells (>2N DNA content) quantified. For ³H-thymidine incorporation assays, B-cells were plated at 1×10⁵ cells per well in 96-well U-bottomed plates and cultured in complete media containing RPMI 1640, 10% heat inactivated FCS (DeWned; Hyclone, Logan, Utah), 2 mM L-glutamine, 1 mM non-essential amino acids, 100 μg/ml, penicillin/streptomycin, and 5×10⁻⁵ M/L⁻¹ mercaptoethanol at 37° C. and 5% CO₂. Where indicated, cells were stimulated with 10 μg/ml goat anti-mouse IgM F(ab′)2 (anti-IgM, Jackson ImmunoResearch Laboratories), or 10 μg/ml anti-CD40 (mAb 1C 10), or 10 μg/ml LPS. Six hours prior to the indicated time points, cultures were incubated with 0.5 μCi/well thymidine (Amersham), and then harvested onto Whatman Filters to determine proliferative responses by thymidine incorporation. All assays were performed in triplicate, and SEM values indicated in each experiment.

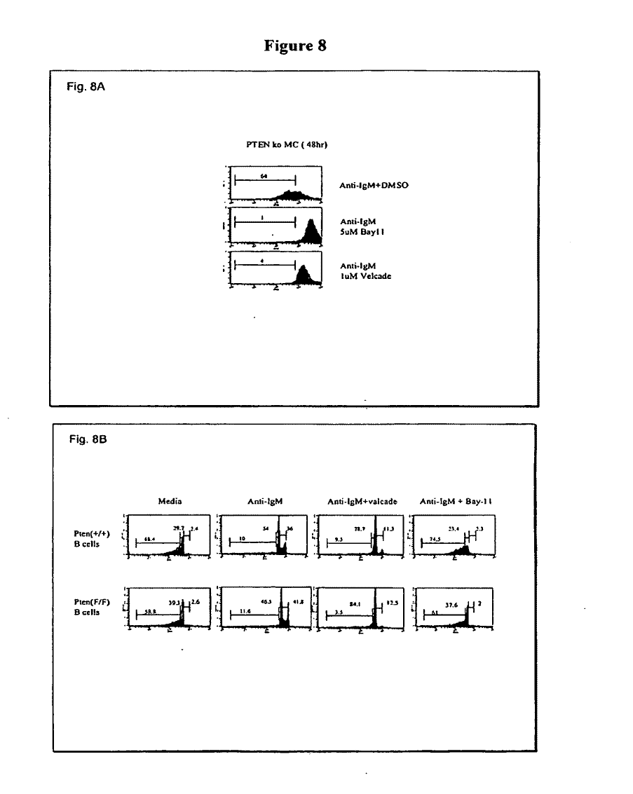

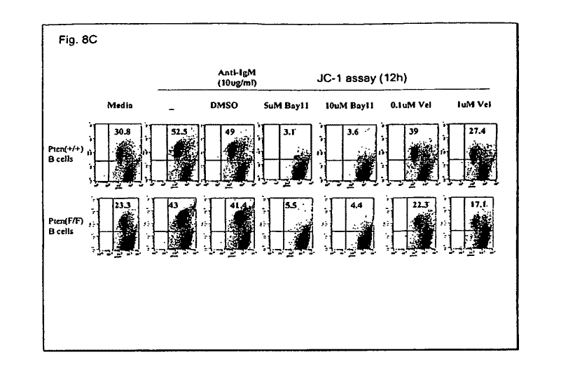

For CFSE staining, a total of 2×10⁶ purified splenic B cells were cultured in 1 ml complete media mixed with 1 ml of 5 mm CFSE (Molecular Probes) dissolved in DMSO, and incubated for 10 min at 37° C. After washing twice with complete media, 2×10⁵ B cells were cultured per well in 96-well plates and stimulated with 10 mg/ml anti-IgM in the presence or absence of various NF-kB inhibitors as indicated in FIG. 8A. At the indicated time points, cells were collected and the cell cycle analysis were performed on a FACS calibur machine using CellQuest software (Becton Dickinson).

### Measurement of Mitochondrial Potential

B cells were cultured to a density of 0.5−1.0×10⁶ cells/ml. For each condition, 1 ml of cells was treated with the appropriate agent for the appropriate length of time as shown in FIG. 8C. Mitochondrial potential was assessed by using the fluorescent potentiometric dye JC-1 (5,5′,6,6′,-tetrachloro-1,1′,3,3′-tetraethylbenzimidazolylcarbocyanine iodide, Molecular Probe). JC-1 is a novel cationic carbocyanine dye that accumulates in mitochondria. The dye exists as a monomer at low concentrations and yields green fluorescence, similar to fluorescein. At higher concentrations, the dye forms J-aggregates that exhibit a broad excitation spectrum and an emission maximum at approximately 590 nm, similar to PE. These characteristics make JC-1 a sensitive marker for mitochondrial membrane potential. Briefly, 0.3 ml of the cells were mixed with 0.3 ml of staining solution (complete medium containing 0.5 μg/1 ml JC-1). Cells were stained for 30 min in a 37° C. incubator (5% CO₂). After staining, cells were collected at room temperature and washed once in 1×PBS. The cell pellet was then resuspended in PBS (pre-warmed to 37° C.), and JC-1 fluorescence was analyzed on a Becton-Dickinson FACS using Cell-Quest software.

### Electrophoretic Mobility Shift Assay

Cells were cultured in the presence or absence of 10 μg/ml anti-IgM or control media. At the indicated time points, cells were harvested and lysed in Buffer C (20 mM Hepes, pH 7.6, 1.5 mM MgCl₂, 0.42 M KCl, 25% glycerol, 0.1% NP-40, 0.2 mM EDTA, 1 mM PMSF, 1 mM DTT, 5 μg/ml pepstatin, 5 μg/ml leupeptin, 5 μg/ml aprotinin, 4 mM NaF, and 4 mM sodium vanadate), and sonicated with 20 pulses at 4° C. Samples were then centrifuged at 13,000×g for 10 min to pellet debris, which was then discarded. Radiolabeled probes were generated by annealing 200 ng of the following single-stranded IgkB loop oligo sequence 5′-GAGAGGGGACTTTCCGATTAGCTTTCGGAAAGTCCCCTC-3′ (SEQ ID NO:1). Probes were end-labeled with 5 μCi [γ-32P]ATP. For the binding reaction, 20 μg of each nuclear extract sample was incubated for 20 min on ice with the following: 40,000 cpm of radiolabeled probe in 10 mM Tris-HCl, pH 7.5, 1 mM DTT, 1 mM EDTA, pH 7.5, 5% glycerol, 0.1 μg/μl poly(dl-dC) (Boehringer Mannheim), and 0.5% NP-40. Buffer C was used to balance the reaction to a final concentration of 200 mM KCl in a total volume of 20 μl. For cold competition, 10× or 50× excess of unlabeled IgkB was pre-incubated with the nuclear extracts for 10 min before adding ³²P-labeled IgkB probe. Reaction samples were resolved on a 6% polyacrylamide gel in 0.25 L Tris-borate-EDTA buffer for 3 h at 160V, dried onto filter paper (Whatman), and exposed to film overnight.

### RT-PCR (EBI3)

For RT-PCR analysis of EBI3 mRNA expression in pten deficient splenic lymphocytes and lymphoma cells, total RNA was isolated from these cells purified from mice with lymphoma or wild type B cells as control, respectively, using STAT-60 RNA isolation reagent following manufacturer's instruction (TEL-TEST, Friendswood, Tex.) and was PCR amplified after reverse transcription into 1st strand cDNA. The PCR condition were: 30 cycles, 94° C., 15 s; 60° C., 15 s; 72° C., 30 s. The primers used for RT-PCR were: EBI3 FP1 (19 nt): GTG CAA TGC CAT GCT TCT C (SEQ ID NO:2) (position 316); EBI3 RP1 (19 nt): TGC CAC CCT CAA GTA GAC G (SEQ ID NO:3) (position 945). The expected PCR product for EBI3 FP1/EBI3 RP1 from mouse cDNA is 648 bp. PCR products were separated by 1% agarose gel electrophoresis and visualized by UV irradiation after ethidium bromide staining.

B-cells were cultured in 10 μg/nl anti-IgM or 10 μg/ml anti-CD40. At the indicated time points, cells were collected and stained with a solution containing 50 μg/ml propidium iodide, 20 ng/ml RNase A, 0.1% Triton X-100, and 0.1% sodium citrate. Duplicate samples were then analyzed on a Becton-Dickinson FACS using Cell-Quest software, and the percentage of apoptotic cells (<2N DNA content) or S and G2/M phase cells (>2N DNA content) quantified.

### B. Results

### Pten-Mutant Lymphoma and B Cells Exhibit Accelerated Proliferation

To investigate the hyper-proliferative, autoimmune, and tumorigenic nature of Pten mutant cells, three strains of Pten knockout mice were used: 1), heterozygotic Pten(+/−) strain, 2), CD19^{cre/+} Pten^{F/F} strain derived from Pten^{F/F} (or flox/flox) mice bred to the Cre-transgenic mouse under the control of the CD19 promoter, 3), Pten (hypo-allele).

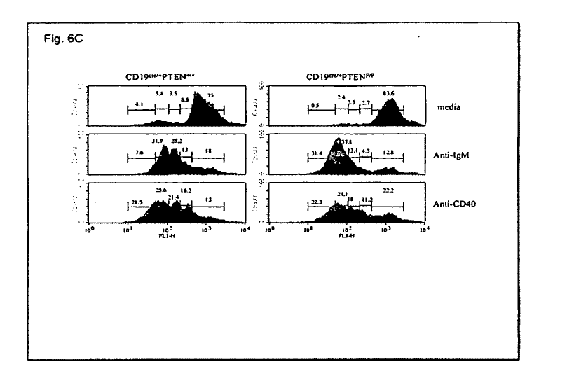

B cells derived from Pten(+/−) and CD19^{cre/-} Pten^{F/F} mice had a higher proliferative response to antigenic signals. This trend became more pronounced in 6-9 month old Pten(+/−) mice that developed islet lymphoma. Lymphoma B cells derived from Pten (+/−) mice had a higher proliferation index than normal lymph node cells in response to BCR and CD40 (FIG. 5B). Splenocytes and purified B cells from these mice also exhibited spontaneous proliferation in medium and accelerated cell cycle progression in response to BCR and CD40 signals (FIGS. 6A and 6B). Pten-mutant B cells also survived better under anti-IgM treatment (FIG. 6B). Using CFSE to monitor cell division, a that higher percentage of CD19^{cre/+} pten^{F/F} B cells progressed to three and four cell divisions than that of normal B cells (−70% vs 39%) in response to anti-IgM (FIG. 6C). Pten (+/−) T cells from spleen also had slightly higher spontaneous cell proliferation in medium alone. TCR/CD28 was able to sustain the cell cycle of these cells.

### Sustained NF-kB/Rel Activation in Pten-Mutant B Cells

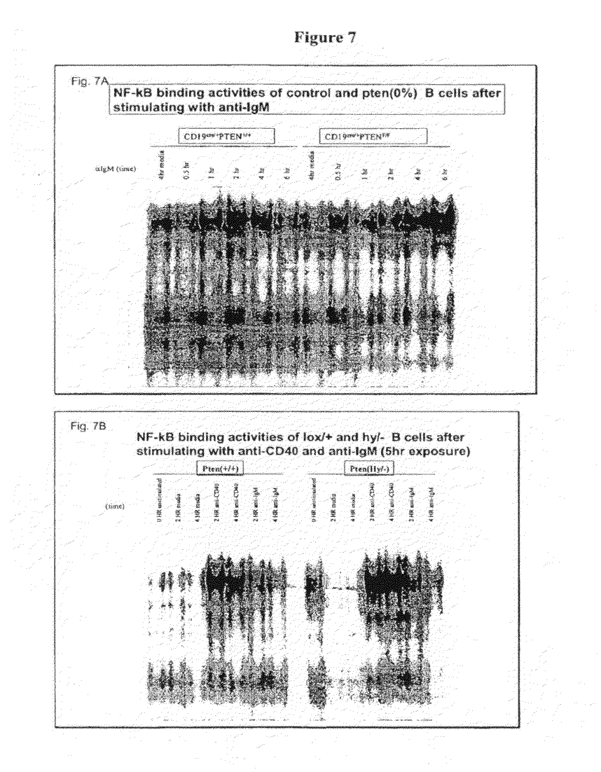

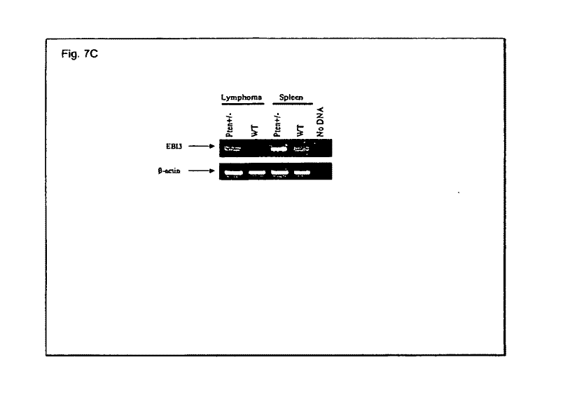

Since the hyper-proliferative nature associated with Pten-mutation is not defined, the link with NF-kB/Rel activation was investigated. Nuclear extract derived from Pten-mutant and control B cells was analyzed for nuclear NF-kB/Rel binding activity by using the EMSA (Electrophoretic Mobility Shift Assay). Control B cells exhibited a transient activation of NF-kB/Rel activity at 1 and 2 hours post stimulation with anti-IgM (FIG. 7A). In comparison, the Pten-mutant B cells revealed an early activation at 0.5 hour, and a sustained NF-kB/Rel activity throughout the 6 hour time course. Sustained NF-kB/Rel activation by anti-CD40 stimulation was also observed in B cells derived from a different strain of Pten-mutant mouse (hy/−) (FIG. 7B).

To further confirm the physiological consequence of NF-kB/Rel activation on the regulation of its downstream targets, one of the c-Rel targets, EBI3, was investigated. The data indicated that EBI3 level is substantially higher in spleen and lymphoma samples derived from the Pten(+/−) mice as compared to control tissues (FIG. 7C). These results demonstrate that Pten-mutation leads to sustained NF-kB/Rel nuclear translocation and results in upregulation of c-Rel target genes that are responsible for cell cycle and cell survival.

### NF-kB Inhibition Blocks Cell Division and Induces Apoptosis of Pten-Mutant B Cells

To further sort through the contribution of NF-kB/Rel hyper-proliferation and tumorigenesis, the pharmacological NF-kB inhibitors Bay 11 and Velcade were used. Pten-mutant cells underwent robust cell divisions after 3 days of stimulation with BCR signal. Inclusion of NF-kB inhibitors completely blocked the division of Pten-mutant cells (FIG. 8A). This observation was corroborated by DNA content analysis with propidium iodine staining (FIG. 8B). In addition to blocking cell cycle progression, Bay 11 treatment also led to significant cell apoptosis.

One of the mechanisms by which NF-kB/Rel protects cells from apoptosis is by inducing the expression of survival protein of the Bcl-2 family, including Bcl-X, Bfl-1, and Mcl-1 (Owyang et al., 2001 J Immunol 167:4948; Tumang et al., 2002 Cell Immunol 217:47). One of the key functions entailed by the Bcl-2 family is to regulate the integrity of mitochondrial membrane potential. It was contemplated that blocking NF-kB/Rel activity would affect the expression of the survival proteins, leading to mitochondrial depolarization and release of apoptotic mediators. Using JC-1 staining to assess mitochondrial membrane potential, the data indicated that both NF-kB inhibitors rapidly induced mitochondrial depolarization within 12 hours of anti-IgM treatment (FIG. 8C).

Taken together, the data suggest that blocking NF-kB/Rel suppresses Pten-mutant cell hyper-proliferation that leads to tumorigenesis.

### C-Rel Deletion Induces Apoptosis and Cell Cycle Arrest of Pten-Mutant B Cells

Since NF-kB (p50/p65 dimer) is broadly utilized by all sorts of tissues, strategies that target on pan-NF-kB members, including INK inhibitors and the two aforementioned NF-kB inhibitors, would expect to impose severe side-effects including liver toxicity. This argument is strongly supported by the embryonic lethality and liver necrosis of the p65 and IKKβ knockout mice (Beg et al., 1995 Nature 376:167; Li et al., 1999 J Exp Med 189:1839). C-Rel specific inhibition is thus a safer strategy. C-Rel function is restricted to mature lymphocytes and myeloid cells. Furthermore, c-Rel knockout mice are as physiologically viable as normal mice, except for their slightly compromised immune system (Kontgen et al., 1995 Genes & Dev. 9:1965; Liou et al., 2003 Bioessays 25:767; Liou et al., 1999 Int Immunol 11:361).

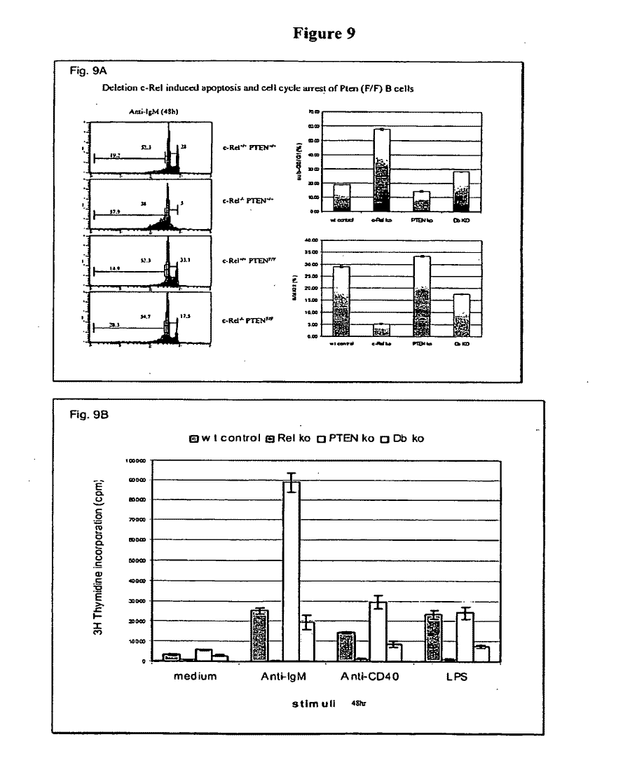

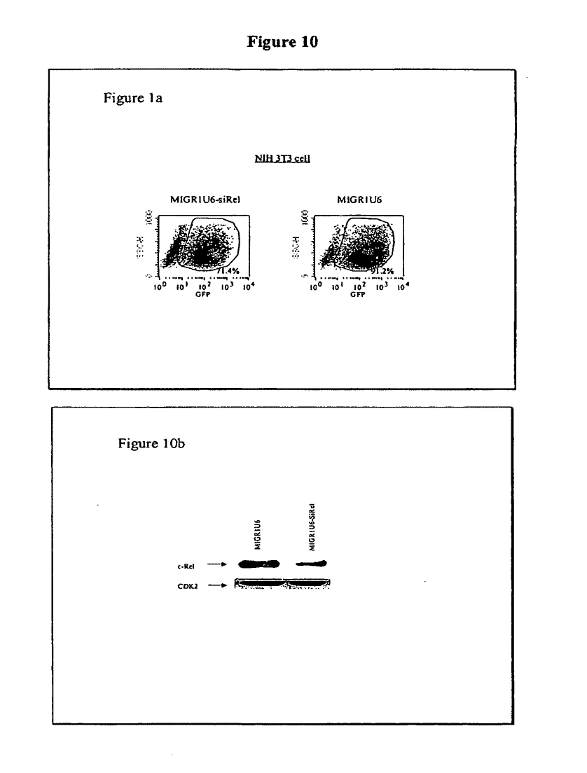

The effect of c-Rel inhibition on Pten-mediated pathological response was tested by breeding Pten-mutant mice with the c-Rel knockout mice. The results indicated that c-Rel deletion suppressed the hyper-proliferation and increased apoptosis of the Pten-mutant B cells, as measured by two different assays (FIG. 9). Taken together, the studies show that blocking c-Rel is sufficient to suppress the hyper-proliferative nature of the Pten-mutant cells and renders the cells prone to apoptosis.

### Example 4

This Example describes the use of siRNA to inhibit c-Rel expression.

### A. Materials and Methods

Construction of siRNA Expressing Vectors

The method used for the construction of siRNA expression vector has been described. Briefly, Mouse U6 promoter was amplified by PCR from mouse genomic DNA using the following oligos: GGAAGATCTATCCGACGCCGCCATCTCTA (SEQ ID NO:4) and sense: antisense: 5′ the GTGGAATTCGTTAAC GAAGACCACAAACAAGGCTTTCTCCAA (SEQ ID NO:5). Within the sense oligo sequence, the Bgl II target sequence is underlined. Within the antisense oligo sequence, the first underlined sequence represents an EcoR I restriction site and the second is a Bbs I site. The PCR product was cloned into the Bgl II and EcoR I sites of pEGFP-C3 vector (Clontech) to generate a new vector, pEGFP-mU6-1.

The siRNA oligos for c-Rel were designed as follows: upper strand: 5′ TTTGGTGTGAAGGGCGATCAGCAGGTTCAAGAGACCTGCTGATCGCCCTTCA CACTTTTTC (SEQ ID NO:6); lower strand: 5′AATTGAAAAAGTGTGAAGGGCGATCAGCAGGTCTCTTGAACCTGCTGATCG CCCTFCACAC (SEQ ID NO:7). Oligos were heated at 95° C. for 5 minutes and then annealed at 37° C. for one hour. The annealed sequence was ligated into the Bbs I and EcoR I sites of pEGFPmU6-1 vector. Then, the siRNA expressing cassette was cut with Bgl II and Hpa I and subcloned into the MIGR1 vector to generate the MIGR1mU6-siRel vector. This vector will co-express siRNA transcript and green fluorescent protein (GFP).

### Preparation of Retrovirus and Determination of Virus Titer

Packaging of retrovirus was performed as described (Houldsworth et al., (2004). Blood. 103, 1862-8). Briefly, the MIGR1mU6-siRel plasmid or the MIGR1mU6 control plasmid was cotransfected with pHIT123 and pCGP into 293T cells using the calcium phosphate method. At 48 hours post transfection, the supernatant was harvested and assayed for viral titer by infection on NIH3T3 cells. The retrovirus supernatant was stored at −80° C. for future use.

### In Vitro Silencing of C-Rel

To test the silencing effect of c-Rel siRNA expressing retrovirus, 2×10⁵ of NIH3T3 cells were seeded in 6-cm dishes. After culture for 24 hours, 100 μl of retrovirus (5×10⁶/ml) was added into 3T3 cells in the presence of polybrene (4 μg/ml). At 48 hours post-infection, cells were harvested and monitored by flow cytometry for the infection efficiency. The expression of c-Rel at the protein level was tested by Western blot using a c-Rel specific polyclonal antibody.

### In Vitro Infection of Wehi-231 Cells

To test the effects of c-Rel silencing on Wehi-231 cell survival and cell cycle progression, 2 ml of the cells (2×10⁶/ml) were seeded in a 6-well plate and cultured with anti-CD40 (10 μg/ml) for 48 hours in the presence of polybrene (4 μg/ml) and different dosages of the c-Rel siRNA expressing retrovirus and the control virus (0.3125, 0.625, 1.25, and 2.5×10⁶). Cells were harvested and analyzed by PI staining for cell survival and cell cycle progression.

### In Vitro Infection of Primary B Cells

To test the effects of c-Rel silencing on B cell response, primary B cells were isolated from mouse spleen and were stimulated for 24 hr with anti-CD40 (10 μg/ml) before addition of the retroviruses. Cells were harvested at 48 hr post-infection and were monitored by propidium iodide (PI) staining analysis for cell survival and cell cycle progression, or by Ki-67 staining for cell proliferation.

Generation of siRNA-Expressing Bone Marrow Chimeric Mice

Generation of the chimeric mice was performed as previously described. Briefly, Donor mice (57 BU6, female, 8-10 weeks old) (The Jackson Labs, USA) were injected with 5-fluorouracil (5-FU, 250 mg (kg weight) per animal. Four days later, bone marrow cells (BMCs) were isolated from tibias and femurs of the mice and were cultured at a concentration of 2×10⁶/ml in 2 ml in a 6-well plate with a cytokine cocktail containing IL3 (6 ng/ml), IL6 (10 ng/ml) and SCF (100 ng/ml). After 24 hours, retrovirus supernatant was added into the BMCs and they were cultured for an additional 4-6 days. Cells were then collected and injected into lethally irradiated mice (850 Rad) through the tail vein. Bone marrow chimeras were analyzed at 4-8 weeks post-bone marrow transfer (BMT). Cell reconstitution in each immune organ was monitored by flow cytometry for the percentage of GFP positive cells.

### Analysis of KLH-Specific Responses

For anti-KLH T cell responses, mice were immunized via hind footpad injection of KLH (100 μg) emulsified with CFA (Calbiochem, La Jolla, Calif.) at a ratio of 1:1. After 9 days, the splenocytes and cells from lymph nodes were separately isolated and were cultured for 60 hr in various concentrations of KLH in RPMI 1640 medium supplemented with 10% FCS. Proliferation was measured by the addition of [³H]thymidine for the last 12 hours.

### Ki-67 Staining

Ki-67 staining was performed as previously described. Briefly, 1×10⁶ cells harvested from cell culture were washed twice with PBS and then fixed for 20 min in 0.5 ml of fixation buffer (eBioscience, San Diego, Calif.). Cells were washed first with PBS and then with permeabilization buffer. Cells were incubated at 4° C. for 10 min in permeabilization buffer and then stained with anti-Ki67 antibody for 30 min. Ki-67 expression cells were quantified by flow cytometry.

### B. Results

### Generation of C-Rel Silencing Retroviral Construct

To construct a c-Rel silencing construct, a 21-nucleotide (nt) sequence unique to c-Rel was inserted downstream of the U6 promoter in the pEGFP-mU6-1 vector. The siRNA-expressing cassette was then sub-cloned into the Bgl II and Hpa I sites of the MIGR1 vector to generate a new vector named MIGRI-mU6-siRel. The presence of an IRES sequence in this vector allows co-expression of c-Rel siRNA duplex and the green fluorescent protein (GFP), the latter being used to monitor transduction efficiency. This vector was co-transfected with two packaging plasmids into 293T cells as described in the Materials and Methods section. Virus was harvested from the cell culture supernatant and gives rise to a titer of 5×10⁶/ml.

### Silencing of C-Rel

To test the silencing effect of the retrovirus on c-Rel expression, NIH3T3 cells were employed due to the constitutive expression of c-Rel in this cell line. The cells were cultured for 24 hr before virus infection. 48 hr post-infection, cells were harvested and monitored by flow cytometry for the infection efficiency reflected by the percentage of GFP+ cells (FIG. 10a). Cell lysates were then prepared and subjected to Western blot analysis for c-Rel protein expression. A reduction in protein expression in the cells infected with c-Rel siRNA expressing virus as compared to the cells infected with control virus was observed (FIG. 10b), indicating the retrovirus is effective in silencing c-Rel expression.

### Silencing C-Rel Results in Diminished Cell Survival and Cell Cycle Progression in Wehi-231 Cells

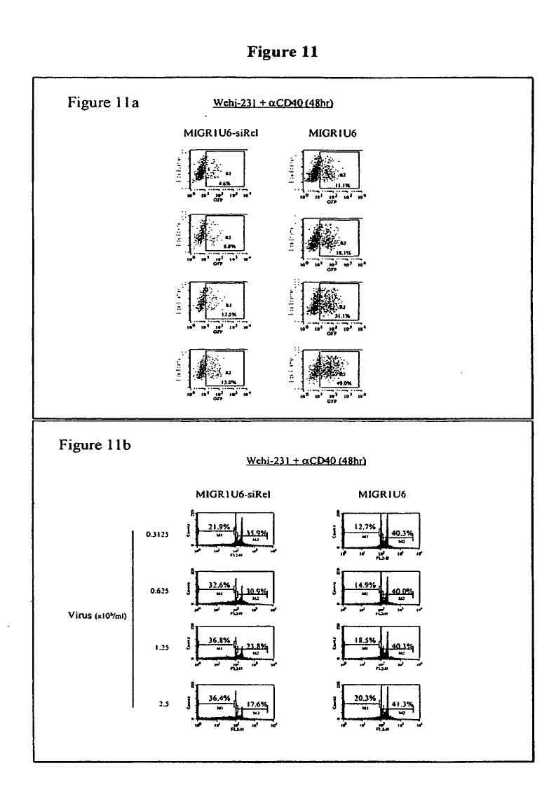

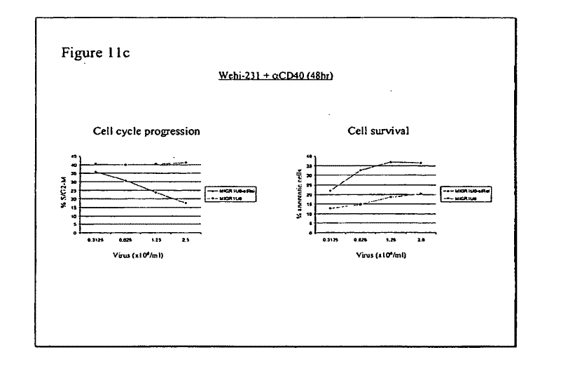

C-Rel has been reported to be over-active in a variety of B cell lymphomas (Rosenwald et al., (2002) N Engl J Med. 346, 1937-47; Houldsworth et al., (2004) Blood 103, 1862-8; Neat et al., (2001) Genes Chromosomes Cancer. 32, 236-43; Barth et al., (2003) Blood. 101, 3681-6). Therefore, suppression of c-Rel activity is useful for the treatment of this type of disease. A B cell lymphoma cell line (Wehi-231) was stimulated with anti-CD40 in the presence of c-Rel siRNA expressing retrovirus or control virus. Cells were harvested at 48 hr post-infection and were analyzed by PI staining analysis for cell survival and cell cycle progression. A dose-dependent reduction in cell survival and cell cycle progression was observed in the c-Rel silencing group (FIG. 11b, 11e). Cells infected with control virus, however, although with a higher infection efficiency, maintained steady state cell cycle progression.

### Silencing C-Rel in Primary B Cells LED to Impaired Cell Survival and Reduced Proliferative Response to Mitogenic or Antigenic Stimulation

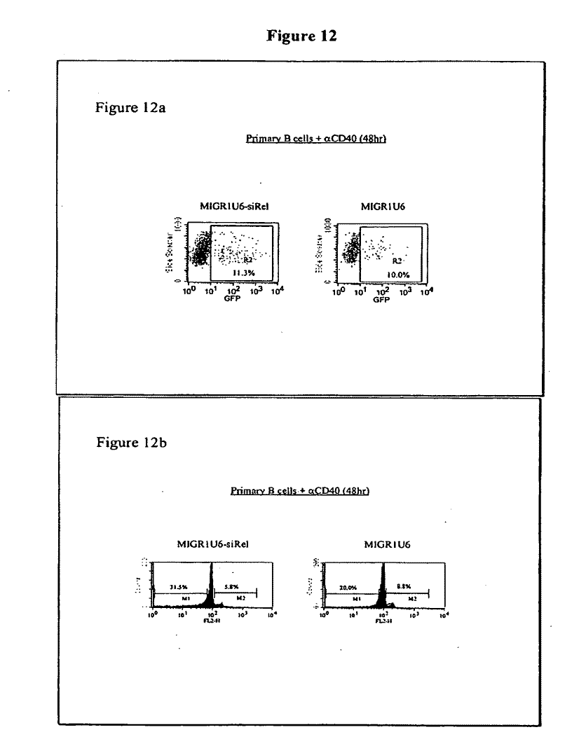

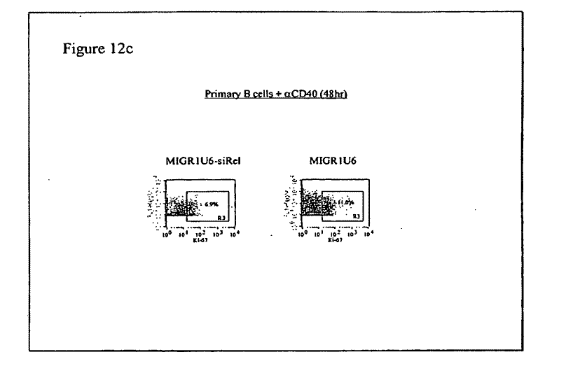

It was previously reported that c-Rel is essential for B cell survival and cell cycle progression (Tumang et al., (1998) Eur J Immunol. 28, 4299-312). Thus silencing c-Rel renders B cells less capable of responding to mitogenic stimulation. To test this, primary B cells isolated from mouse spleen were stimulated for 24 hr with anti-CD40 and then infected with either c-Rel siRNA expressing retrovirus or control virus. As shown in FIG. 12a, up to 11% of the cells could be infected. Infection with c-Rel siRNA expressing virus resulted in increased levels of apoptotic cells and a decreased number of cells in cell cycle progression (FIG. 12b) that could be confirmed by the diminished staining of Ki-67 in the silencing group as compared to the control group (FIG. 12c).

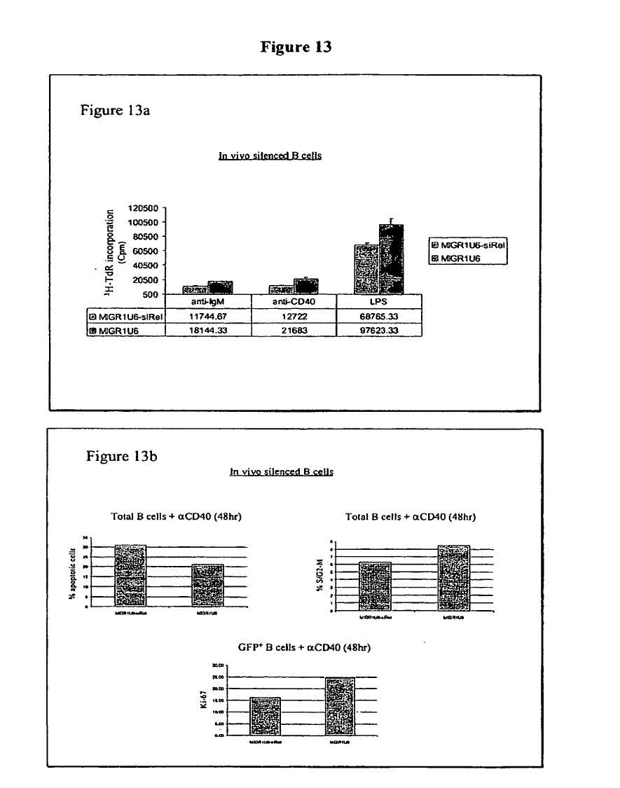

To confirm the observations resulted from the in vitro c-Rel silencing, a c-Rel knockdown chimeric mice (See Materials and Methods) was generated through reconstitution of the virus-infected bone marrow cells in the recipient mice. After two months, some of the chimeric mice were sacrificed and the remaining mice were used for antigen immunization (See below). B cells isolated from the chimeric mice spleen were stimulated for 48 hr with different mitogens including anti-IgM, anti-CD40, and LPS. Cell proliferation was monitored by thymidine incorporation assay. As seen in FIG. 13a, a reduced proliferative response to all of the mitogens was observed in the c-Rel silencing group as compared to the control group. The diminished response to CD40 signaling was further confirmed by PI staining analysis showing more cells undergoing apoptosis and fewer cells entering into cell cycle in the c-Rel silencing group, and also confirmed by Ki-67 staining which was significantly reduced upon c-Rel silencing (FIG. 13b).

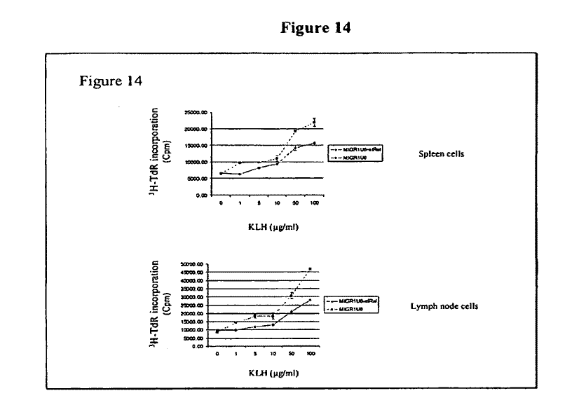

To test the consequence of c-Rel silencing in a more physiological condition, the chimeric mice were immunized through the hind footpad with KLH (100.0 μg). Nine days later, lymphocytes were isolated separately from spleen and lymph node and were cultured with KLH at different concentrations. After 48 hr, cell proliferation was monitored by thymidine incorporation. Results showed that lymphocytes from either immune organ (spleen or lymph node) in the c-Rel silencing group exhibited a significantly diminished proliferative capacity as compared to that in the control group (FIG. 14).

### Example 5

This Example describes additional oligos used for c-Rel RNAi

A. Synthesized Oligos:

| | |
|---|---|
| Oligo 1: | |
| Sense: | |
| 5′ AATGTGAAGGGCGATCAGC 3′ | (SEQ ID NO: 8) |
| Antisense: | |
| 5′ GCTGATCGCCCTTCACATT 3′ | (SEQ ID NO: 9) |
| siRNA seq: | |
| 5′ GCUGAUCGCCCUUCACAUU 3′ | (SEQ ID NO: 10) |
| Oligo 2: | |
| Sense: | |
| 5′ AATGTGAAGGGCGATCAGCAGGT 3′ | (SEQ ID NO: 11) |
| Antisense: | |
| 5′ ACCTGCTGATCGCCCTTCACATT 3′ | (SEQ ID NO: 12) |
| siRNA seq: | |
| 5′ ACCUGCUGAUCGCCCUUCACAUU 3′ | (SEQ ID NO: 13) |

B. Vector-Expressed Oligos:

| | |
|---|---|
| 5′ GTGTGAAGGGCGATCAGCAGG 3′ | (SEQ ID NO: 14) |
| Sense | |
| 5′-CCGUGCUCCAAAUACUGCA-3′ | (SEQ ID NO: 15) |
| Antisense | |
| 5′-UGCAGUAUUUGGAGCACGG-3′ | (SEQ ID NO: 16) |

More siRNA sequences for c-Rel gene

C. Oligos for Human C-Rel RNAi

si-Rel 1 (20 nt) (position 256 to 275)
(SEQ ID NO: 17)
5′ TGT GAA GGG CGA TCA GCA GG 3′
si-Rel 2 (23 nt) (position 309 to 330)
(SEQ ID NO: 18)
5′ CCG AAC ATA CCC TTC TAT CCA GA 3′
si-Rel 3 (22 nt) (position 424 to 445)
(SEQ ID NO: 19)
5′ GAC TGC AGA GAC GGC TAC TAT G 3′
si-Rel 4 (23 nt) (position 549 to 573)
(SEQ ID NO: 20)
5′ GGC AGG AAT CAA TCC ATT CAA TG 3′
si-Rel 5 (23 nt) (position 601 to 623)
(SEQ ID NO: 21)
5′ GAT TGT GAC CTC AAT GTG GTG AG 3′
si-Rel 6 (22 nt) (position 655 to 676)
(SEQ ID NO: 22)
5′ CAT GGT AAT TTG ACG ACT GCT C 3′
si-Rel 7 (22 nt) (position 877 to 898)
(SEQ ID NO: 23)
5′ GCT GAT GTA CAC CGT CAA GTA G 3′
si-Rel 8 (23 nt) (position 1327 to 1349)
(SEQ ID NO: 24)
5′ GCA GAA TCC TAC TAT CCC TCA CC 3′
si-Rel 9 (21 nt) (position 1769 to 1789)
(SEQ ID NO: 25)
5′ GAG ACT TGA GAC AGC TCC ATC 3′
si-Rel 10 (22 nt) (position 1957 to 1978)
(SEQ ID NO: 26)
5′ GAT AGT CAG TAT TCA GGT ATT G 3′

D. Oligos for Mouse C-Rel RNAi

si-Rel 1 (21 nt) (position 478 to 498)
(SEQ ID NO: 27)
5′ GTG TGA AGG GCG ATC AGC AGG 3′
si-Rel 2 (20 nt) (position 619 to 638)
(SEQ ID NO: 28)
5′ GCC TCA TCC TCA TGA TTT AG 3′
si-Rel 3 (22 nt) (position 671 to 693)
(SEQ ID NO: 29)
5′ GCA GAA TTT GGA CCA GAA CGC AG 3′
si-Rel 4 (21 nt) (position 765 to 785)
(SEQ ID NO: 30)
5′ GGA TTA GTG CAG GAA TCA ATC 3′
si-Rel 5 (21 nt) (position 824 to 844)
(SEQ ID NO: 31)
5′ GAC TGC GAC CTC AAT GTG GTG 3′
si-Rel 6 (22 nt) (position 878 to 899)
(SEQ ID NO: 32)
5′ GAT GGT AAC TTC ACA ACT GCT G 3′
si-Rel 7 (20 nt) (position 960 to 979)
(SEQ ID NO: 33)
5′ GGA TCT TAG CCC GTG TGA AC 3′
si-Rel 8 (22 nt) (position 1103 to 1124)
(SEQ ID NO: 34)
5′ GCT GAT GTA CAC CGC CAA GTA G 3′
si-Rel 9 (22 nt) (position 1157 to 1178)
(SEQ ID NO: 35)
5′ GCT ATA CTG GAG CCT GTG ACA G 3′
si-Rel 10 (23 nt) (position 1463 to 1485)
(SEQ ID NO: 36)
5′ GCT GAA CCT TAC TAT TCT TCA TG 3′

### Example 6

This Example describes additional small molecule inhibitors of c-Rel.

The FP assay described in Example 1 was used to perform a large high-throughput screen to identify small molecules that inhibit the interaction between c-Rel protein and the targeted KB sites CD28RE. This screen involved 16,000 compounds and generated about 100 bits that reduced FP signals by more than 45% (provide concentrations used of the screened compounds). The hits were then independently tested in the EMSA assay. After the secondary screen, 19 compounds were determined to have significant inhibitory activity in disrupting formation of c-Rel DNA binding complexes. The molecules that showed significant inhibitory activity in the secondary screen fall into two distinct categories, designated Class I and Class II (FIG. 15). All incorporate aryl groups, indicating that they have some rigidity and hydrophobic character. Class I compounds (IT-101 to IT-113) have no net charge. These molecules all include a barbituric acid derived moiety in their structures. Class II molecules are derivatives of naphthalene, quinolinones, and chromenone.

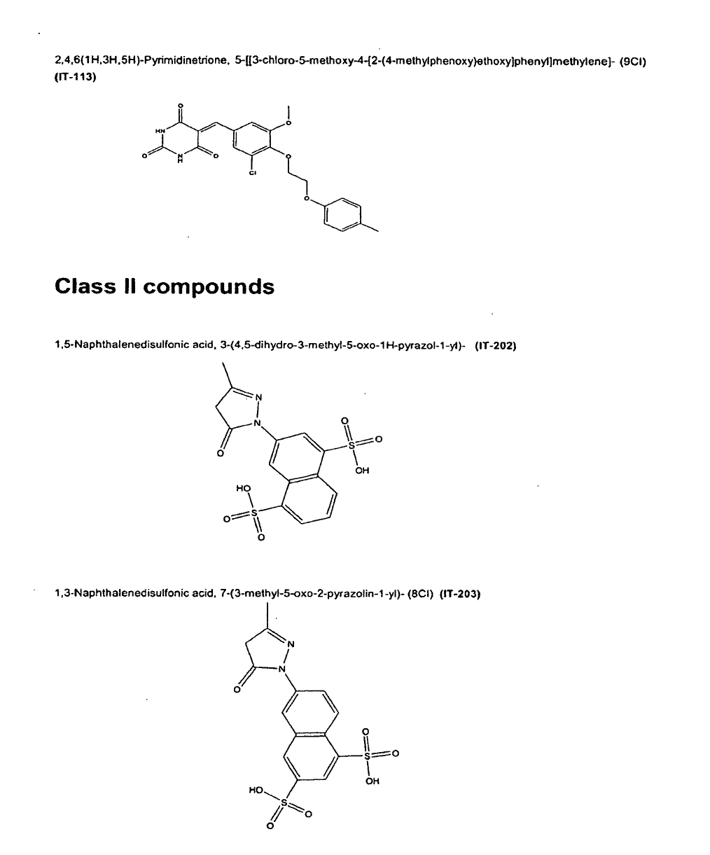

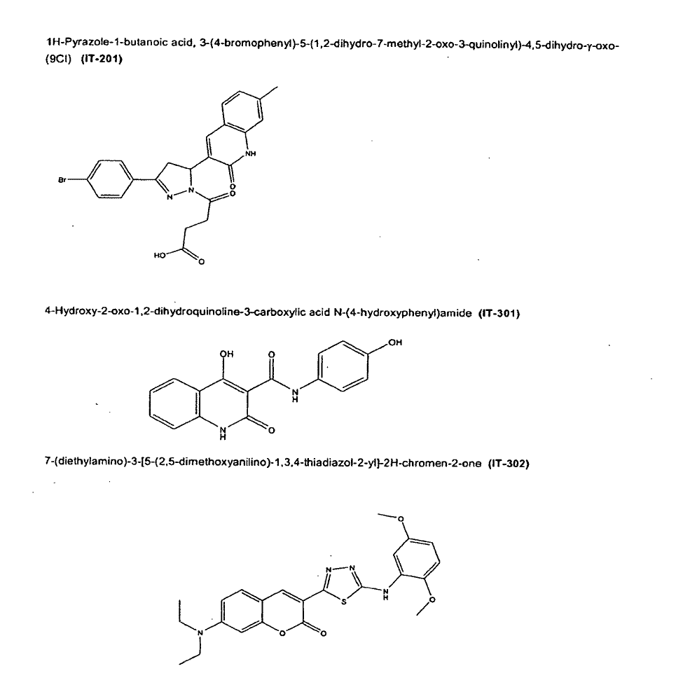

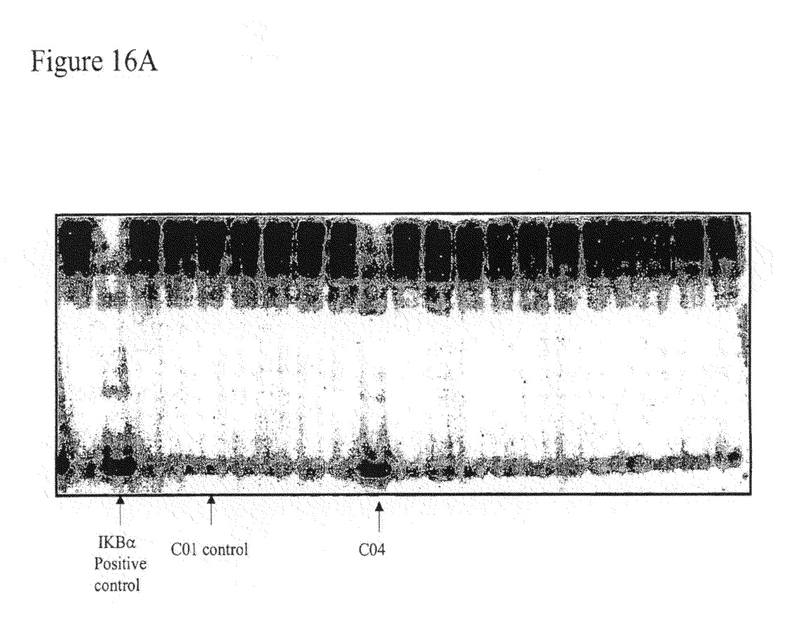

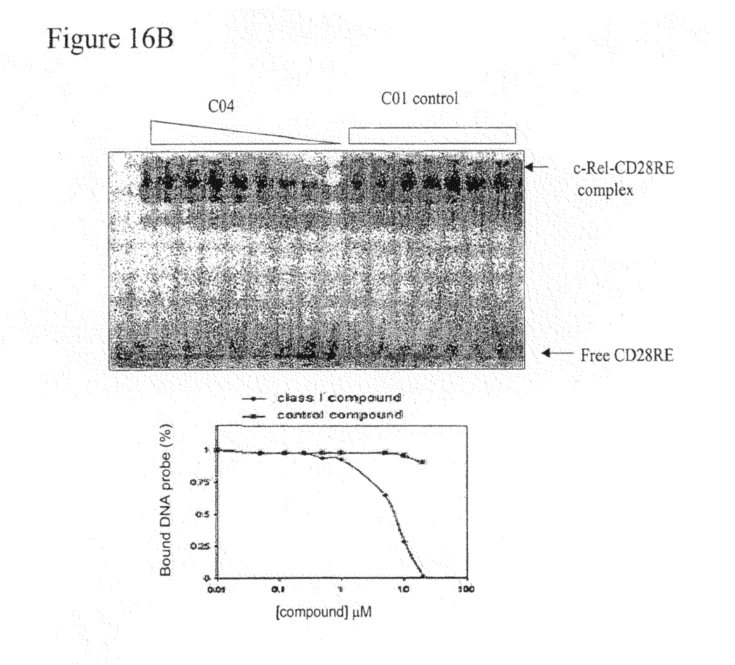

FIG. 16A shows an example of using EMSA to validate the DNA inhibitory activity of hit compounds. The inhibitory potency of positive compounds was further quantitated by IC50 measurement. As shown in FIG. 16B, one of the Class I compounds (black solid circle line) has a IC50 value of 6 μM and the control compound (purple solid circle line), has little inhibitory effect at all in the condition tested, indicating that Class I compounds are suitable for further optimization.

### Example 7

This Example describes methods for further analysis and optimization of lead compounds.

### Measure Compound Bioavailability

To better understand the biological availability of the compounds members of this class are synthesized with radiochemical labels and with stable isotope labels to assess cell penetration and metabolism within cells (see below). Radioisotope labels can determine uptake of the inhibitors within cells but cannot allow determination of metabolic changes of the compound. Correspondingly stable isotope labeled compounds are used to precisely quantitate the amount of drug that is in unmodified form in cells to establish a cellular concentration. In conjunction, these methods are employed to establish both drug uptake and the fraction converted by metabolism to altered forms.

2-¹³C or ¹⁴C labeled versions of Class I derivatives are synthesized for cell penetration studies. It is contemplated that as little as 100 pCi (200 cpm) can be detected by radioactive scintillation counting (a specific activity 60 mCi/mmol corresponds to a 2 pmol detection limit). 50 mg of cells obtained from a 15 cm round culture plate are assayed, and a concentration down to 40 nM within a cell is determined. This sensitivity is expected to measure a level of drug required to achieve biological effect in cells (500 nM-50 μM). Independently ¹³C labeled compound is added to drug treated cells as an internal standard (typically 100-1000 pmol) and after homogenization and fractionation mass spectroscopy (ESI and MALDI) is used to determine the ratio of labeled to unlabeled compound in the sample. The determined mass ratio of labeled/unlabeled ions precisely quantifies the drug amount in the sample. Other work has measured concentrations down to 50 nM of nicotinamide with as little as 50 μL of a biological sample (Sauve A. et. al 2005, Mol Cell 17:595). In addition, a variety of isotope labeled molecules including ¹⁸O-NAD, ¹⁸O-nicotinamide (Suave et al., supra), and ¹⁸O-nicotinamide riboside are available for determining the levels of these metabolites in cultured cells and in biological tissues. Typical sensitivities are in the pmol to fmol range. Both Caco2 and lymphoid cell lines are used for bioavailability experiments.

### Inhibition of IL-2 Production in T Cells

The hallmark of c-Rel function is in the regulation of cytokine gene expression. In T cells, many studies have demonstrated that c-Rel plays a critical role in IL-2 gene expression in T cells (Liou et al., 1999, Int Immunol 11:361; Kontgen et al., 1995, Genes & Dev. 9:1965). IL-2 promoter contains multiple transcription factor binding sites, among which the CD28 RE and the NF-AT/Ap1 composite site are functionally shown to be involved in IL-2 gene expression (Chen and Rothenberg, 1993 Mol Cell Biol 13:228; Jain et al., 1992 Nature 356:801; Rooney et al., 1994 Embo J 13:625; Jain et al., 1993 J Immunol 151:837; Ghosh et al., 1993 Proc. Natl. Acad. Sci. USA 90:1696; Harhaj et al., 1996 Mol Cell Biol 16:6736). Interruption of c-Rel, NF-AT, or AP-1 activation by pharmacological inhibitors or with specific gene knockout led to impaired IL-2 production (Jain et al., 1992, supra; Jain et al., 1993, supra; Rooney et al., supra). IL-2 expression in T cells is used to validate the intracellular efficacy of the c-Rel inhibitory compounds. IL-2 expression is measured by Real-Time PCR, intracellular staining with IL-2 specific antibody and analysis by flow cytometry, and ELISA as described (Cheng et al., 2003 Oncogene 22:8472; Boffa et al., 2003 Cell Immunol 222:105; Tian et al., 2005

Cell Immunol 234:39; Tian et al., 2005 Cell Immunol 235:72). T cell lines such as Jurkat, D5h3 CD4+ T cells are used for initial testing.

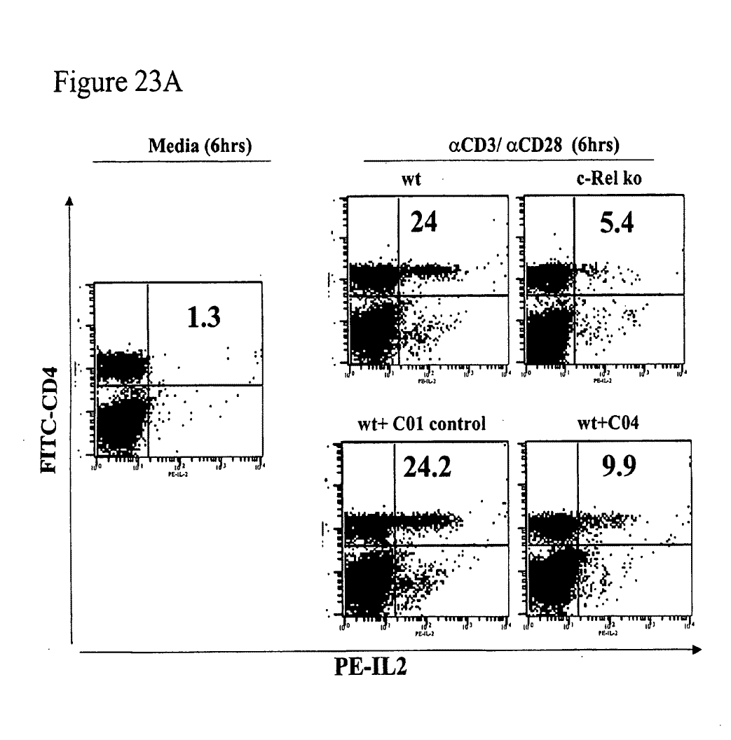

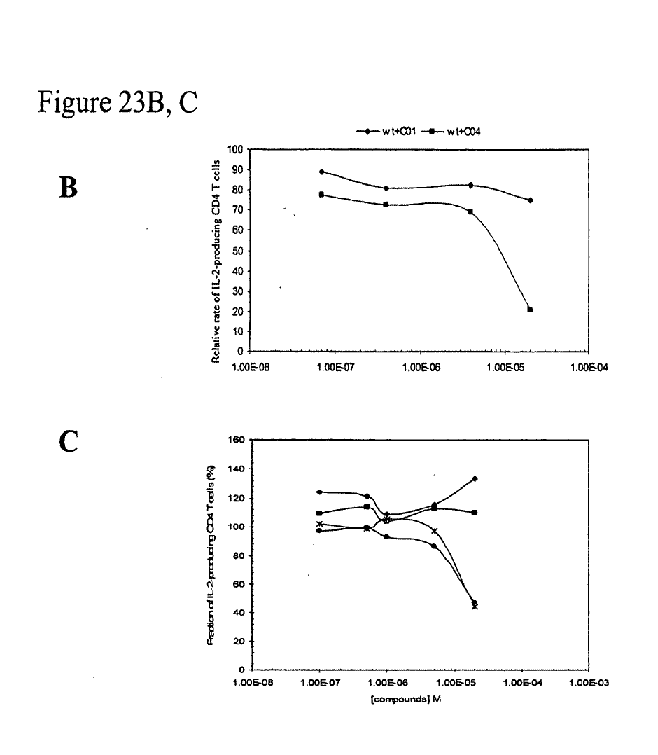

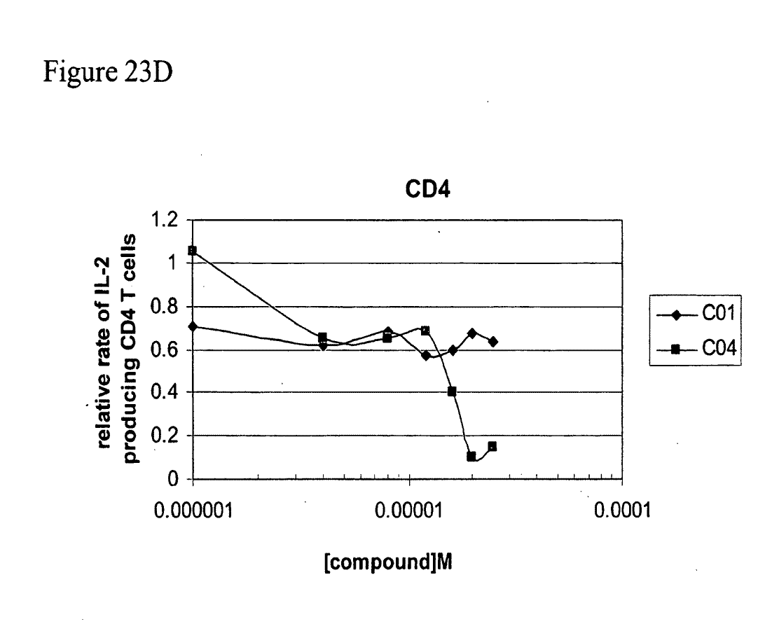

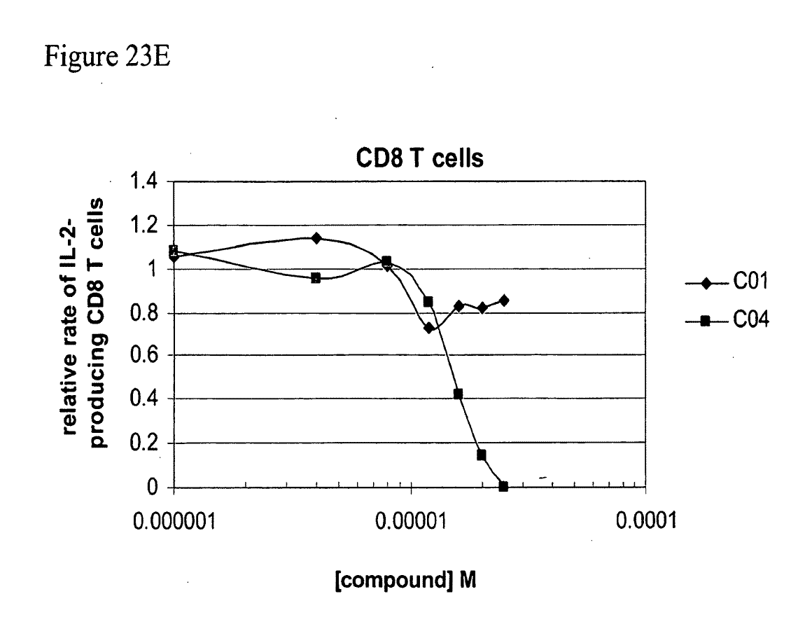

Primary T cells are utilized. FIG. 23A is an example of measuring the effect of c-Rel compounds on IL-2 expression using intracellular IL-2 staining. C-Rel KO CD4+ T cells were utilized as a positive control, which demonstrated a diminished IL-2-expression as compared to the c-Rel wild type T cells. One of the c-Rel compound (C04) also partially diminished IL-2 expression, whereas the control compound (C01) has no inhibitory effect on IL-2 production in CD4+ T cells.

Taken together, C04 demonstrated IL-2 and IFN-γ inhibitory activity at IC50˜9 uM, whereas C01 has poor activity (FIG. 23B, D, E). In addition, two other compounds demonstrated IL-2 and IFN-γ inhibitory activity with IC50s in the 5-10 uM range (FIG. 23C). Furthermore, these compounds do not have an inhibitory effect on non-c-Rel target genes (e.g. CD4, CD8).

### Inhibition of Cytokine Production in T Cells

The IT compounds and derivatives that demonstrate reasonable IC50 (e.g. <1 uM) in the biochemical assay (EMSA) are tested for efficacy in blocking IL-2 expression at the cellular level. C-Rel plays a critical role in IL-2 gene expression in T cells and IL-2 has been a therapeutic target of several existing immunosuppressive medicines. Thus, IL-2 expression in T cells is used to validate the intracellular efficacy of the c-Rel inhibitory compounds. IL-2 mRNA level stimulated with an anti-CD3 signal for various time-points is measured by Real-Time PCR. IL-2 protein level are measured by intracellular staining with IL-2 specific antibody and analyzed by flow cytometry as described previously. Cell lines such as Jurkat, D5h3 CD4+ T cells are used for initial testing. Subsequently, primary T cells are utilized.

The compounds that exhibit inhibitory effects on IL-2 expression are further tested to see if they also block the expression of other c-Rel target genes including IFN-γ, IL-12, TNF-α in various immune cells. Additionally, the effect of IT compounds on the expression other c-Rel downstream target genes listed herein is used as a basis of assessment.

As negative controls, genes (e.g. GAPDH, CDK2) that are not regulated by NF-kB or c-Rel are also examined to ensure that the inhibitory effect is specific. Yet another good control is cells derived from the c-Rel knockout mice. It is anticipated that c-Rel inhibitory compounds modulate gene expression profile and cellular response that are identical or similar to that of the c-Rel knockout mice.

### Inhibition of B Cell Proliferation and Survival

C-Rel plays an important role in regulating B cell proliferation and survival. Deletion of c-Rel gene in mouse led to apoptosis and cell cycle block in mature B cells in response to the B cell antigen receptor-signaling (Owyang et al., 2001 J Immunol 167:4948; Cheng et al., 2003 Oncogene 22:8472; Hsia et al., 2002, Int Immunol 14:905). The specific target genes of c-Rel in B cells include anti-apoptotic molecules such as Bcl-x and cell cycle regulators such as E2F3a (Cheng et al., 2003, supra; Hsai et al., 2002, supra). C-Rel has also shown to confer viability for many B cell tumor cell lines. Thus, these biological activities of c-Rel can be used to assess intracellular efficacy of the c-Rel inhibitory compounds. Cell apoptosis and cell cycle progression are measured by propidium iodine staining and flow cytometry as described previously (Liou, 2001, supra; Cheng et al., 2003, supra; Hsai et al., 2002, supra). B cell lines such as WEHI231, Ramos (or Raji) are used for initial testing. Subsequently, primary B cells are utilized.

### Synthetic Approaches for Class I Compounds

Synthetic strategies to obtain diversity in Class I (FIG. 16) are attractive for both brevity and for achievement of rapid structural diversity for SAR studies. Members of this class are synthesized by coupling barbituric acid to aldehydes under conditions that promote condensation. This chemistry is very old and prototypical examples date to at least 1900, e.g. a report of condensation of benzaldehyde with barbituric acid. These condensations occur rapidly in water or alcohols and are catalyzed by acid or basic conditions.

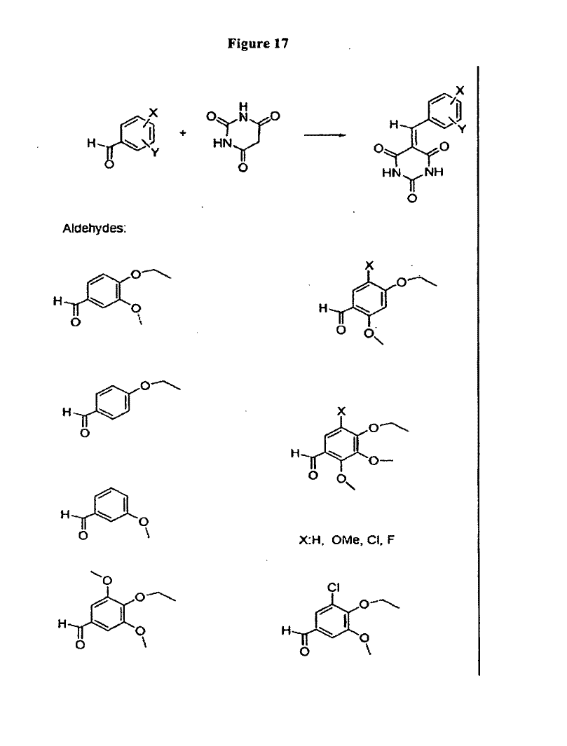

The syntheses are expected to yield pseudosymmetrical or non-symmetrical compounds. In other words, compounds will contain either one or two barbituric acid moieties as determined from the nature of compounds I-III (FIG. 16). In the non-symmetrical class a number of very simple compounds are readily obtained related to the parent 3-methoxy-4-alkoxy-5-benzylidene-pyrimidine-2,4,6-trione conjugates of compounds (I) and (III). Commercially available 3-methoxy benzaldehyde, 3-methoxy-4 ethoxybenzaldehyde and 4-ethoxyaldehydes are condensed with barbituric acid to form the related barbituric acid benzylidene conjugate (FIG. 17). In addition, trisubstituted compounds containing 3, 4, 5 substitution patterns are readily constructed. It is contemplated that the 3 methoxy-4 alkoxy group is important for molecular interaction of compounds I and III.

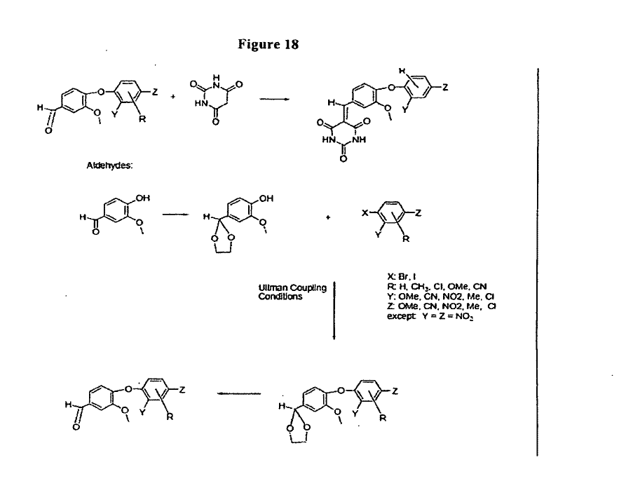

Within this compound class, additional modifications include increasing substitution on the aryl ring and exploring movement of the alkoxy positions to other ring positions. Furthermore, substitution of the alkoxy groups is examined to determine if these groups can enhance either binding or biological activity. Immediate access to these structures is permitted by synthetic simplicity and the commercial availability of a variety of aryl aldehydes. These compounds are initially synthesized on a 200 mg scale. Variations in compounds within a diaryl series as exemplified by compound I in Class I are also synthesized. This diaryl series is accessed via employment of Ullman coupling procedures (FIG. 18).

In some embodiments, the phenolic aldehyde is protected and commercially available aryl bromide and iodides are then coupled with Ullman conditions in which Cu salt and a Cu ligand is added to the aryl components as a coupling catalyst (Cristau et al., 2004 Org Lett 6:913). These synthetic transformations are anticipated to produce a diverse set of diaryl ether structures that incorporate different combinations or functional groups analogous to those found in compound I. In addition, aryl substitution is changed or increased on the second aryl ring to identify optimal c-Rel binders.

### Class I Symmetrical Derivatives

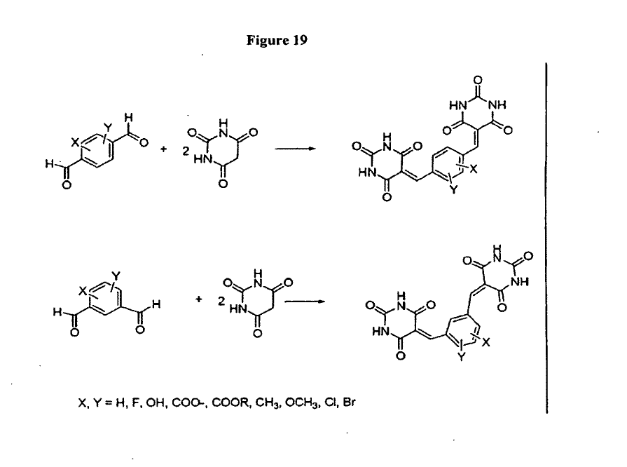

Synthesis of pseudo-symmetrical derivatives analogous to compound II is performed from the respective dialdehyde building blocks (Placer et al., 1966 Anal Biochem 16:359) to evaluate if these compounds function as c-Rel inhibitors. Terephthalaldehyde and isophthalaldehyde derivatives bearing substitution are synthesized (FIG. 19). These compounds are commercially available with a number of modifications that allow for the evaluation of structure activity relationships within this family of compounds. These derivatives include F, OH, OMe, and COOH modifications at the other aryl positions.

### Synthesis of Radiolabeled or Isotopically Labeled Compounds for Evaluation of Bioavailability

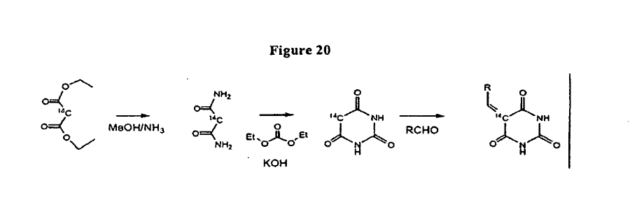

To determine biological availability of compounds in Class I, a general and rapid synthetic scheme to incorporate radiochemical or stable isotope labels is desired. A straightforward synthesis of barbituric acid exists in the literature in which malonamide is reacted with diethylcarbonate to yield barbituric acid in excess of 55% yield (Shimo et al., 1959 J. Org. Chem. 24:19). ¹⁴C or ¹³C are incorporated by this method (FIG. 20) after formation of malonamide from the commercially available ¹⁴C or ¹³C labeled diethylmalonates (2-¹⁴C available at 60 mCi/mmol, 2-13C 99% incorporation). Incorporation of the labeled barbituric acid into any desired Class I derivative is straightforward via condensation with the appropriate aldehyde. The complete procedure for this methodology is shown in FIG. 20.

### Class II Synthesis

In another approach, Class II compounds are modified to determine the need for carboxylic acid moieties and if changes in the molecular ingredients of these molecules at their carboxylate positions can alter antagonism of c-Rel in molecules presenting modifications at these moieties. The approach includes a synthetic sequence of esterification, reduction or alkylation to evaluate ketone, alcohol and uncharged carboxylate functionalities for binding.

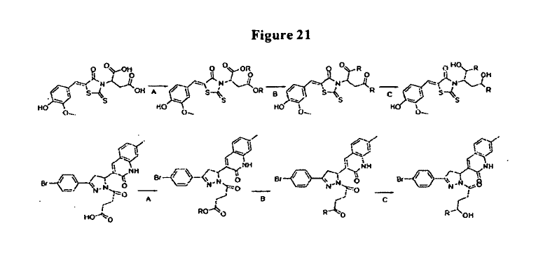

Charge requirements in class II compounds are evaluated by varying charge systematically in both subclasses of compounds (carboxylates and sulfonates). As shown in FIG. 21, modest modifications in the carboxylate moieties of these molecules are synthetically accessible and can be instructive. In this series, a synthetic sequence that includes esterification, ketone synthesis and reduction is utilized. Each of the intermediates thereby generated is looked at for biochemical or biological activity. For example, it is determined if esterification abrogates biochemical activity but enhances cell permeability. If so, it is concluded that esterification represents a bioavailable pro-drug form of the molecule that can penetrate the cell and be hydrolyzed back to the carboxylate upon action of cell esterases.

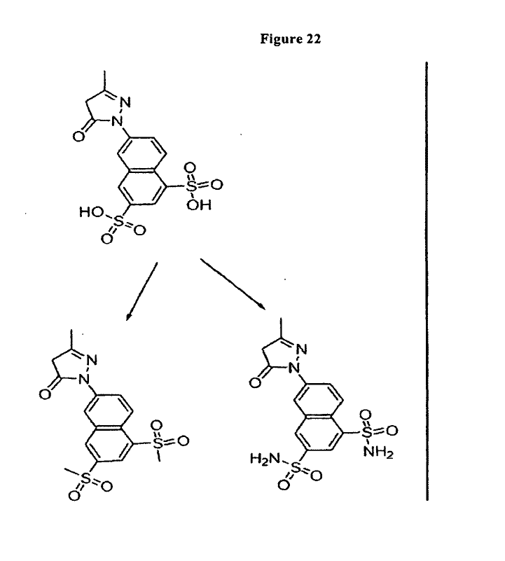

Sulfonic acids are not expected to penetrate cells with any facility. In some embodiments, modifications that convert the inhibitors to neutral sulfone or sulfonamide groups are utilized (FIG. 22). Compounds are treated with conditions (PCl5) that yield the sulfonyl chloride and then treated with a methyl Grinard or ammonia to achieve the sulfone or amide synthesis respectively. These molecules are expected to be uncharged and may penetrate cells. These molecules are evaluated in biochemical and biological assays for activity.

### SAR Studies of C-Rel Inhibitor Analogue Series

SAR studies are performed on the synthetic derivates described above using the biochemical and cellular assays described above to assess their biochemical and cellular inhibitory potency, selectivity, and bioavailability. The initial analyses will allows for the identification of functional groups or structural components that are crucial for activity as well as those that are available for manipulation to enhance other properties such as permeability.

Computational models are used to incorporate empirical SAR data and assist further SAR modification and improve functional properties. Lead series that fulfill certain criteria as best drug candidate with property profiles suitable for further preclinical and clinical development are identified. The set criteria including activity, selectivity and pertinent physicochemical properties, plus an initial evaluation of ADME and certain safety attributes using computational tools.

In some embodiments, computational tools described in FIG. 12 are utilized. Additional programs are used to predict bioavailability and potential metabolic liability or toxicity profile (e.g., LD1.0, TOPKAT (Enslein et al., 1994 Mutat Res 305:47; Zheng et al., 2005 J Chem Inf Model 45:856), BP neural networks for estimating carcinogenicity, ZGENTOX for predicting mutagenic probability and programs for calculating molecular solubility and hydrophobicity). Docking programs are used to assess potential binding mode of the ligands to target (e.g., GAsDock (Li et al., 2004 Bioorg Med Chem Lett 14:4671)).

### Selection Criteria for Analogues Series in Biochemical and Cellular Assays

An estimated ˜30 Class I and ˜20 Class II compounds are analyzed for IC50 against c-Rel-CD28RE interaction using EMSA as described above. Next, these analogues are tested with selectivity and affinity assays to identify analogues that exhibit specific inhibitory activity toward c-Rel but not E2F1, as well as those that exhibit at least 10× higher selectivity and affinity toward c-Rel than p65 or p50. The threshold or criteria for biochemical potency and selectivity measured by EMSA are:

(c-Rel) EC50<1 uM
(c-Rel) EC50<1/10 of (p50 or p65 or E2F1) EC50
(c-Rel) K_{D}<1/10 of (p50 or p65 or E2F1) K_{D}

Compounds that meet the above criteria are further advanced for bioavailability and cellular potency as demonstrated by the ability to inhibit at least 50% of the control levels of IL-2 production, B cell proliferation, and survival. Analogues that meet both biochemical and cellular potency and selectivity are used as lead compounds for future preclinical studies.

### Example 8

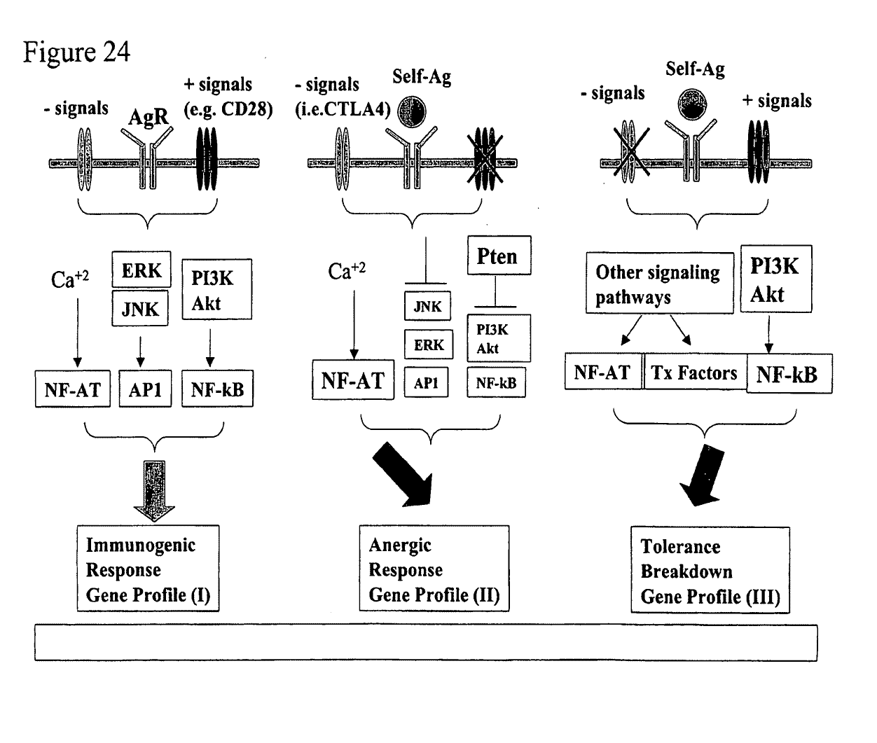

This example describes the PI3K-c-Rel/NF-kB pathway as a signaling integration point for determining immunogenicity, immune tolerance, and autoimmunity (FIG. 24).

### A. Experimental Procedures

Generation of Mouse Strains

Wild type mice, c-Rel−/− mice, c-Rel+/+ Bcl-xL Tg mice, and c-Rel−/−Bcl-xLTg mice (all on C57BU6 background) were generated as described previously (Owyang et al., J Immunol 167:4948). c-Rel−/− JNK2−/−, c-Rel−/− E2F1−/−, c-Rel−/− p53−/−, c-Rel−/− CD19cre/+PTENflox/flox mice were generated by interbreeding c-Rel−/−mice with JNK2−/− mice, E2F1−/−mice, p53−/− mice, CD19cre/+PTENflox/flox mice, respectively, and subsequent crossbreeding to obtain double knockouts. JNK2−/−mice, E2FI−/−mice, p53−/− mice were purchased from Jackson Laboratories. To generate B cell-specific PTEN-deficient mice, PTENflox/+ mice were crossed with CD19Cre/+ transgenic mice, also purchased from Jackson Laboratories. Mouse genotyping was performed by PCR using tail DNA as described previously. Mice 8-12 weeks old were used in all experiments. The mice were maintained under specific pathogen-free conditions at Weill Medical College of Cornell University.

Cell Culture

Mature and immature B cells (>95% B220+) were isolated from untreated mice or sublethally irradiated mice by complement-mediated lysis and Percoll gradient as described previously, and cultured in RPMI 1640 media containing 10% fetal calf serum (FCS) (Cellgro), 1% penicillin, 1% streptomycin (both Life Sciences BRL), and 50 uM b-mercaptoethanol (Sigma). For caspase experiments, 2×106/ml B cells were plated per well in 24-well polystyrene flat-bottom plates (Corning) in 1000 ul of total culture volume. The following combinations of agents were added for 12 hr or 24 hr at 37° C.: 10 mg/ml of goat anti-mouse IgM F(ab)₂ (anti-IgM, Jackson ImmunoResearch Laboratories), 50 mM of z-VAD-fmk, a pan-caspase inhibitor, or 50 mM of z-LEHD-fmk, a specific caspase 8 inhibitor, or 50 mM of z-FA-fmk, a cathepsin B inhibitor (Enzyme System Products). For the PI3K assay, 5×106 B cells were plated per well in 24-well polystyrene flat-bottom plates (Corning) in 2 ml final culture volume, with 10 mg/ml of goat anti-mouse IgM F(ab′)₂ added for the indicated time at 37° C. Following culture, B cells were immediately put on ice, washed 1× with cold PBS, then lysed by the buffer shown below.

Flow Cytometry

The following antibodies were used for FACS analysis: anti-CD24 (HSA), anti-CD21, anti-IgD, anti-IgM, and anti-B220 (RA3-6B2), labeled with R-phycoerythrin (PE), FITC or Allophyocyanin (APC), all purchased from Pharmigen. Isotype-matched immunoglobulin was used as a non-specific staining control for all staining experiments. Cells were stained with surface markers for 30-60 min at 4° C., then washed in 1×PBS and resuspended in 0.5 ml PBS with 2% FCS. For propidium iodide staining assays, 2×106/ml of B cells were cultured in 24-well flat-bottom plates with 10 mg/ml of goat anti-mouse IgM F(ab′)2. At the indicated time points, cells were collected and stained with a solution containing 50 mg/ml propidium iodide, 20 ng/ml RNase A, 0.1% Triton X-100, and 0.1% sodium citrate. Duplicate samples were then analyzed by FACS (Becton-Dickinson) using CellQuest software, and the percentage of apoptotic cells (<2N DNA content) or S and G2/M phase cells (>2N DNA content) quantified. For PIP3 assays, PIP3 levels were measured using a biotin-labeled antibody against anti-PI-3,4,5-P3 (a gift from Dr. Paul Neilsen, Echelon, Salt Lake City, Utah) and FACS following a published protocol.

Measurement of Mitochondrial Membrane Potential

Cells were cultured at a density of 0.5−1.0×106 cells/ml. For each condition, 1 ml of cells were treated for the indicated time. Mitochondrial membrane potential was assayed using the fluorescent potentiometric dye JC-1 (5,5′,6,6′,-tetrachloro-1,1′,3,3′-tetraethylbenzimidazolylcarbocyanine iodide, from Molecular Probe). JC-1 is a cationic carbocyanine dye that accumulates in the mitochondrial space in a membrane potential-dependent manner. The dye exists as a monomer at low concentrations and yields green fluorescence, similar to fluorescein. At higher concentrations, the dye aggregates in a manner that leads to a broad excitation spectrum and an emission maximum at ˜590 nm, similar to phycoerythritin (PE). Briefly, 0.3 ml of the cells was mixed with 0.3 ml of staining solution (complete medium containing 0.5 μg/ml JC-1). Cells were stained for 30 min in a 37° C. incubator (5% CO2). After staining, cells were washed at room temperature in 1×PBS. The cell pellet was then resuspended in PBS, and JC-1 fluorescence analyzed by FACS.

Immunoprecipitation and PI3K Activity Assays

The Protocol was modified from a previously published version. Cells were lysed at 4° C. in 200 μL ice-cold lysis buffer (137 mM NaCl, 2.7 mM KCl, 1 mM MgCl2, 1 mM CaCl2, 1% NP40, 10% glycerol, 1 mg/mL BSA, 20 mM Tris, 0.5 mM sodium orthovanadate, 0.2 mM PMSF, 10 μg ml-1 leupeptin, pepstatin A and aprotinin). Agarose beads were prepared by adding 2 μL of anti-phosphotyrosine (1 μg/μL clone 4G10, Upstate Biotechnology, catalog# 16-125), and 50 μL (25 μL packed beads) of PBS-washed protein A agarose bead slurry to 500 ul PBS in a microcentrifuge tube. The reaction mixture was gently rocked at 4° C. for 1 hour, centrifuged by gentle pulsing to precipitate beads, supernatant discarded, washed 2× times with cold PBS, and resuspended in an appropriate volume of PBS. Immunoprecipitation was carried by incubating 50-80 μg of cell lysate (diluted to 450 μL with PBS) with 50 ul of agarose beads for 1 hour at 4° C. with gentle rocking. Beads were then washed 2× with cold PBS, 1× with kinase buffer (10 mM Tris, 10 mM MgCl2). The kinase reaction was carried out by resuspending pelleted beads in 50 μL kinase buffer containing 10 μg of phosphatidylinositol (PI), 10 uCi g-32P-ATP per 50 ul reaction, and incubated for 30 min at room temperature. Lipids, including PI3K-catalyzed synthesis product PIP, were then extracted from the reacting mixture with 150 μL chloroform. The amount of PI phosphorylated by PI3K was analyzed by thin layer chromatography (TLC) using CH3CI: CH₃OH:H2O=90:70:14.6 as a developing agent.

Immunoblot Assays

Cells were lysed in 50 mM HEPES (pH 7.5), 150 mM NaCl, 1 mM EDTA, 2.5 mM EGTA, 1 mM DTT, 0.1% Tween-20, 10% glycerol, 10 mM b-glycerophosphate, 1 mM sodium fluoride, 0.1 mM Na3VO4, 0.2 mM PMSF, 5 μg/mL aprotinin, 5 μg/mL leupeptin, 2.5 mM sodium pyrophosphate, and protein concentrations determined by Bradford assay (Bio-Rad). 30-40 μg of whole-cell lysate was loaded onto SDS-PAGE and transferred onto PVDF membrane (Millipore). Blots were probed with the following antibodies diluted into 1% nonfat milk in 10 mM Tris (pH 7.4) saline containing 0.05% Tween-20 (TBS-T): anti-AKT, anti-phospho-AKT (Thr 308), anti-phospho-AKT (Ser 473), all from Cell signaling; anti-PTEN, STAT6 (sc-981) both from Santa Cruz Biotechnology; anti-hemaglutinin (anti-HA, 12CA5) a gift from Dr Martin Scott. Horseradish peroxidase-conjugated anti-rabbit secondary antibody (NA934) and anti-mouse secondary antibody (NA931) were purchased from Amersham. ECL plus chemiluminescence detection system was used to visualize Western blots (RPN 2132, Amersham). In all experiments, equal protein loading was controlled for by either stripping blots as previously described, and reprobing blots with anti-CDK2 or anti-STAT6 (constant level proteins), or in some cases verified by using nonspecific bands for comparison. All Western blot experiments have been confirmed in multiple experiments using separate sets of cell lysates. Data presented in each figure are representative of several independent experiments with similar results.

Generation of chimeric mice with bone marrow cells infected by pca-AKT and pMIGR1 control retroviruses

cDNA encoding a constitutively active form of AKT with a C-terminal HA tag (myr-AKT-HA) was inserted into the MIGR1 plasmid. The MIGR1 vector contains an MSV promoter and a gene encoding GFP. The target gene and GFP tag are separated by an internal ribosomal site (IRES) sequence, thus allowing each protein to be expressed independently (non-fused). 10 mg of each plasmid was used for each transfection. 293T cells were co-transfected with pHIT123 and pCGP using calcium phosphate method (293T cells were seeded the previous day to give a maximum of 70% confluence per plate on the day of transfection). At 48 hours post-transfection, the supernatant was harvested and assayed for viral titer by infection into NIH3T3 cells. The retroviral supernatant was stored at −80° C. until use. Bone marrow of female C57BL/6 mice (8-10 weeks old, purchased from Jackson Labs and kept in specific pathogen-free conditions) were ablated with 5-fluorouracil (5-FU, 250 mg/kg weight) as previously reported. Bone marrow cells (BMCs) were isolated from tibia and femur and suspended in DMEM with 5% heat-inactivated fetal calf serum (FCS). Red blood cells were depleted using ACK lysis buffer as described previously. BMCs were then cultured for one day in 6-well plates with a cocktail of the following cytokines: IL-3 (6 ng/ml), IL-6 (10 ng/ml) and stem cell factor (SCF, 100 ng/ml). Thawed retroviral supernatant was added to BMCs, and cultured for an additional 4-6 days. Cells were collected and injected into lethally irradiated mice (850 rad) as described previously. Immature B cells were harvested at 14 days post-transplant.

### B. Results

BCR-Induced Mitochondrial Depolarization is Independent of Caspases, E2F1, p53 and JNK2 in Immature B Cells

Immature B cells utilized in this study were enriched and purified following the procedure described by Monroe and colleagues (King et al., Immunol Rev 176:86). Specifically, transitional immature B cells were isolated from mouse spleen on day 14 following sublethal irradiation and auto-reconstitution. Cells harvested after 14 days post-irradiation largely consist of immature B cell populations, and can be verified for immature phenotype using cell surface markers against IgM and IgD or CD24 and CD21 expression. Using this method, cells that were 80-90% pure for immature B cells (IgMhi and CD24hi) were obtained.

BCR-induced mitochondrial depolarization is an early critical event that is responsible for initiating late apoptotic program, including DNA fragmentation. To identify molecules that initiate BCR-induced apoptosis, a sensitive and reliable JC-1 staining method was used to monitor the collapse of mitochondrial membrane potential (Gottlieb et al., Cell Death Differ 10:709). Using this assay, it was confirmed that anti-IgM antigen receptor ligation gave rise to rapid mitochondrial depolarization (DYm) in immature B cells. Introduction of Bcl-xL transgene restored mitochondrial membrane potential.

It has been shown that BCR ligation can lead to caspase activation and subsequent cell death in both developing and mature B lymphocytes (Andjelic and Liou. 1998. Eur J Immunol 28:570, Kovesdi et al., 2004. Cell Signal 16:881; Graves et al., Immunol Rev 197:129; Katz et al., 2004. Blood 103:168). Whether caspase activation is related to mitochondrial disruption in primary B cells remains less clear. The effects of caspase inhibition on BCR-induced loss of DYm and DNA fragmentation was examined using a polycaspase inhibitor z-VAD-fmk and a specific inhibitor z-LEHD-fmk for a well-known initiator caspase (caspase 8). Neither inhibitor reversed BCR-induced changes in DY in immature B cells. A specific cathepsin B inhibitor z-FA-fink failed to inhibit anti-IgM induced apoptosis at all. Hence caspases are not required for initiation of mitochondrial-dependent apoptosis.

Previous research in T cell compartment has demonstrated the involvement of several signaling molecules and transcription factors in immune tolerance. In particular, thymocytes derived from JNK2, p53, and E2F1 knockout mice were resistant to antigen receptor mediated apoptosis (Villunger et al., 2003. Science 302:1036; Mihara et al., 2003. Mol Cell 11:577; Lissy et al., 2000. Nature 407:642; Field et al., 1996. Cell 85:549; Zhu et al., Cell Growth Differ 10:829; Sabapathy et al., 2001 J Exp Med 193:317). Furthermore, these molecules have been implicated in activation of the mitochondrial death pathway directly or indirectly. Since there is a strong similarity between TCR and BCR signaling, it was contemplated that some or all of these molecular targets play a role in the initiation of BCR-induced mitochondrial depolarization in immature B cells. To test this, E2FI−/−, p53−/− or JNK2−/− immature B cells were analyzed. JNK2 deletion failed to rescue anti-IgM induced changes in DYm in immature B cells, indicating that inactivation of JNK2 was unable to override the apoptotic signals from the antigen receptor. Similarly, loss of p53 or E2F1 did not block mitochondrial depolarization. Hence despite the essential role in TCR-induced cell death, the absence of these signals is dispensable for both early and late stage BCR-induced cell death events in immature B cells.

### PTEN Inactivation Blocks BCR-Induced Loss of Mitochondrial Integrity and Overrides Cell Cycle Arrest in Immature B Cells

PTEN protein is a phosphatase that counteracts PI3K activity by catalyzing dephosphorylation of phosphatidylinositol-3,4,5-trisphosphate PI(3,4,5)P3 (or PIP3) into phosphatidylinositol-4,5-diphosphate P1(4,5)P2 (or PIP2). PIP3 is an important second messenger that activates downstream effectors including AKT kinase, a potent growth stimulator with anti-apoptotic effects. Previous findings suggest that deletion Pten in T cell compartment leads to loss of central and peripheral immune tolerance. Consequently, Pten-null mice develop autoimmune disease (Di Cristofano et al., 1999. Science 285:2122; Suzuki et al., 2001. Immunity 14:523; Anzelon et al., 2003. Nat Immunol 4:287; Suzuki et al., 2003. J Exp Med 197:657). However, it remains unclear whether PTEN is required for BCR-induced cell death, or if it participates in the initiation of mitochondrial-dependent apoptosis.

Because whole organism deletion of PTEN−/− is lethal, B cell-specifically deleted CD19cre/+PTENflox/flox mice were utilized (bPTEN, generated from breeding PTENflox/+ mice with CD19Cre/+ transgenic mice). Following the sublethal-irradiation and auto-reconstitution procedure, 80-90% pure immature CD19cre/+PTENflox/flox B cells were obtained. Anti-IgM treatment of wild type (CD19cre/+PTEN+/+) immature B cells induces accelerated loss of DYm compared with unstimulated mature and immature B cells, indicating suggesting that antigen receptor ligation transmits a stronger death signal than when cells are cultured in media alone (death by neglect). Loss of PTEN activity in immature B cells appears to negate BCR-induced death signaling and effectively hinders mitochondrial membrane depolarization. Propidium iodide staining further confirmed that deletion of bPTEN not only blocked DNA fragmentation, but also significantly restored BCR-induced cell cycle progression in immature B cells.

It was further observed that the average number of reconstituted immature B cells in bPTENflox/flox mice is markedly greater than in wild type control mice. The results are consistent with previous findings showing that the absolute number of transitional immature B cells, B1, and marginal zone B cells in bPTENflox/flox mice increases by 2-3 fold over wild type (Suzuki et al., supra). In vivo expansion of IgMhi CD24hi cells in bPTENflox/flox mice is thus a consequence of disrupted negative selection processes in the absence of PTEN.

Hence while PTEN is critical for antigen receptor mediated negative selection in immature B cells, E2FI, p53, JNK2, and caspases do not play an essential role, or may engage in molecularly redundant mechanisms that compensate for their loss.

### Elevation of PTEN Activity Silences PI3K/AKT Survival Pathway in Immature B Cells

The potent anti-apoptotic effect of PTEN deletion in BCR-stimulated immature B cells led to an investigation of whether PTEN activity is differentially regulated in mature versus immature B cells upon antigen receptor crosslinking. Studies suggest that in its natural state, PTEN is constitutively active and regulated by several mechanisms, including expression level (Stambolic et al., 2001. Mol Cell 8:317). Using immunoblot assays, it was observed that anti-IgM stimulation resulted in significantly higher PTEN protein levels in immature B cells, which persisted for up to 480 minutes post-stimulation, whereas mature B cells showed little change in PTEN levels. BCR ligation in immature B cells potently and persistently induced the production of a lower molecular weight PTEN isoform that emerged only transiently in mature B cells. While the identity of this protein is unknown, its appearance correlates with increased PTEN activity in immature B cells (see below).

Because PIP3 is the primary endogenous substrate for PTEN, it was next examined whether BCR-mediated PIP3 production was suppressed in immature B cells, due to high Pten activity. Using a PIP3-specific antibody, it was observed that BCR stimulation of mature B cells led to an early (5 min) and late (120 min) wave of intracellular PIP3 accumulation. Decreasing PIP3 levels were observed after 120 min, correlating with the transient expression of the lower molecular weight PTEN isoform. In contrast, antigen receptor stimulation of immature B cells resulted in virtually no detectable increase in intracellular PIP3 levels. Since PIP3 level is determined by counteracting reactions mediated by PTEN and PI3K (i.e. PTEN catalyzes PIP3 to PIP2, PI3K catalyzes the production of PIP3), the low PIP3 level in immature B cells could be due to high PTEN activity or low PI3K activity or both. To discern the possibilities, PI3K activity was measured in these cells. Using immunoprecipitation and TLC-based in vitro kinase assay, it was found that BCR-dependent PI3K activation was reduced in primary immature B cells. In contrast, BCR stimulation of mature B cells led to early and sustained PI3K activation.

PIP3 is an important second messenger that is required for the plasma membrane translocation and activation of many downstream signaling molecules, including Akt, PDK, and PLC-g. In particular, Akt has been considered one of the most critical molecules in the PI3K pathway that mediates growth stimulation and anti-apoptotic functions in a variety of cells. Therefore, low levels of PIP3 may affect proper activation and phosphorylation of AKT protein in immature B cells. Using phospho-specific antibodies, it was observed that BCR stimulation promotes phosphorylation of the downstream anti-apoptotic PIP3-dependent AKT signaling molecule in mature B cells. However, AKT phosphorylation and activation was undetectable in immature B cells.

Since it is unclear whether increased PTEN activity or decreased PI3K activity plays a dominant role in controlling PIP3 level and AKT activation, AKT phosphorylation in mature and immature B cells derived from PTEN knockout or wild type mice was examined. PTEN-deletion significantly enhanced AKT phosphorylation in immature B cells, despite weak PI3K activity in the cells. These results indicate that increased PTEN activity is primarily responsible for BCR-induced AKT inhibition in immature B cells. In summary, higher PTEN expression, lower PI3K activity, and inhibition of PIP3 production was observed in immature B cells. The data suggest that BCR signaling selectively modulates PIP3 levels via control of PI3K activity and expression of PTEN protein depending upon the stage of B cell development (i.e. mature versus immature). As a consequence, the increased PTEN activity uncouples the PI3K/AKT survival pathway and abrogate AKT-mediated survival pathway during BCR signaling in immature B cells to promote cell death.

Restoration of AKT Activity Prevents Immature B Cells from BCR-Induced Cell Death

Since PIP3 activates proteins other than AKT, including the TEC tyrosine kinase family and AGC family serine/threonine kinases such as BTK and PDK1, BCR-induced cell death may involve inactivation of these pathways independently of AKT inhibition. Reports suggest that PTEN-associated mitochondrial depolarization in neuronal cells does not affect AKT activity. These findings led to an investigation of whether restoration of AKT activity alone is sufficient to block BCR-induced apoptosis. A constitutively active form of AKT (myr-AKT-HA) or control vector was transfected into immature B cells by retroviral gene transduction and bone marrow transplantation. BCR engagement led to a significant reduction in the percentage of viable immature B cells in the MIGR1 control group from 12 hours (68%) to 48 hours (33%), a decrease of 35%. In contrast, BCR stimulation of immature B cells infected with constitutively active AKT resulted in only a modest decrease of viable immature B cells from 12 hours (76%) to 48 hours (69%).

These data are consistent with findings in PTEN-deleted immature B cells, and indicate that restoration of AKT activity by forced expression of constitutively active AKT inhibits BCR-induced immature B cell death. The results demonstrate that constitutive AKT activation can fully block spontaneous apoptosis, indicating that PTEN plays a selective role in initiating BCR-mediated cell death but not in death-by-neglect signaling.

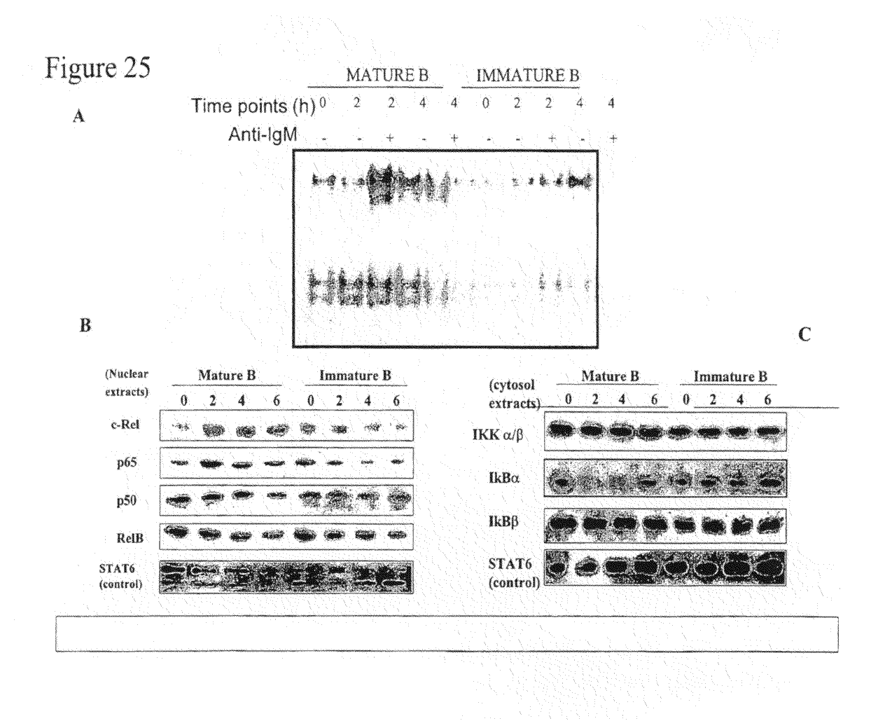

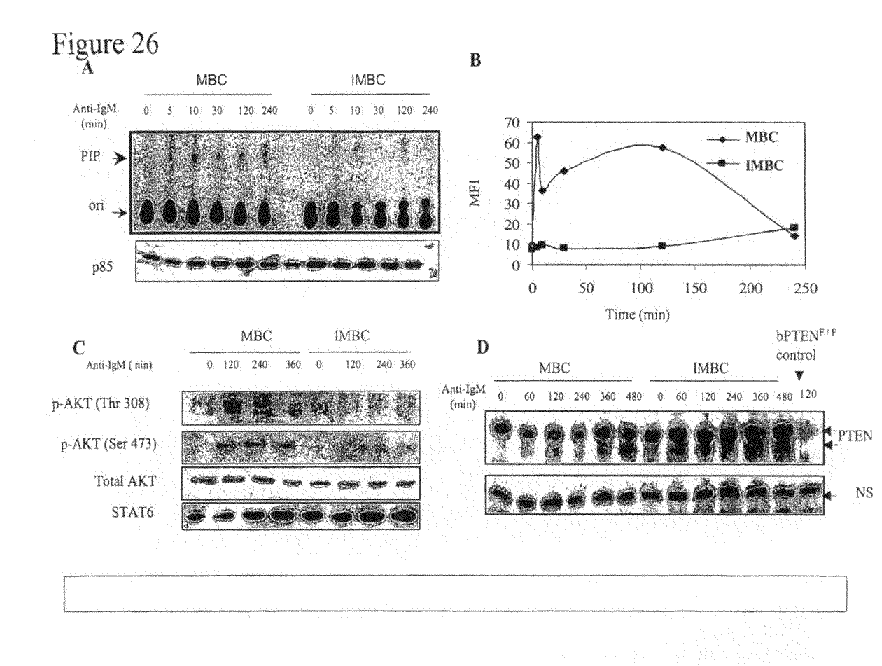

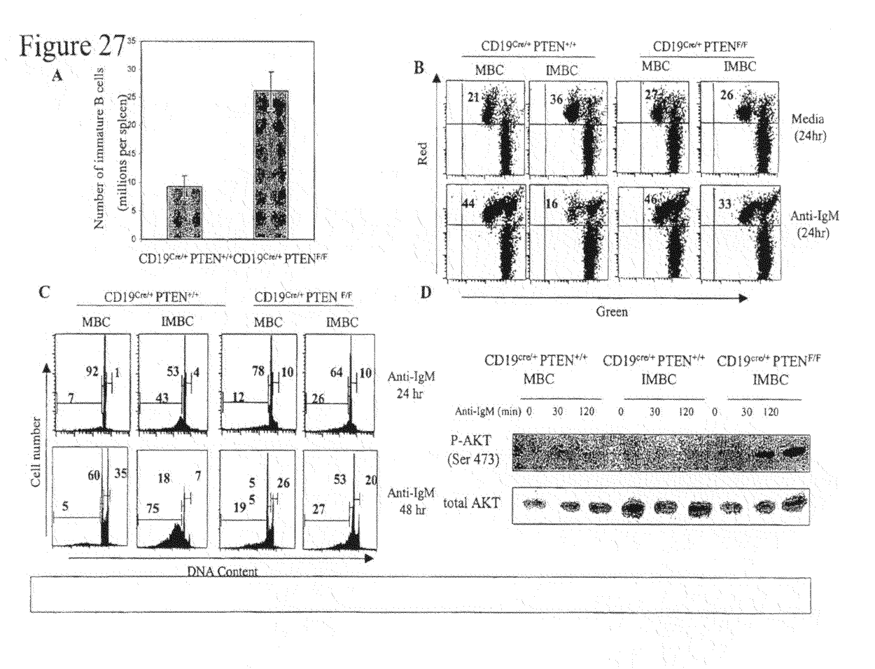

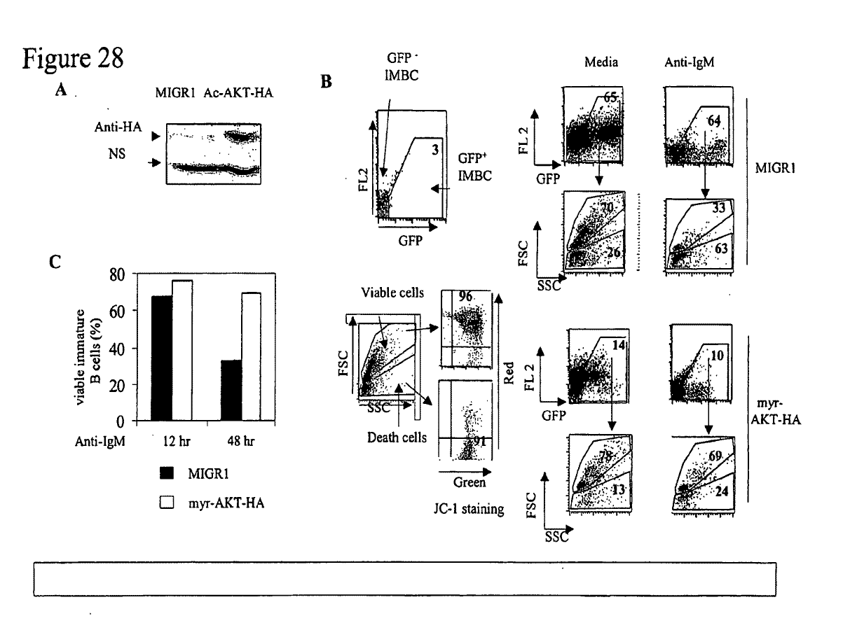

Immune tolerance to a specific antigen (or tissue) can be achieved through three major mechanisms: deletion, anergy, and T-regulatory cells. c-Rel inhibition is associated with these three immune tolerance mechanisms. Anergic T cells and anergic B cells, which are unresponsive to TCR/BCR stimulation, have specific blocks in the c-Rel and NF-kB pathway (FIG. 25). Studies on immature B cells, which undergo deletion, have a specific block in their c-Rel/NF-kB and PI3K activation pathway. Conversely, activation of the PI3K-Rel/NF-kB pathway in tolerant cells can lead to immune tolerance breakdown and the onset of autoimmune diseases (FIGS. 26-28). In addition, recent studies have suggested that suppression of the NF-kB/Rel and NFAT by the FoxP3 is the underlying mechanism by which T-regulatory cells suppress effector T cell function. Finally, there is ample evidence to support that blocking NF-kB/Rel activity in dendritic cells can prevent maturation of dendritic cells and that such immature dendritic cells induce T cell tolerance or T-regulatory cell differentiation. Together, these studies indicate that the c-Rel/PI3K pathway is the signaling integration point for determining immune tolerance vs autoimmunity. More specifically, sustained activation of this pathway leads to autoimmune diseases, whereas suppression of this pathway induces immune tolerance.

### Example 9

This Example describes the use of expression microarrays to identify c-Rel targets.

### Materials and Methods

Mice, purification of murine splenic B cells. c-rel+/+ and c-rel−/− mice (all on a C57BL/6 background) were generated as described previously (Owyang, 2001 supra). Experiments were conducted using 8-12 week old mice maintained under specific pathogen-free condition. Purified B cells (>95% B220+) were enriched by complement-mediated lysis as described previously, and cultured in complete media containing RPMI 1640, 10% FCS (Cellgro), 1% penicillin, 1% streptomycin (both Life Sciences BRL), and 50 mM b-mercaptoethanol (Sigma).

Cell culture. Wild type and c-rel−/− B cells were stimulated for 4 hours with 10 mg/mL agonistic antibodies against the BCR (F(ab)′2 goat anti-mouse m-chain from Jackson Immunotech) or anti-CD40 (hybridoma 1C10) using complete media as a control. To limit the effects of c-Rel transcription on direct targets only, 10 mg/mL cycloheximide was used to block protein synthesis and prevent secondary gene transcription.

[³H] Thymidine incorporation. To verify c-rel associated proliferation defects, 1×10⁵ B cells were cultured in 96-well U-bottom plates with 10 mg/mL anti-BCR or 10 mg/mL anti-CD40 for 42 hrs, then pulsed for additional 6 hrs with 0.5 mCi [³H] thymidine. Cells were then harvested and measured for the amount of DNA-incorporated [3H] by scintillation counter as described previously.
Microarray analysis. RNA samples were prepared as described previously (Owyang, 2001, supra) and biotinylated strepavidin-labeled oligonucleotide probes generated according to manufacturer suggested protocols (Affymetrix). Samples were then assayed for gene expression profiles using Affymetrix U74A microarray chips, and statistical analysis (ANOVA) performed using Genespring v.6.0 software. Approximately 6000 known genes and 6000 expressed-sequence tag (EST) sequences were evaluated on each microarray chip. To determine differential gene expression between samples, a minimum 2-fold difference was set as the threshold criteria and the data filtered to obtain p values of p≦0.02. Experiments were performed in duplicate or triplicate to ensure high reproducibility of results. Data from each experiment was normalized to average media control values. Genes were then clustered by expression profile and screened for known or unknown immunological relevance.
Immunoblotting. Cells were lysed in 50 mM Hepes (pH 7.5), 150 mM NaCl, 1 mM EDTA, 2.5 mM EGTA, 1 mM dithiothreitol, 0.1% Tween-20, 10% Glycerol, 10 mM b-glycerophosphate, 1 mM sodium fluoride, 0.1 mM Na3VO4, 0.2 mM PMSF, 5 mg/mL aprotinin, 5 mg/mL leupeptin, 2.5 mM sodium pyrophosphate, and protein concentrations determined by Bradford assay (Bio-Rad). 30-40 micrograms of whole cell lysate were loaded onto SDS-polyacrylamide gels and transferred to PVDF membrane (Millipore). Blots were probed with the following antibodies diluted into 1% nonfat milk in tris buffered saline solution containing 0.05% tween-20 (TBS-T): EBI3 and Amphiregulin from R&D Systems. Horseradish peroxidase conjugated anti-rabbit secondary antibody (NA934) and anti-mouse secondary antibody (NA931) were purchased from Amersham. ECL plus chemiluminescence detection system was used to visualize western blots (RPN 2132) from Amersham. In all experiments, equal protein loading was controlled for by either stripping blots as previously described, and reprobing blots with anti-CDK2 or anti-STAT6 which are constitutively expressed in B cells, or verified by using non-specific bands for comparison. Western blot experiments have been confirmed in multiple experiments using separate sets of cell lysates.
RT-PCR. Splenic B cells were cultured in 10 mg/mL anti-BCR or 10 mg/mL anti-CD40 for a duration of 4 hrs. Total RNA preparation and reverse transcription were carried out as described. Genes were amplified by PCR using the following mouse specific primers against ebi3: forward 5′-GTGCAATGCCATGCTTCTC-3′, reverse 5′-TGCCACCCT CAAGTAGACG-3′ with an expected size of 648 bp. PCR products were separated on 2% agarose in tris-acetate-EDTA buffer and visualized by ethidium bromide staining.

### Results

Statistical analysis of anti-BCR and anti-CD40 stimulated B cells. Wild type and c-rel−/− B cells were stimulated for 4 hours with agonistic antibodies against the BCR (anti-BCR) or CD40 (anti-CD40) using media as a control. To limit the effects of c-Rel on direct transcriptional targets only, cycloheximide was used to block protein synthesis and prevent secondary gene transcription. Experiments were performed in triplicate to ensure high reproducibility of results. Samples were then assayed for gene expression profiles using Affymetrix U74A microarray chips, and statistical analysis (ANOVA) performed using Genespring v.6.0 software. Approximately 6000 known genes and 6000 expressed-sequence tag (EST) sequences were evaluated on each microarray chip. To determine differential gene expression between samples, a minimum 2-fold difference was set as the threshold criteria and the data filtered to obtain p values of p≦0.02. Data from each experiment was normalized to average media control values. Genes were then clustered by expression profile and screened for known or unknown immunological relevance.
Comparing wild type and c-rel−/− responses, it was found that the number of genes exhibiting a statistically significant difference included 134 genes for anti-BCR treatment (p≦0.01) and 89 genes for anti-CD40 treatment (p≦0.01). Within these two populations 66 genes for anti-BCR treatment and 48 genes for anti-CD40 treatment expressed at least 2-fold difference between wild type and c-rel−/− B cells. Only 5 genes were common to both anti-BCR and anti-CD40 treatment including growth factors, anti-apoptotic molecules, and cytokines. These results, combined with the magnitude of the deficiencies in c-Rel dependent gene activation (see below), indicate that c-rel deficiency results in loss of unique gene expression for each stimuli.

Gene expression induced by anti-BCR and anti-CD40 treatment was also evaluated in wild type B cells alone to confirm the effect of these two signaling molecules on normal B cell activation. Comparing normal responses to media controls, it was found that 271 genes are induced by anti-BCR treatment (p≦0.005), while 671 genes are induced by anti-CD40 treatment (p≦0.02) (Table 3). Within these two populations, 212 genes exhibit ≧2-fold induction for anti-BCR stimulation and 391 genes show ≧2-fold induction for anti-CD40 treatment. It was determined that roughly 52 genes were in common for these two conditions, however this value under-represents the true number of common responses since the minimum 2-fold threshold difference eliminates many commonly induced genes. Nevertheless, the data suggest that roughly a quarter of the genes induced by anti-BCR stimulation overlap with genes induced by anti-CD40 treatment including transcription factors, growth factors, survival factors, surface receptors, intracellular signaling molecules, and various genes of immunomodulatory function (Table 3). It was additionally found that a number of these common genes were repressed by anti-BCR and anti-CD40 stimulation including transcriptional repressors and adaptor molecules.

TABLE 3

| | | >2 fold | total no. | |
|---|---|---|---|---|
| | | change | genes | p value |
| WT | BCR | 212 | 271 | 0.005 |
| | CD40 | 391 | 671 | 0.02 |
| | common genes | 52 | | |
| WT vs KO | BCR | 66 | 134 | 0.01 |
| | CD40 | 48 | 89 | 0.01 |
| | common genes | 5 | | |

### BCR and CD40-Activated C-Rel−/− B Cells Fail to Express the Bcl-xL Survival Gene

Previously it was reported that the anti-apoptotic gene, bcl-xL, is a direct target gene of c-Rel during BCR and CD40 stimulation of mouse B lymphocytes. Hence c-rel deficient mice expressing the bcl-xL transgene are corrected for survival defects against a variety of cellular insults including BCR-induced apoptosis. DNA microarray analysis confirms this dramatic under-expression of bcl-xL in anti-BCR and anti-CD40 treated c-rel−/− B cells.

Comparing wild type and c-rel−/− B cells, a 13-fold loss of bcl-xL induction in both anti-BCR and anti-CD40 treated c-rel deficient B cells was observed. The findings therefore validate this technique to measure the activation of immediate early target genes.

Growth factor, cytokine, and chemokine expression in c-rel−/− B cells. Although it is speculated that c-Rel controls the expression of several important growth factors and cytokines in lymphocytes, only a few genes have been identified to date. Initial findings have shown that c-rel−/− T cells, for instance, are impaired for IL-2 gene expression resulting from lack of gene transcription at the kB response element in the IL-2 promoter. As a result, defective T cell proliferation responses are observed, which can be restored upon treatment with exogenous IL-2. In conjunction with this discovery, several other cytokines known to be expressed in B cells have been identified as c-Rel target genes including IL-6, IL-10, and LT-b.

IL-10 is a pleiotropic T helper cell 2 (TH2) cytokine whose most apparent effects involve the suppression of inflammatory and adaptive immune responses. Studies show that IL-10 affects B cell survival, and the observed loss of IL-10 gene expression in anti-BCR and anti-CD40 treated c-rel−/− cells by microarray analysis (both 3-fold) indicates the importance of this cytokine in B cell activation. Overexpression of IL-10 has been detected in numerous B cell malignancies including murine B cell lymphomas; chronic lymphocytic leukemia; diffuse large B cell lymphoma; indolent B cell lymphoma; EBV-positive Burkitt's lymphoma, cutaneous B cell lymphoma, primary effusion lymphoma, and classical Hodgkin's lymphoma. The data described herein validate the overexpression of IL-10 in several B cell cancers through dysregulation of c-Rel activity.

Differences in IL-6 and LT-b gene expression were not observed in the absence of c-Rel at 4 hours by microarray analysis, but since LT-b expression is activated primarily in germinal center B cells and not in naïve lymphocytes, it was contemplated that loss of LT-b expression may actually occur at later time points or alternatively LT-b gene induction may be induced by other complementary transcription factors. Similarly, IL-6 production primarily occurs during late stage B cell maturation into plasma cells, and hence imperceptible differences in IL-6 gene expression by microarray analysis may again reflect differences in the B cell maturation stage or the redundancy of other transcription factors. Impaired IFNa11 gene expression (4-fold) was found in the absence of c-rel under anti-CD40 stimulating conditions, indicating that c-Rel is involved in the production of paracrine TH1 cytokines.

Decreased expression of ebi3, a subunit of IL-27, was also detected in c-rel deficient B cells by approximately 4-fold in anti-BCR treated cells and 8-fold in anti-CD40 treated cells. RT-PCR experiments independently confirm this decrease at the RNA level while further analysis by western blot verifies that EBI3 protein expression is reduced in the absence of c-rel. EBI3 is related to the p40 subunit of IL-12, and has been shown to heterodimerize with p28, a homologue of the p35 subunit of IL-12, to form IL-27. Although originally identified in EBV-transformed B cells, EBI3 is expressed primarily in monocytes and dendritic cells and has been detected in Reed-Sternberg cells and Hodgkin's lymphoma cells as well. Reports show that EBI3 may also play a role in inflammatory bowel disease. Corroborating with the studies described herein, it was shown by others that deletion of ebi3 in mice impairs TH1 responses similar to IL-12 deficient mice, and corresponds with loss of IFN-g production. However loss of ebi3 may also affect TH2 responses by impairing IL-4 production and interfering with natural killer cell (NK) responses. The data described herein indicate that induction of ebi3 by c-Rel represents a novel mechanism of gene activation, and involves a means by which c-rel associated lymphomagenesis and autoimmunity evolves.

Another newly identified target gene detected in the assays was the amphiregulin growth factor. amphiregulin is significantly underexpressed by 15-fold in both anti-BCR and anti-CD40 treated B cells, although the difference appears more dramatic in anti-CD40 treated cells. Western blot analysis reveals that the Amphiregulin protein is decreased in c-rel−/− B cells compared to wild type cells. Amphiregulin is an EGF-related protein which has been implicated in numerous cancers including breast carcinoma, prostate cancer, colon cancer, kidney cancer, bladder cancer, squamous cell carcinoma, lung cancer, pancreatic cancer, ovarian cancer, and keratinocytic tumors. Although expression of Amphiregulin in lymphocytes has not been reported, the analysis described herein shows that this gene is dramatically reduced in anti-CD40 stimulated c-rel deficient B cells. Its induction in B cells by both BCR and CD40 signaling indicates that this molecule functions in lymphoid activation and humoral immunity. Dysregulation of amphiregulin has not been identified in any lymphoid malignancies, presumably due to its predominant gene expression patterns in epithelial tissue rather than hematopoietic cells. Production of Amphiregulin by B cells may therefore contribute to the malignancy of epithelial-derived cancers instead of lymphoid-derived tumors.

Genes that are induced by BCR signaling in the absence of c-rel include interferon alpha receptor (IFNaR) (3-fold), egfbp1/kik22 (8-fold), and IL-12Rb1 (9-fold). These results indicate that BCR stimulation normally suppresses the activation of these genes in a c-Rel dependent manner. Since IFNa and IL-12 act primarily on CD8+ TH1 cells, and EGFBP is an epidermal growth factor binding protein, the suppression of these genes reflects the inability of B cells to respond to these factors under normal conditions. Meanwhile, anti-CD40 treatment results in the induction of serum amyloid A protein 2 (saa2) (3.4-fold), guanine nucleotide binding protein 1 (gbp1) (13-fold) in the absence of c-rel. These data indicate that c-Rel possesses repressor activity in addition to its transcriptional activation functions.

Signaling molecule expression in c-rel−/− B cells. Because c-Rel activates multiple signal transduction pathways, the loss or induction of signaling molecules in c-rel−/− B cells was next evaluated. Relatively few signaling molecules are affected by the loss of c-rel. A reduction in the proto-oncogenic kinase pim1 (2-fold) upon anti-BCR treatment was detected. Pim1 is a serine-threonine kinase encoded by the pim1 proto-oncogene that is activated by both BCR and CD40 signaling. It is expressed primarily in hematopoietic lineages, and has reported functions in the growth, survival, and differentiation of B cells. Mouse tumor models demonstrate that overexpression of pim1 in the lymphoid compartment leads to lymphomagenesis, and dysregulation of pim1 appears to be involved bovine leukemia as well. The observed loss of pim1 gene expression in c-rel−/− B cells indicates the importance of this factor in Rel dependent B cell activation.

Another gene, phospholipase A gamma 2 (plag2), has been shown to be induced by BCR signaling which mediates B cell proliferation through a calcium-independent mechanism. plag2 levels are reduced by 5-fold in BCR-activated c-rel−/− B cells, and correlate with loss of plag21br (6-fold).

Loss of map21k/mek1/erk1 expression (2.5-fold) was observed in CD40-treated c-rel−/− B cells, while induction of the p38 kinase mapk13 (5-fold) was seen upon anti-BCR treatment. These results indicate that members of the MAP kinase signaling pathway are also acutely regulated by c-Rel during B cell activation and costimulation.

CD40 stimulation resulted in 3-fold higher induction of ikk-i in wild type B cells than in c-rel−/− B cells. Other NFkB signaling pathway molecules do not appear to be affected in the absence of c-rel. Analysis of wild type CD40 shows induction of ikk-i, ikk-b, ikb-b, and ikb-e.

Microarray analysis does not reveal loss of cell cycle gene expression in c-rel mutants. The results are consistent with the finding that c-Rel dependent cell cycle defects occur only after 12 hours stimulation, and furthermore, are associated with secondary gene expression events which are not detectable in the experiments due to the inclusion of cycloheximide in the cultures.

Non-immune response gene expression in c-rel−/− B cells. The advantage of microarray analysis is that it provides analysis of a wide range of genes that have not been previously associated with c-Rel activation. The identification of new c-Rel target genes is therefore made possible using this technique. Several “non-immune” response factors were found to exhibit c-Rel dependence. These include the estrogen receptor (ER) (2.1-fold), G-protein coupled receptor girk1 (5.4-fold), G-protein gamma subunit gng4 (3.8-fod), NKp46-related receptor mar1 (11-fold), nicotinic acetylcholine receptor chrnd (5.4-fold), amelogenic matrix protein ameloblastin (ambn) (5.2-fold), lysbsomal transport GTPase rab34 (9.9-fold), synaptic vesicle protein syt9 (5.8-fold), metabolic enzyme biphosphoglycerate mutase (bpgm) (2.2-fold), cathepsin H (ctsH) (2.1-fold), and the seminal vesicle protein F (svsS) (2-fold).

Deletion of c-rel, leads to significant upregulation of beta-adrenergic receptor adra1b (6.6-fold), cannabinoid receptor cb2 (2.2-fold), Ras-like GTP-binding protein r-rad (2-fold), multidrug resistance protein nrp6 (3.2-fold), adhesion molecule madcam1 (2.7-fold), myosin heavy chain myh3 (3.7-fold), sarcomeric myosin-binding protein mybpc3 (3.9-fold), synaptotagmin syt2 (2.1-fold), cosinophil secondary granule ribonuclease m-ear1 (4.1-fold), and transcriptional regulator hox8 (2.4-fold), signifying that suppression of these genes normally occurs in response to BCR and CD40 stimulation.

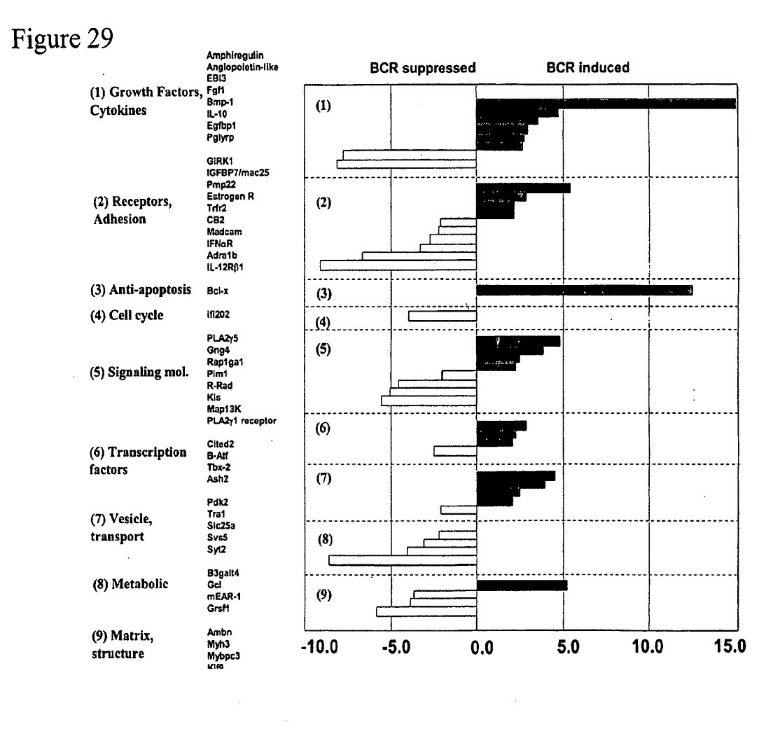

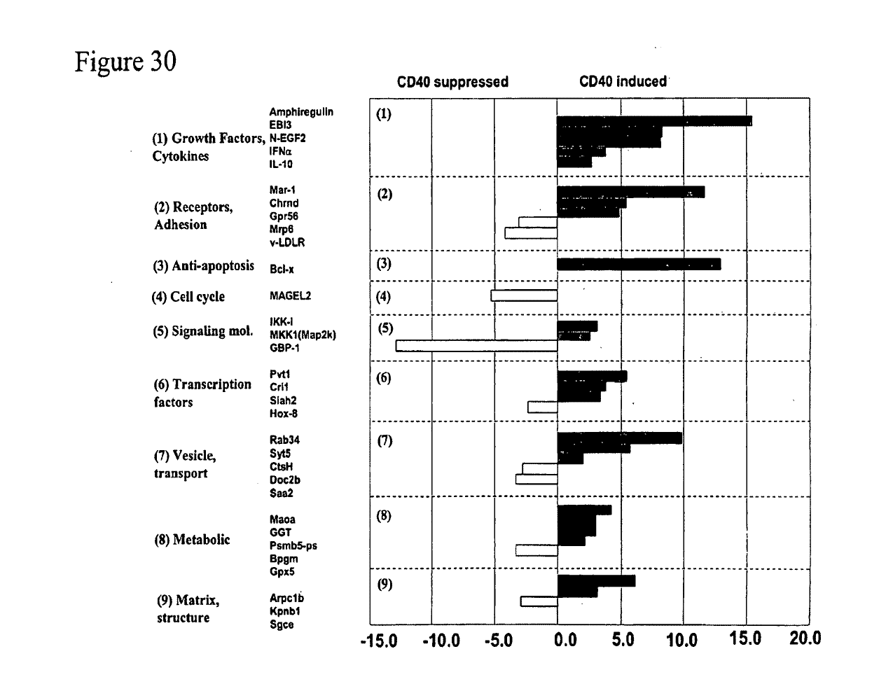

A transcription factor, such as c-Rel, exerts its function through the regulation of target genes. Therefore, identifying c-Rel target genes is useful to provide further targets in inflammation, autoimmune disease, and tumorigenesis. DNA microarray technology is used to identify c-Rel target genes. The c-Rel knockout mice provide a unique advantage to specifically identify c-Rel target genes. By comparing the expression of genes between c-Rel wild type and c-Rel knockout cells, it was shown that it is possible to identify many novel genes that are regulated by c-Rel. FIG. 29 and FIG. 30 demonstrate that c-Rel is capable of regulating distinct and diverse sets of genes, depending on the context of stimuli and cell types. Furthermore, c-Rel target genes include novel growth factors, soluble factors, signaling molecules, transcription factors, cell cycle, and anti-apoptotic proteins, which are not described previously in the literature. These target genes provide functions that fuel the inflammation and cancer, by serving as autocrine factors, paracrine factors, survival proteins, differentiation, or metastasis.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of inhibiting c-Rel activity, comprising contacting a eukaryotic cell expressing a c-Rel gene with a c-Rel activity inhibitor.

2-26. (canceled)

27. A method of inhibiting the expression of a c-Rel target gene, comprising contacting a eukaryotic cell expressing a c-Rel gene with a c-Rel activity inhibitor.

28-30. (canceled)

31. A method, comprising contacting a eukaryotic cell exhibiting abnormal signaling effects, wherein said abnormal signaling effects result in altered c-Rel activity with a c-Rel activity inhibitor under conditions such that said abnormal signaling effect is diminished.

32. (canceled)

33. A method for modifying extracellular signaling influences on a eukaryotic cell, wherein said extracellular signaling induces c-Rel activity, comprising contacting said cell with a c-Rel activity inhibitor under conditions such that said signaling effect is decreased.

34. A method of treating a disease caused by excessive c-Rel activity, comprising administering a c-Rel activity inhibitor to a subject exhibiting symptoms of said disease.

35-40. (canceled)

41. A method of treating an autoimmune disease or transplant rejection by immune therapy, wherein said immune therapy comprises administrating a c-Rel inhibitor to a subject exhibiting symptoms of autoimmune disease or transplant rejection.

42. A method of treating a disease caused by aberrant expression of a c-Rel target gene, comprising administering a c-Rel activity inhibitor to a subject exhibiting symptoms of said disease.

43. A kit comprising a c-Rel activity inhibitor in a pharmaceutically acceptable carrier.

44-53. (canceled)

54. A composition comprising a small molecule, wherein said small molecule has a structure selected from the group consisting of: 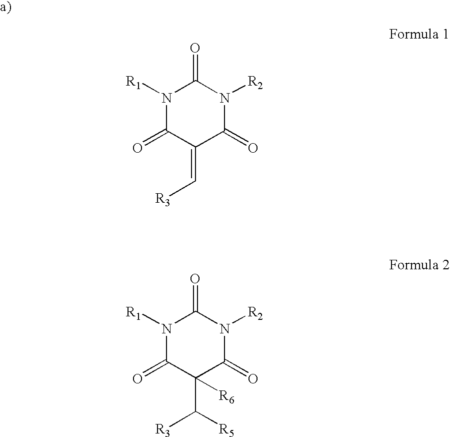
wherein R₁, R₂, R₅ and R₆ are independently selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl; R₃ is selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, CN, NO₂, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁; wherein R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl; 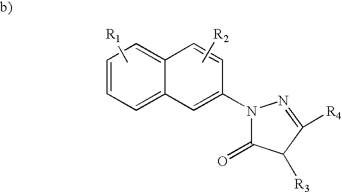
wherein R₁ and R₂ are independently selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, OH, OR₁₁, SH, SR₁₁, NO₂, CN, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁; R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl; R₁₁ and R₁₂ can be connected to form a cycle which can be heterocyclic aromatic or non-aromatic, substituted heterocyclic aromatic, cycloakyl, substituted cycloalkyl; R₃ and R₄ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl; R₃ and R₄ can be connected to form a cycle which can be heterocyclic aromatic or non-aromatic, substituted heterocyclic aromatic, cycloakyl, substituted cycloalkyl; and 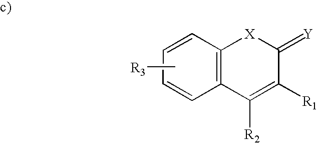
wherein X and Y are independently selected from NH, NR₄, O and S; R₁, R₂ and R₄ are independently selected from hydrogen, aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocylic aromaric or non-aromatic, substituted heterocyclic aromatic or non-aromatic, cycloalkyl, substituted cycloalkyl; R₁ and R₂ can be connected to form a cycle which can be heterocyclic, substituted heterocyclic, cycloakyl, substituted cycloalkyl; R₃ is selected from hydrogen, aryl, substituted aryl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl, halogen, COR₁₁, OH, OR₁₁, SH, SR₁₁, NO₂, CN, SO₂R₁₁, NR₁₁R₁₂, NR₁₂(CO)OR₁₁, NH(CO)NR₁₁R₁₂, NR₁₂(CO)R₁₁, O(CO)R₁₁, O(CO)OR₁₁, O(CS)R₁₁, NR₁₂(CS)R₁₁, NH(CS)NR₁₁R₁₂, NR₁₂(CS)OR₁₁; R₁₁ and R₁₂ are independently selected from hydrogen, aryl, aralkyl, substituted aralkyl, alkyl, substituted alkyl, alkenyl, substituted alkenyl, alkynyl, substituted alkynyl, halogenated alkyl, halogenated alkenyl, halogenated akynyl, arylalkyl, arylalkenyl, arylalkynyl, heterocyclic aromatic or non-aromatic, substituted heterocyclic aromatic or non-aromatic, cycloalkyl, substituted cycloalkyl; R₁₁ and R₁₂ can be connected to form a cycle which can be heterocyclic aromatic or non-aromatic, substituted-heterocyclic aromatic, cycloakyl, substituted cycloalkyl.

55. (canceled)

56. A composition comprising a small molecule selected from the group consisting of 1H-Pyrazole-1-butanoic acid, 3-(4-bromophenyl)-5-(1,2-dihydro-7-methyl-2-oxo-3-quinolinyl)-4,5-dihydro-g-oxo-(9CI) 1,5-Naphthalenedisulfonic acid, 3-(4,5-dihydro-3-methyl-5-oxo-1H-pyrazol-1-yl)-1,3-Naphthalenedisulfonic acid, 7-(3-methyl-5-oxo-2-pyrazolin-1-yl)-(8CI) butanedioic acid, [5-[(4-hydroxy-3-methoxyphenyl)methylene]-4-oxo-2-thioxo-3-thiazolidinyl], 4-Hydroxy-2-oxo-1,2-dihydroquinoline-3-carboxylic acid N-(4-hydroxyphenyl)amide and 7-(diethylamino)-3-[5-(2,5-dimethoxyanilino)-1,3,4-thiadiazol-2-yl]-2H-chromen-2-one.

57-58. (canceled)

59. A composition comprising an siRNA, wherein said siRNA has the nucleic acid sequence selected from the group consisting of SEQ ID NOs: 6, 10, 13, 16 and 17-26.


SEQUENCE LISTING (37 sequences)

SEQ ID NO: 1 - DNA, 39 bp, Artificial Sequence
  synthetic
gagaggggactttccgattagctttcggaaagtcccctc

SEQ ID NO: 2 - DNA, 19 bp, Artificial Sequence
  synthetic
gtgcaatgccatgcttctc

SEQ ID NO: 3 - DNA, 19 bp, Artificial Sequence
  synthetic
tgccaccctcaagtagacg

SEQ ID NO: 4 - DNA, 29 bp, Artificial Sequence
  synthetic
ggaagatctatccgacgccgccatctcta

SEQ ID NO: 5 - DNA, 43 bp, Artificial Sequence
  synthetic
gtggaattcgttaacgaagaccacaaacaaggcttttctccaa

SEQ ID NO: 6 - DNA, 61 bp, Artificial Sequence
  synthetic
tttggtgtgaagggcgatcagcaggttcaagagacctgctgatcgcccttcacactttttc

SEQ ID NO: 7 - DNA, 61 bp, Artificial Sequence
  synthetic
aattgaaaaagtgtgaagggcgatcagcaggtctcttgaacctgctgatcgcccttcacac

SEQ ID NO: 8 - DNA, 19 bp, Artificial Sequence
  synthetic
aatgtgaagggcgatcagc

SEQ ID NO: 9 - DNA, 19 bp, Artificial Sequence
  synthetic
aatgtgaagggcgatcagc

SEQ ID NO: 10 - RNA, 19 nt, Artificial Sequence
  synthetic
gcugaucgcccuucacauu

SEQ ID NO: 11 - DNA, 23 bp, Artificial Sequence
  synthetic
aatgtgaagggcgatcagcaggt

SEQ ID NO: 12 - DNA, 23 bp, Artificial Sequence
  synthetic
acctgctgatcgcccttcacatt

SEQ ID NO: 13 - RNA, 23 nt, Artificial Sequence
  synthetic
accugcugaucgcccuucacauu

SEQ ID NO: 14 - DNA, 21 bp, Artificial Sequence
  synthetic
gtgtgaagggcgatcagcagg

SEQ ID NO: 15 - RNA, 19 nt, Artificial Sequence
  synthetic
ccgugcuccaaauacugca

SEQ ID NO: 16 - RNA, 19 nt, Artificial Sequence
  synthetic
ugcaguauuuggagcacgg

SEQ ID NO: 17 - DNA, 20 bp, Artificial Sequence
  synthetic
tgtgaagggcgatcagcagg

SEQ ID NO: 18 - DNA, 23 bp, Artificial Sequence
  synthetic
ccgaacatacccttctatccaga

SEQ ID NO: 19 - DNA, 22 bp, Artificial Sequence
  synthetic
gactgcagagacggctactatg

SEQ ID NO: 20 - DNA, 21 bp, Artificial Sequence
  synthetic
ggcaggaatcaatccattcaa

SEQ ID NO: 21 - DNA, 23 bp, Artificial Sequence
  synthetic
gattgtgacctcaatgtggtgag

SEQ ID NO: 22 - DNA, 23 bp, Artificial Sequence
  synthetic
gattgtgacctcaatgtggtgag

SEQ ID NO: 23 - DNA, 22 bp, Artificial Sequence
  synthetic
gctgatgtacaccgtcaagtag

SEQ ID NO: 24 - DNA, 23 bp, Artificial Sequence
  synthetic
gcagaatcctactatccctcacc

SEQ ID NO: 25 - DNA, 21 bp, Artificial Sequence
  synthetic
gagacttgagacagctccatc

SEQ ID NO: 26 - DNA, 22 bp, Artificial Sequence
  synthetic
gatagtcagtattcaggtattg

SEQ ID NO: 27 - DNA, 21 bp, Artificial Sequence
  synthetic
gtgtgaagggcgatcagcagg

SEQ ID NO: 28 - DNA, 20 bp, Artificial Sequence
  synthetic
gcctcatcctcatgatttag

SEQ ID NO: 29 - DNA, 23 bp, Artificial Sequence
  synthetic
gcagaatttggaccagaacgcag

SEQ ID NO: 30 - DNA, 21 bp, Artificial Sequence
  synthetic
ggattagtgcaggaatcaatc

SEQ ID NO: 31 - DNA, 21 bp, Artificial Sequence
  synthetic
gactgcgacctcaatgtggtg

SEQ ID NO: 32 - DNA, 22 bp, Artificial Sequence
  synthetic
gatggtaacttcacaactgctg

SEQ ID NO: 33 - DNA, 20 bp, Artificial Sequence
  synthetic
ggatcttagcccgtgtgaac

SEQ ID NO: 34 - DNA, 20 bp, Artificial Sequence
  synthetic
ggatcttagcccgtgtgaac

SEQ ID NO: 35 - DNA, 22 bp, Artificial Sequence
  synthetic
gctatactggagcctgtgacag

SEQ ID NO: 36 - DNA, 23 bp, Artificial Sequence
  synthetic
gctgaaccttactattcttcatg

SEQ ID NO: 37 - DNA, 20 bp, Artificial Sequence
  synthetic
tctggaatttctttaaaccc